(19) United States
(12) Patent Application Publication
(10) Pub. No.: US 2016/0083441 A1
(43) Pub. Date: Mar. 24, 2016

(54) Development of Protein-Based Biotherapeutics That Penetrate Cell-Membrane and Induce Anti-Cancer Effect - Cell-Permeable Trefoil Factor 1 (CP-TFF1) in Gastrointestinal Track (GIT) Cancer, Polynucleotides Encoding The Same, and Anti-Cancer Compositions Comprising The Same

(71) Applicants: DAEWOONG JO, Brentwood, TN (US); Cellivery Therapeutics, Inc., Seoul (KR)

(72) Inventors: Daewoong JO, Brentwood, TN (US); Young Sil CHOI, Seoul (KR); Kuy Sook LEE, Gyeonggi-do (KR); Hyeon Sik KANG, Gyeonggi-do (KR); Cho Hyun KIM, Incheon (KR)

(21) Appl. No.: 14/863,478

(22) Filed: Sep. 24, 2015

Related U.S. Application Data
(60) Provisional application No. 62/054,406, filed on Sep. 24, 2014.

(51) Int. Cl. C07K 14/47; C07K 14/195; C07K 14/415; C07K 7/08

(52) U.S. Cl. CPC C07K 14/47; C07K 7/08; C07K 14/195; C07K 14/415; A61K 38/00

(57) ABSTRACT

The present study investigated the use of macromolecule intracellular transduction technology (MITT) to deliver biologically active TFF1 protein into gastric cancer cells both in vitro and in vivo. Proteins engineered to enter cancer cells are supposed to suppress cell proliferation and survival, consistent with its role as a tumor suppressor. The invention has developed new hydrophobic CPP-advanced MTDs (aMTDs) for high solubility/yield and cell-/tissue-permeability of the recombinant therapeutic fusion proteins. The TFF1 protein has been fused to aMTD165 and solubilization domains (SDs), and tested their therapeutic applicability as a gastric cancer-specific protein-based anti-cancer agent. Treatment with CP-TFF1 in gastric cancer cells reduced cancer cell viability (60%˜80% in 10 μM treatment), inhibited cell migration (approximately 50%). Furthermore, CP-TFF1 significantly inhibited the tumor growth during the treatment and the effect persisted for at least 3 weeks after the treatment was terminated (90% inhibition at day 42) in a xenografts model which were subcutaneously implanted with tumor block of gastric cancer cells (MKN45). In the present invention, CP-TFF1 recombinant protein showed the potential of novel protein therapies against gastric cancer.

### CROSS-REFERENCE TO RELATED APPLICATIONS

This application claims the benefit of the filing date of U.S. Provisional Application No. 62/054,406, filed on Sep. 24, 2014, in the United States Patent and Trademark Office, the disclosure of which is incorporated herein in its entirety by reference.

### TECHNICAL FIELD

In the present invention, we adopted the use of macromolecule intracellular transduction technology (MITT) to deliver biologically active TFF1 protein into gastric cancer cells, grown both in culture and as tumor xenografts and to investigate the feasibility of using TFF1 as a protein-based therapy for gastric cancer.

### BACKGROUND ART

Gastric cancer remains the fourth most common cancer worldwide and the second leading cause of cancer-related deaths. The most common form of gastric cancer is intestinal-type gastric adenocarcinoma, which progresses through a cascade of gastric carcinogenesis from normal mucosa to chronic superficial gastritis, atrophic gastritis, intestinal metaplasia with low- and high-grade dysplasia (LGD and HGD, respectively), and invasive gastric adenocarcinoma finally.

Trefoil factor 1 (TFF1) is a member of the trefoil factor family peptides that are cysteine-rich proteins and form a characteristic trefoil domain. TFF1 is expressed predominantly in the gastric epithelia and secreted by the mucus-secreting pit cells of the corpus and antropyloric regions of the stomach. TFF1 has been reported as a gastric-specific tumour-suppressor gene. The TFF1 protein, which is secreted as a component of the protective mucus layer in the stomach, is highly expressed in response to mucosal injury. Previous reports have shown loss of TFF1 protein expression in more than two-thirds of gastric adenocarcinomas (AC) because of mutation-independent mechanisms. The silencing of the TFF1 gene expression in gastric cancer is predominantly induced by the loss of heterozygosity and hypermethylation of the TFF1 promoter. The TFF1-knockout mouse model provided first evidence supporting a tumor suppressor role of TFF1 in gastric tumorigenesis, demonstrating that it is essential for normal differentiation of the antral and pyloric gastric mucosa. The NF-κB transcription factor, regulated via the IκB kinase (IKK) complex, play a critical role in coupling inflammation and cancer. The activation of the NF-κB signaling pathway promotes the induction of inflammation-associated tumors and suppresses apoptosis in advanced tumors. In the regulation of the complex cancer-inducing NF-κB signaling, TFF1 plays an important role in NF-κB-mediated inflammatory response in the multistep gastric tumorigenesis cascade.

In principle, protein-based therapeutics offer to a way to control biochemical processes in living cells under non steady-state conditions and with fewer off target effects than conventional small molecule therapeutics. In practice, systemic protein delivery in animals has proven difficult due to poor tissue penetration and rapid clearance. Protein transduction exploits the ability of some cell-penetrating peptide (CPP) sequences to enhance the uptake of proteins and other macromolecules by mammalian cells. Previously developed hydrophobic CPPs, named membrane translocating sequence (MTS), membrane translocating motif (MTM) and macromolecule transduction domain (MTD), are able to deliver biologically active proteins into a variety of cells and tissues. Various cargo proteins fused to these CPPs have been used to test the functional and/or therapeutic efficacy of protein transduction. However, the recombinant proteins fused to previously develop hydrophobic CPPs displayed extremely low solubility, poor yields and relatively low cell- and tissue-permeability. Therefore, these recombinant proteins were not suitable for further clinical development as therapeutic agents. To overcome these limitations, cell-permeable TFF1 recombinant proteins (CP-TFF1) fused to the combination of novel hydrophobic CPPs, namely advanced macromolecule transduction domains (aMTDs) to greatly improve the efficiency of membrane penetrating ability in vitro and in vivo with solubilization domains to increase in their solubility and manufacturing yield when expressed and purified from bacteria cells.

We have hypothesized that exogenously administered TFF1 proteins can compensate for the apparent inability of endogenously expressed members of this physiologic tumor suppressor in GIT to cure the established gastric cancers. We have tried to demonstrate that this approach namely “intracellular protein therapy” by designing and introducing cell-permeable form of TFF1 to determine its potential of anti-cancer therapeutic applicability. The present invention suggests that intracellular restoration of TFF1 with CP-TFF1 creates a new paradigm for anti-cancer therapy, and the intracellular protein replacement therapy with the TFF1 recombinant protein fused to the combination of aMTD and SDs pair may be useful to treat the cancer.

### SUMMARY

An aspect of the present invention relates to cell-permeable TFF1 (CP-TFF1) recombinant proteins capable of mediating the transduction of biologically active macromolecules into live cells.

CP-TFF1 fused to novel hydrophobic CPPs—namely advanced macromolecule transduction domains (aMTDs)—greatly improve the efficiency of membrane penetrating ability in vitro and in vivo of the recombinant proteins.

CP-TFF1 fused to solubilization domains (SDs) greatly increase in their solubility and manufacturing yield when they are expressed and purified in the bacteria system.

The present invention also, relates to its therapeutic application for delivery of a biologically active molecule to a cell, involving a cell-permeable TFF1 recombinant protein, where the aMTD is attached to a biologically active cargo molecule.

Other aspects of the present invention relate to the development of TFF1 recombinant protein fused with aMTD sequences for drug delivery, protein therapy, intracellular protein therapy, protein replacement therapy and peptide therapy.

An aspect of the present invention provides cell-permeable TFF1 as a biotherapeutics having improved solubility/yield and cell-/tissue-permeability and anti-gastric cancer effects. Therefore, this would allow their practically effective applications in drug delivery and protein therapy including intracellular protein therapy and protein replacement therapy.

### BRIEF DESCRIPTION OF DRAWINGS

The above and other objects, features and other advantages of the present invention will be more clearly understood from the following detailed description taken in conjunction with the accompanying drawings.

FIG. 1 shows that the determination of aMTD43- and aMTD165-Mediated cell-permeability. uptake of aMTD-EGFP fusion proteins by RAW264.7 cells. Cells were exposed to the FITC conjugated proteins (10 μM) for 1 hour, treated to remove cell-associated but non-internalized protein and analyzed by flow cytometry. The EGFP protein cargos contained aMTDs (red), a SDA only (blue), FITC only (green), or vehicle (shaded). Lower Panel: EGFP protein uptake by NIH3T3 cells. Cells were visualized by fluorescence confocal laser scanning microscopy.

FIG. 2 shows that the determination of aMTD43- and aMTD165-mediated intracellular delivery and localization. Uptake of aMTD-EGFP fusion proteins by NIH3T3 cells. Cells were exposed to the FITC conjugated proteins (10 μM) for 1 hour, treated to remove cell-associated but non-internalized protein. Cells were visualized by fluorescence confocal laser scanning microscopy.

FIG. 3 shows that the structure of aMTD/SD-fused TFF1 recombinant proteins: Set 1. A schematic Diagram of aMTD/SD-fused TFF1 recombinant proteins containing, aMTD43 and SDA or SDB is illustrated and constructed (Set 1).

FIG. 4 shows that the inducible expression and purification of TFF1 recombinant proteins: Set 1. Proteins expressed in E. coli were determined by SDS-PAGE analysis of cell lysates before (−) and after (+) induction with IPTG; aliquots of Ni2+ affinity purified proteins (P); and size marker (M). Solubility was scored a 5-point scale ranging from highly soluble proteins with little tendency to precipitate (+++++) to largely insoluble proteins (+).

FIG. 5 shows that the structural change of aMTD/SD-fused TFF1 recombinant protein: Set 2. A schematic Diagram of aMTD/SD-fused TFF1 recombinant proteins containing, aMTD165 and SDC or SDD is illustrated and constructed (Set 2).

FIG. 6 shows that the inducible expression and purification of TFF1 recombinant proteins: Set 2. Proteins expressed in E. coli were determined by SDS-PAGE analysis of cell lysates before (−) and after (+) induction with IPTG; aliquots of Ni2+ affinity purified proteins (P); and size marker (M). Solubility was scored a 5-point scale ranging from highly soluble proteins with little tendency to precipitate (+++++) to largely insoluble proteins (+).

FIG. 7 shows that the structure of aMTD/SD-fused TFF1 recombinant protein: Set 3. A schematic Diagram of aMTD/SD-fused TFF1 recombinant proteins containing, aMTD165 and SDA and SDB is illustrated and constructed (Set 3).

FIG. 8 shows that the inducible expression and purification of TFF1 recombinant proteins: Set 3. Proteins expressed in E. coli were determined by SDS-PAGE analysis of cell lysates before (−) and after (+) induction with IPTG; aliquots of Ni2+ affinity purified proteins (P); and size marker (M). Solubility was scored a 5-point scale ranging from highly soluble proteins with little tendency to precipitate (+++++) to largely insoluble proteins (+).

FIG. 9 shows that aMTD-mediated cell-permeability of TFF1 recombinant proteins. RAW264.7 cells were exposed to 10 μM of the FITC conjugated TFF1 proteins containing aMTD165 and solubilization domains, lacking aMTD (HST1SB, HSsT1SB) or 10 μM of FITC alone (green) for 1 hour and analyzed by flow cytometry.

FIG. 10 shows that aMTD-mediated intracellular delivery and localization of recombinant proteins. NIH3T3 cells were exposed to 10 μM of the FITC conjugated TFF1 proteins containing aMTD165 and solubilization domains (HSAM165T1SB), lacking aMTD (HSAT1SB, HST1SB), lacking cargo (HSAM165SB) or 10 μM of FITC alone (green) for 1 hour and visualized by fluorescence confocal laser scanning microscopy.

FIG. 11 shows that the systemic delivery of aMTD/SD-fused TFF1 recombinant proteins In vivo. CP-TFF1 recombinant proteins were systemically delivered to various tissues. Tissue distribution of the recombinant proteins (green staining) was assessed by fluorescence microscopy.

FIG. 12 shows that the mechanism of aMTD-mediated TFF1 proteins uptake into cells. (A) Cell surface protein-independence of aMTD165-mediated protein uptake. (B) Endocytosis-independence of aMTD165-mediated protein uptake. (C) ATP source-independence of aMTD165-mediated protein uptake. (D) EDTA suppresses aMTD165-mediated protein uptake. (E) Temperature-dependence of aMTD165-stimulated protein uptake. Cells (shaded) were exposed for one hour to HSAM165T1SB (red), HSAT1SB (blue) and FITC (green) were processed as before to remove non-internalized protein and were analyzed by flow cytometry.

FIG. 13 shows that the cell-permeability of CP-TFF1 (HSAM165T1SB) in gastric cancer cells. Gastric cell lines (AGS and MKN75 cells) were exposed to 10 μM of the FITC conjugated TFF1 proteins containing aMTD165 and solubilization domains, lacking aMTD (HST1SB, HST1SB) or 10 μM of FITC alone (green) for 1 hour and analyzed by flow cytometry.

FIG. 14 shows that the tissue distribution of CP-TFF1 into stomach. Systemic TFF1 recombinant protein delivery to murine stomach. Tissue distribution of the recombinant proteins (green staining) was assessed by fluorescence microscopy.

FIG. 15 shows that the CP-TFF1 inhibits proliferation of gastric cancer cells. Various gastric cancer cells (AGS, MKN45 and NCI-N87 Cells) were treated with CP-TFF1 (HSAM165T1SB, 10 μM) for 72 hours. Cell viability assay was evaluated with Cell-Titer Glo luminescent cell viability assay. All experimental data obtained using cultured cells were expressed as means S.D. For the ATP-Glo cell viability assay, statistical significance was evaluated using a one-tailed Student t-test. Statistical significance was established at p<0.05.

FIGS. 16A to 16C show that the CP-TFF1 inhibits migration of gastric cancer cells. Representative photomicrographs of wound healing assays. After gastric cancer cell lines (AGS, MKN45 and STKM2 cells) were seeded into 12-well plates and grown to 90% confluence, wounds were made by scraping the cell layer with a sterile tip. Cells were treated with CP-TFF1 (HSAM165T1SB, 10 μM) for 2 hours before changing the growth medium. Photograph was taken after 24 hours of incubation. *: p<0.05.

FIG. 17 shows that the CP-TFF1 Inhibits transwell migration of gastric cancer cells. Representative photomicrographs of Transwell migration assay. Gastric cancer cells, AGS were treated with CP-TFF1 (HSAM165T1SB, 10 μM) proteins for 24 hour in serum-free media, and migration were measured by Transwell assay. Photograph was taken after 24 hours of incubation. *: p<0.05.

FIG. 18 shows that the CP-TFF1 induces expression of apoptosis-related protein in human gastric cancer cells. AGS and NCI-N87 cells were treated for 24 hours with 10 μM CP-TFF1 recombinant proteins. Equal amounts of cell lysate protein were immunoblotted with cleaved Caspase-3 antibody. β-actin was used for loading control.

FIG. 19 shows that the external appearances of human gastric tumor bearing mice. Tumor was induced by implanted with MKN45 tumor block. After tumor reached a size of 50-80 mm3 (start), the mice were injected daily (I.V.) for 3 weeks with the diluent alone, HSAT1SB (TFF1) or HSAM165T1SB (CP-TFF1) and observed for 3 weeks following the termination of the treatment.

FIG. 20 shows that the CP-TFF1 suppresses tumor growth in mouse xenograft model. Tumor was induced by implanted with MKN45 tumor block. After tumor reached a size of 50-80 mm3 (start), the mice were injected daily (I.V.) for 3 weeks with the diluent alone, HSAT1SB (TFF1) or HSAM165T1SB (CP-TFF1) and observed for 3 weeks following the termination of the treatment. *P<0.05 as determined by the Student t-test.

FIG. 21 shows that the CP-TFF1 inhibits tumor growth in xenograft model. Tumor was induced by implanted with MKN45 tumor block. After tumors reached a size of 50 to 80 m3 (start), mouse were injected daily (I. V.) for 3 weeks with diluent alone (black), 800 μg TFF1 (blue) or CP-TFF1 (red). Tumor growth was suppressed to varying degrees after the protein therapy ended (stop). *P<0.05 as determined by the Student t-test.

FIG. 22 shows that the CP-TFF1 regulates expression of tumor-associated proteins in human Tumor Xenograft. Tumor was induced by implanted with MKN45 tumor block. After tumors reached a size of 50 to 80 m3 (start), mouse were injected daily (I. V.) for 3 weeks with diluent alone, 800 μg TFF1 or CP-TFF1. Tumor tissues from mice treated daily for 3 weeks with indicated proteins and observed for 3 weeks following the termination of the treatment were sectioned and immunostained with antibodies against VEGF.

### DETAILED DESCRIPTION

In this invention, it has been hypothesized that exogenously administered TFF1 proteins can compensate for the apparent inability of endogenously expressed members of this physiologic tumor suppressor in GIT to cure the established gastric cancers. To prove our hypothesis, the TFF1 recombinant proteins fused to novel hydrophobic CPPs called aMTDs to improve their cell-/tissue-permeability and additionally adopted solubilization domains to increase their solubility/yield in physiological condition, and then tested whether exogenous administration of TFF1 proteins can reconstitute their endogenous stores and restore their basic function as the tumor suppressor. This art of invention has demonstrated “intracellular protein therapy” by designing and introducing cell-permeable form of TFF1 has a great potential of anti-cancer therapeutic applicability in gastric cancer.

### 1. Novel Hydrophobic Cell-Penetrating Peptides—Advanced Macromolecule Transduction Domains

### 1-1. Analysis of Hydrophobic CPP

To address the limitation of previously developed hydrophobic CPPs, novel sequences have been developed. To design new hydrophobic CPPs for intracellular delivery of cargo proteins such as TFFF1, identification of optimal common sequence and/or homologous structural determinants, namely critical factors (CFs), had been crucial. To do it, the physicochemical characteristics of previously published hydrophobic CPPs were analyzed. To keep the similar mechanism on cellular uptake, all CPPs analyzed were hydrophobic region of signal peptide (HRSP)-derived CPPs (e.g. membrane translocating sequence: MTS and macromolecule transduction domain: MTD) as explained previously.

### (1) Basic Characteristics of CPPs Sequence.

These 17 hydrophobic CPPs published from 1995 to 2014 have been analyzed for their 11 different characteristics—sequence, amino acid length, molecular weight, pl value, bending potential, rigidity/flexibility, structural feature, hydropathy, residue structure, amino acid composition, and secondary structure of the sequences. Two peptide/protein analysis programs were used (ExPasy: http://web.expasy.org/protparam/, SoSui: http://harrier.nagahama-i-bio.ac.jp/sosui/sosui_submit.html) to determine various indexes, structural features of the peptide sequences and to design new sequence. Followings are important factors analyzed.

Average length, molecular weight and pl value of the peptides analyzed were 10.8±2.4, 1,011±189.6 and 5.6±0.1, respectively.

TABLE 4
[Characteristics of aMTD43 and aMTD165]

| | | | Rigidity/ | Structural | Hydrop- |
|---|---|---|---|---|---|
| | | Length | Flexibility | Feature | athy |
| No. | Sequences | (a.a) | (II) | (AI) | (GRAVY) |
| 43 | LLAPLVVAAVP | 12 | 41.2 | 203.3 | 2.3 |
| 165 | ALAVPVALAIVP | 12 | 50.2 | 203.3 | 2.3 |

### (2) Bending Potential (Proline Position: PP)

Bending potential (Bending or No-Bending) was determined based on the fact whether proline (P) exists and/or where the amino acid(s) providing bending potential to the peptide in recombinant protein is/are located. Proline differs from the other common amino acids in that its side chain is bonded to the backbone nitrogen atom as well as the alpha-carbon atom. The resulting cyclic structure markedly influences protein architecture which is often found in the bends of folded peptide/protein chain. Eleven out of 17 were determined as ‘Bending’ peptide which means that proline should be present in the middle of sequence for peptide bending and/or located at the end of the peptide for protein bending. As indicated above, peptide sequences could penetrate the plasma membrane in a “bent” configuration. Therefore, bending or no-bending potential is considered as one of the critical factors for the improvement of current hydrophobic CPPs.

### (3) Rigidity/Flexibility (Instability Index: II)

Since one of the crucial structural features of any peptide is based on the fact whether the motif is rigid or flexible, which is an intact physicochemical characteristic of the peptide sequence, instability index (II) of the sequence was determined. The index value representing rigidity/flexibility of the peptide was extremely varied (8.9−79.1), but average value was 40.1±21.9 which suggested that the peptide should be somehow flexible, but not too rigid or flexible.

### (4) Hydropathy (Grand Average of Hydropathy: GRAVY) and Structural Feature (Aliphatic Index: AI)

Alanine (V), valine (V), leucine (L) and isoleucine (I) contain aliphatic side chain and are hydrophobic—that is, they have an aversion to water and like to cluster. These amino acids having hydrophobicity and aliphatic residue enable them to pack together to form compact structure with few holes. Analyzed peptide sequence showed that all composing amino acids were hydrophobic (A, V, L and I) except glycine (G) in only one out of 17 and aliphatic (A, V, L, I, and P). Their hydropathic index (Grand Average of Hydropathy: GRAVY) and aliphatic index (Al) were 2.5±0.4 and 217.9±43.6, respectively.

### (5) Secondary Structure (Cc-Helix)

As explained above, the CPP sequences may be supposed to penetrate the plasma membrane directly after inserting into the membranes in a “bent” configuration with hydrophobic sequences adopting an α-helical conformation. In addition, our analysis strongly indicated that bending potential was crucial. Therefore, structural analysis of the peptides conducted to determine whether the sequence was to form helix or not. Nine peptides were helix and 8 were not. It seems to suggest that helix structure may not be required, but favored for membrane penetration.

### (6) Determination of Critical Factors (CFs)

In the 11 characteristics analyzed, the following 6 are selected namely “Critical Factors (CFs)” for the development of new hydrophobic CPPs—advanced MTDs: i) amino acid length, ii) bending potential (proline presence and location), iii) rigidity/flexibility (instability index: II), iv) structural feature (aliphatic index: AI), v) hydropathy (GRAVY) and vi) amino acid composition/residue structure (hydrophobic and aliphatic A/a).

### 1-2. Analysis of Selected Hydrophobic CPPs to Optimize ‘Critical Factors’

Since the analyzed data of the 17 different hydrophobic CPPs (analysis A) previously developed during the past 2 decades showed high variation and were hard to make common- or consensus-features, additional analysis B and C was also conducted to optimize the critical factors for better design of improved CPPs-aMTDs.

In analysis B, 8 CPPs were used with each cargo in vivo. Length was 11±3.2, but 3 out of 8 CPPs possessed little bending potential. Rigidity/Flexibility was 41±15, but removing one [MTD85: rigid, with minimal (II: 9.1)] of the peptides increased the overall instability index to 45.6±9.3. This suggested that higher flexibility (40 or higher II) is potentially be better. All other characteristics of the 8 CPPs were similar to the analysis A, including structural feature and hydropathy.

To optimize the ‘Common Range and/or Consensus Feature of Critical Factor’ for the practical design of aMTDs and the random peptides (rPs or rPeptides), which were to prove that the ‘Critical Factors’ determined in the analysis A, B and C were correct to improve the current problems of hydrophobic CPPs—protein aggregation, low solubility/yield, and poor cell/tissue-permeability of the recombinant proteins fused to the MTS/MTM or MTD, and non-common sequence and non-homologous structure of the peptides, empirically selected peptides were analyzed for their structural features and physicochemical factor indexes.

The peptides which did not have a bending potential, rigid or too flexible sequences (too low or too high Instability Index), or too low or too high hydrophobic CPP were unselected, but secondary structure was not considered because helix structure of sequence was not required. 8 selected CPP sequences that could provide a bending potential and higher flexibility were finally analyzed. Common amino acid length is 12 (11.6±3.0). Proline should be presence in the middle of and/or the end of sequence. Rigidity/Flexibility (II) is 45.5−57.3 (Avg: 50.1±3.6). Al and GRAVY representing structural feature and hydrophobicity of the peptide are 204.7±37.5 and 2.4±0.3, respectively. All peptides are consisted with hydrophobic and aliphatic amino acids (A, V, L, I, and P). Therefore, analysis C was chosen as a standard for the new design of new hydrophobic CPPs (TABLE 1).

1. Amino Acid Length: 9-13

2. Bending Potential (Proline Position: PP)

: Proline presences in the middle (from 5′ to 8′ amino acid) and at the end of sequence

3. Rigidity/Flexibility (Instability Index: II): 40-60

4. Structural Feature (Aliphatic Index: AI): 180-220

5. Hydropathy (GRAVY): 2.1-2.6

6. Amino Acid Composition: Hydrophobic and Aliphatic amino acids—A, V, L, I and P

TABLE 1
[Universal Structure of Newly Developed Hydrophobic CPPs]
Summarized Critical Factors of aMTD

| | Newly Designed CPPs |
|---|---|
| Critical Factor | Range |
| Bending Potential | Proline presences in the |
| (Proline Position: PP) | middle (5′, 6′, 7′ or 8′) and |
| | at the end (12′) of peptides |
| Rigidity/Flexibility | 40-60 |
| (Instability Index: II) | |
| Structural Feature | 180-220 |
| (Aliphatic Index: AI) | |
| Hydropathy | 2.1-2.6 |
| (Grand Average of | |
| Hydropathy GRAVY) | |
| Length | 9-13 |
| (Number of Amino Acid) | |
| Amino acid Composition | A, V, I, L, P |

1-3. Determination of Critical Factors for Development of aMTDs

For confirming the validity of 6 critical factors providing the optimized cell-/tissue-permeability, 240 aMTD sequences have been designed and developed based on six critical factors (TABLES 2-1 to 2-6 and 3). All 240 aMTDs (hydrophobic, flexible, bending, aliphatic and helical 12 a/a-length peptides) are practically confirmed by their quantitative and visual cell-permeability. To determine the cell-permeability of aMTDs and random peptides, which do not satisfy one or more critical factors have also been designed and tested. Relative cell-permeability of 240 aMTDs to the negative control (random peptide) was significantly increased by up to 164 fold, with average increase of 19.6±1.6. Moreover, novel 240 aMTDs averaged of 13±1.1 (maximum 109.9) and 6.6±0.5 (maximum 55.5) fold higher cell-permeability, respectively. As a result, there were vivid association of cell-permeability of the peptides and critical factors. According to the result from the newly designed and tested novel 240 aMTDs, the empirically optimized critical factors (CFs) are provided below.

1. Amino Acid Length: 12

2. Bending Potential (Proline Position: PP)

: Proline presences in the middle (from 5′ to 8′ amino acid) and at the end of sequence

3. Rigidity/Flexibility (Instability Index: II): 41.3-57.3

4. Structural Feature (Aliphatic Index: AI): 187.5-220.0

5. Hydropathy (GRAVY): 2.2-2.6

6. Amino Acid Composition: Hydrophobic and Aliphatic amino acids—A, V, L, I and P

TABLE 2-1
[Newly Developed Hydrophobic CPPs - 240 aMTDs That All Critical Factors Are
Considered and Satisfied (Sequence ID No 1-46)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 1 | 1 | AAALAPVVLALP | 12 | 57.3 | 187.5 | 2.1 | Aliphatic |
| 2 | 2 | AAAVPLLAVVVP | 12 | 41.3 | 195.0 | 2.4 | Aliphatic |
| 3 | 3 | AALLVPAAVLAP | 12 | 57.3 | 187.5 | 2.1 | Aliphatic |
| 4 | 4 | ALALLPVAALAP | 12 | 57.3 | 195.8 | 2.1 | Aliphatic |
| 5 | 5 | AAALLPVALVAP | 12 | 57.3 | 187.5 | 2.1 | Aliphatic |
| 6 | 11 | VVALAPALAALP | 12 | 57.3 | 187.5 | 2.1 | Aliphatic |
| 7 | 12 | LLAAVPAVLLAP | 12 | 57.3 | 211.7 | 2.3 | Aliphatic |
| 8 | 13 | AAALVPVVALLP | 12 | 57.3 | 203.3 | 2.3 | Aliphatic |
| 9 | 21 | AVALLPALLAVP | 12 | 57.3 | 211.7 | 2.3 | Aliphatic |
| 10 | 22 | AVVLVPVLAAAP | 12 | 57.3 | 195.0 | 2.4 | Aliphatic |
| 11 | 23 | VVLVLPAAAAVP | 12 | 57.3 | 195.0 | 2.4 | Aliphatic |
| 12 | 24 | IALAAPALIVAP | 12 | 50.2 | 195.8 | 2.2 | Aliphatic |
| 13 | 25 | IVAVAPALVALP | 12 | 50.2 | 203.3 | 2.4 | Aliphatic |
| 14 | 42 | VAALPVVAVVAP | 12 | 57.3 | 186.7 | 2.4 | Aliphatic |
| 15 | 43 | LLAAPLVVAAVP | 12 | 41.3 | 187.5 | 2.1 | Aliphatic |
| 16 | 44 | ALAVPVALLVAP | 12 | 57.3 | 203.3 | 2.3 | Aliphatic |
| 17 | 61 | VAALPVLLAALP | 12 | 57.3 | 211.7 | 2.3 | Aliphatic |
| 18 | 62 | VALLAPVALAVP | 12 | 57.3 | 203.3 | 2.3 | Aliphatic |
| 19 | 63 | AALLVPALVAVP | 12 | 57.3 | 203.3 | 2.3 | Aliphatic |
| 20 | 64 | AIVALPVAVLAP | 12 | 50.2 | 203.3 | 2.4 | Aliphatic |
| 21 | 65 | IAIVAPVVALAP | 12 | 50.2 | 283.3 | 2.4 | Aliphatic |
| 22 | 81 | AALLPALAALLP | 12 | 57.3 | 204.2 | 2.1 | Aliphatic |
| 23 | 82 | AVVLAPVAAVLP | 12 | 57.3 | 195.0 | 2.4 | Aliphatic |
| 24 | 83 | LAVAAPLALALP | 12 | 41.3 | 195.8 | 2.1 | Aliphatic |
| 25 | 84 | AAVAAPLLLALP | 12 | 41.3 | 195.8 | 2.1 | Aliphatic |
| 26 | 85 | LLVLPAAALAAP | 12 | 57.3 | 195.8 | 2.1 | Aliphatic |
| 27 | 101 | LVALAPVAAVLP | 12 | 57.3 | 203.3 | 2.3 | Aliphatic |
| 28 | 102 | LALAPAALALLP | 12 | 57.3 | 204.2 | 2.1 | Aliphatic |
| 29 | 103 | ALIAAPILALAP | 12 | 57.3 | 204.2 | 2.2 | Aliphatic |
| 30 | 104 | AVVAAPLVLALP | 12 | 41.3 | 203.3 | 2.3 | Aliphatic |
| 31 | 105 | LLALAPAALLAP | 12 | 57.3 | 204.1 | 2.1 | Aliphatic |
| 32 | 121 | AIVALPALALAP | 12 | 50.2 | 195.8 | 2.2 | Aliphatic |
| 33 | 123 | AAIIVPAALLAP | 12 | 50.2 | 195.8 | 2.2 | Aliphatic |
| 34 | 124 | IAVALPALIAAP | 12 | 50.3 | 195.8 | 2.2 | Aliphatic |
| 35 | 141 | AVIVLPALAVAP | 12 | 50.2 | 203.3 | 2.4 | Aliphatic |
| 36 | 143 | AVLAVPAVLVAP | 12 | 57.3 | 195.0 | 2.4 | Aliphatic |
| 37 | 144 | VLAIVPAVALAP | 12 | 50.2 | 203.3 | 2.4 | Aliphatic |
| 38 | 145 | LLAVVPAVALAP | 12 | 57.3 | 203.3 | 2.3 | Aliphatic |
| 39 | 161 | AVIALPALIAAP | 12 | 57.3 | 195.8 | 2.2 | Aliphatic |
| 40 | 162 | AVVALPAALIVP | 12 | 50.2 | 203.3 | 2.4 | Aliphatic |
| 41 | 163 | LALVLPAALAAP | 12 | 57.3 | 195.8 | 2.1 | Aliphatic |
| 42 | 164 | LAAVLPALLAAP | 12 | 57.3 | 195.8 | 2.1 | Aliphatic |
| 43 | 165 | ALAVPVALAIVP | 12 | 50.2 | 203.3 | 2.4 | Aliphatic |
| 44 | 182 | ALIAPVVALVAP | 12 | 57.3 | 203.3 | 2.4 | Aliphatic |
| 45 | 183 | LLAAPVVIALAP | 12 | 57.3 | 211.6 | 2.4 | Aliphatic |
| 46 | 184 | LAAIVPAIIAVP | 12 | 50.2 | 211.6 | 2.4 | Aliphatic |

TABLE 2-2
[Newly Developed Hydrophobic CPPs - 240 aMTDs That All Critical Factors Are
Considered and Satisfied (Sequence ID No 47-92)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 47 | 185 | AALVLPLIIAAP | 12 | 41.3 | 220.0 | 2.4 | Aliphatic |
| 48 | 201 | LALAVPALAALP | 12 | 57.3 | 195.8 | 2.1 | Aliphatic |
| 49 | 204 | LIAALPAVAALP | 12 | 57.3 | 195.8 | 2.2 | Aliphatic |
| 50 | 205 | ALALVPAIAALP | 12 | 57.3 | 195.8 | 2.2 | Aliphatic |
| 51 | 221 | AAILAPIVALAP | 12 | 50.2 | 195.8 | 2.2 | Aliphatic |
| 52 | 222 | ALLIAPAAVIAP | 12 | 57.3 | 195.8 | 2.2 | Aliphatic |
| 53 | 223 | AILAVPIAVVAP | 12 | 57.3 | 203.3 | 2.4 | Aliphatic |
| 54 | 224 | ILAAVPIALAAP | 12 | 57.3 | 195.8 | 2.2 | Aliphatic |
| 55 | 225 | VAALLPAAAVLP | 12 | 57.3 | 187.5 | 2.1 | Aliphatic |
| 56 | 241 | AAAVVPVLLVAP | 12 | 57.3 | 195.0 | 2.4 | Aliphatic |
| 57 | 242 | AALLVPALVAAP | 12 | 57.3 | 187.5 | 2.1 | Aliphatic |
| 58 | 243 | AAVLLPVALAAP | 12 | 57.3 | 187.5 | 2.1 | Aliphatic |
| 59 | 245 | AAALAPVLALVP | 12 | 57.3 | 187.5 | 2.1 | Aliphatic |
| 60 | 261 | LVLVPLLAAAAP | 12 | 41.3 | 211.6 | 2.3 | Aliphatic |
| 61 | 262 | ALIAVPAIIVAP | 12 | 50.2 | 211.6 | 2.4 | Aliphatic |
| 62 | 263 | ALAVIPAAAILP | 12 | 54.9 | 195.8 | 2.2 | Aliphatic |
| 63 | 264 | LAAAPVVIVIAP | 12 | 50.2 | 203.3 | 2.4 | Aliphatic |
| 64 | 265 | VLAIAPLLAAVP | 12 | 41.3 | 211.6 | 2.3 | Aliphatic |
| 65 | 281 | ALIVLPAAVAVP | 12 | 50.2 | 203.3 | 2.4 | Aliphatic |
| 66 | 282 | VLAVAPALIVAP | 12 | 50.2 | 203.3 | 2.4 | Aliphatic |
| 67 | 283 | AALLAPALIVAP | 12 | 50.2 | 195.8 | 2.2 | Aliphatic |
| 68 | 284 | ALIAPAVALIVP | 12 | 50.2 | 211.7 | 2.4 | Aliphatic |
| 69 | 285 | AIVLLPAAVVAP | 12 | 50.2 | 203.3 | 2.4 | Aliphatic |
| 70 | 301 | VIAAPVLAVLAP | 12 | 57.3 | 203.3 | 2.4 | Aliphatic |
| 71 | 302 | LALAPALALLAP | 12 | 57.3 | 204.2 | 2.1 | Aliphatic |
| 72 | 304 | AIILAPIAAIAP | 12 | 57.3 | 204.2 | 2.3 | Aliphatic |
| 73 | 305 | IALAAPILLAAP | 12 | 57.3 | 204.2 | 2.2 | Aliphatic |
| 74 | 321 | IVAVALPALAVP | 12 | 50.2 | 203.3 | 2.3 | Aliphatic |
| 75 | 322 | VVAIVLPALAAP | 12 | 50.2 | 203.3 | 2.3 | Aliphatic |
| 76 | 323 | IVAVALPVALAP | 12 | 50.2 | 203.3 | 2.3 | Aliphatic |
| 77 | 324 | IVAVALPAALVP | 12 | 50.2 | 203.3 | 2.3 | Aliphatic |
| 78 | 325 | IVAVALPAVALP | 12 | 50.2 | 203.3 | 2.3 | Aliphatic |
| 79 | 341 | IVAVALPAVLAP | 12 | 50.2 | 203.3 | 2.3 | Aliphatic |
| 80 | 342 | VIVALAPAVLAP | 12 | 50.2 | 203.3 | 2.3 | Aliphatic |
| 81 | 343 | IVAVALPALVAP | 12 | 50.2 | 203.3 | 2.3 | Aliphatic |
| 82 | 345 | ALLIVAPVAVAP | 12 | 50.2 | 203.3 | 2.3 | Aliphatic |
| 83 | 361 | AVVIVAPAVIAP | 12 | 50.2 | 195.0 | 2.4 | Aliphatic |
| 84 | 363 | AVLAVAPALIVP | 12 | 50.2 | 203.3 | 2.3 | Aliphatic |
| 85 | 364 | LVAAVAPALIVP | 12 | 50.2 | 203.3 | 2.3 | Aliphatic |
| 86 | 365 | AVIVVAPALLAP | 12 | 50.2 | 203.3 | 2.3 | Aliphatic |
| 87 | 381 | VVAIVLPAVAAP | 12 | 50.2 | 195.0 | 2.4 | Aliphatic |
| 88 | 382 | AAALVIPAILAP | 12 | 54.9 | 195.8 | 2.2 | Aliphatic |
| 89 | 383 | VIVALAPALLAP | 12 | 50.2 | 211.6 | 2.3 | Aliphatic |
| 90 | 384 | VIVAIAPALLAP | 12 | 50.2 | 211.6 | 2.4 | Aliphatic |
| 91 | 385 | IVAIAVPALVAP | 12 | 50.2 | 203.3 | 2.4 | Aliphatic |
| 92 | 401 | AALAVIPAAILP | 12 | 54.9 | 195.8 | 2.2 | Aliphatic |

TABLE 2-3
[Newly Developed Hydrophobic CPPs - 240 aMTDs That All Critical Factors Are
Considered and Satisfied (Sequence ID No 93-138)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 93 | 402 | ALAAVIPAAILP | 12 | 54.9 | 195.8 | 2.2 | Aliphatic |
| 94 | 403 | AAALVIPAAILP | 12 | 54.9 | 195.8 | 2.2 | Aliphatic |
| 95 | 404 | LAAAVIPAAILP | 12 | 54.9 | 195.8 | 2.2 | Aliphatic |
| 96 | 405 | LAAAVIPVAILP | 12 | 54.9 | 211.7 | 2.4 | Aliphatic |
| 97 | 421 | AAILAAPLIAVP | 12 | 57.3 | 195.8 | 2.2 | Aliphatic |
| 98 | 422 | VVAILAPLLAAP | 12 | 57.3 | 211.7 | 2.4 | Aliphatic |
| 99 | 424 | AVVVAAPVLALP | 12 | 57.3 | 195.0 | 2.4 | Aliphatic |
| 100 | 425 | AVVAIAPVLALP | 12 | 57.3 | 203.3 | 2.4 | Aliphatic |
| 101 | 442 | ALAALVPAVLVP | 12 | 57.3 | 203.3 | 2.3 | Aliphatic |
| 102 | 443 | ALAALVPVALVP | 12 | 57.3 | 203.3 | 2.3 | Aliphatic |
| 103 | 444 | LAAALVPVALVP | 12 | 57.3 | 203.3 | 2.3 | Aliphatic |
| 104 | 445 | ALAALVPALVVP | 12 | 57.3 | 203.3 | 2.3 | Aliphatic |
| 105 | 461 | IAAVIVPAVALP | 12 | 50.2 | 203.3 | 2.4 | Aliphatic |
| 106 | 462 | IAAVLVPAVALP | 12 | 57.3 | 203.3 | 2.4 | Aliphatic |
| 107 | 463 | AVAILVPLLAAP | 12 | 57.3 | 211.7 | 2.4 | Aliphatic |
| 108 | 464 | AVVILVPLAAAP | 12 | 57.3 | 203.3 | 2.4 | Aliphatic |
| 109 | 465 | IAAVIVPVAALP | 12 | 50.2 | 203.3 | 2.4 | Aliphatic |
| 110 | 481 | AIAIAIVPVALP | 12 | 50.2 | 211.6 | 2.4 | Aliphatic |
| 111 | 482 | ILAVAAIPVAVP | 12 | 54.9 | 203.3 | 2.4 | Aliphatic |
| 112 | 483 | ILAAAIIPAALP | 12 | 54.9 | 204.1 | 2.2 | Aliphatic |
| 113 | 484 | LAVVLAAPAIVP | 12 | 50.2 | 203.3 | 2.4 | Aliphatic |
| 114 | 485 | AILAAIVPLAVP | 12 | 50.2 | 211.6 | 2.4 | Aliphatic |
| 115 | 501 | VIVALAVPALAP | 12 | 50.2 | 203.3 | 2.4 | Aliphatic |
| 116 | 502 | AIVALAVPVLAP | 12 | 50.2 | 203.3 | 2.4 | Aliphatic |
| 117 | 503 | AAIIIVLPAALP | 12 | 50.2 | 220.0 | 2.4 | Aliphatic |
| 118 | 504 | LIVALAVPALAP | 12 | 50.2 | 211.7 | 2.4 | Aliphatic |
| 119 | 505 | AIIIVIAPAAAP | 12 | 50.2 | 195.8 | 2.3 | Aliphatic |
| 120 | 521 | LAALIVVPAVAP | 12 | 50.2 | 203.3 | 2.4 | Aliphatic |
| 121 | 522 | ALLVIAVPAVAP | 12 | 57.3 | 203.3 | 2.4 | Aliphatic |
| 122 | 524 | AVALIVVPALAP | 12 | 50.2 | 203.3 | 2.4 | Aliphatic |
| 123 | 525 | ALAIVVAPVAVP | 12 | 50.2 | 195.0 | 2.4 | Aliphatic |
| 124 | 541 | LLALIIAPAAAP | 12 | 57.3 | 204.1 | 2.1 | Aliphatic |
| 125 | 542 | ALALIIVPAVAP | 12 | 50.2 | 211.6 | 2.4 | Aliphatic |
| 126 | 543 | LLAALIAPAALP | 12 | 57.3 | 204.1 | 2.1 | Aliphatic |
| 127 | 544 | IVALIVAPAAVP | 12 | 43.1 | 203.3 | 2.4 | Aliphatic |
| 128 | 545 | VVLVLAAPAAVP | 12 | 57.3 | 195.0 | 2.3 | Aliphatic |
| 129 | 561 | AAVAIVLPAVVP | 12 | 50.2 | 195.0 | 2.4 | Aliphatic |
| 130 | 562 | ALIAAIVPALVP | 12 | 50.2 | 211.7 | 2.4 | Aliphatic |
| 131 | 563 | ALAVIVVPALAP | 12 | 50.2 | 203.3 | 2.4 | Aliphatic |
| 132 | 564 | VAIALIVPALAP | 12 | 50.2 | 211.7 | 2.4 | Aliphatic |
| 133 | 565 | VAIVLVAPAVAP | 12 | 50.2 | 195.0 | 2.4 | Aliphatic |
| 134 | 582 | VAVALIVPALAP | 12 | 50.2 | 203.3 | 2.4 | Aliphatic |
| 135 | 583 | AVILALAPIVAP | 12 | 50.2 | 211.6 | 2.4 | Aliphatic |
| 136 | 585 | ALIVAIAPALVP | 12 | 50.2 | 211.6 | 2.4 | Aliphatic |
| 137 | 601 | AAILIAVPIAAP | 12 | 57.3 | 195.8 | 2.3 | Aliphatic |
| 138 | 602 | VIVALAAPVLAP | 12 | 50.2 | 203.3 | 2.4 | Aliphatic |

TABLE 2-4
[Newly Developed Hydrophobic CPPs - 240 aMTDs That All Critical Factors Are
Considered and Satisfied (Sequence ID No 139-184)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 139 | 603 | VLVALAAPVIAP | 12 | 57.3 | 203.3 | 2.4 | Aliphatic |
| 140 | 604 | VALIAVAPAVVP | 12 | 57.3 | 195.0 | 2.4 | Aliphatic |
| 141 | 605 | VIAAVLAPVAVP | 12 | 57.3 | 195.0 | 2.4 | Aliphatic |
| 142 | 622 | ALIVLAAPVAVP | 12 | 50.2 | 203.3 | 2.4 | Aliphatic |
| 143 | 623 | VAAAIALPAIVP | 12 | 50.2 | 187.5 | 2.3 | Aliphatic |
| 144 | 625 | ILAAAAAPLIVP | 12 | 50.2 | 195.8 | 2.2 | Aliphatic |
| 145 | 643 | LALVLAAPAIVP | 12 | 50.2 | 211.6 | 2.4 | Aliphatic |
| 146 | 645 | ALAVVALPAIVP | 12 | 50.2 | 203.3 | 2.4 | Aliphatic |
| 147 | 661 | AAILAPIVAALP | 12 | 50.2 | 195.8 | 2.2 | Aliphatic |
| 148 | 664 | ILIAIAIPAAAP | 12 | 54.9 | 204.1 | 2.3 | Aliphatic |
| 149 | 665 | LAIVLAAPVAVP | 12 | 50.2 | 203.3 | 2.3 | Aliphatic |
| 150 | 666 | AAIAIIAPAIVP | 12 | 50.2 | 195.8 | 2.3 | Aliphatic |
| 151 | 667 | LAVAIVAPALVP | 12 | 50.2 | 203.3 | 2.3 | Aliphatic |
| 152 | 683 | LAIVLAAPAVLP | 12 | 50.2 | 211.7 | 2.4 | Aliphatic |
| 153 | 684 | AAIVLALPAVLP | 12 | 50.2 | 211.7 | 2.4 | Aliphatic |
| 154 | 685 | ALLVAVLPAALP | 12 | 57.3 | 211.7 | 2.3 | Aliphatic |
| 155 | 686 | AALVAVLPVALP | 12 | 57.3 | 203.3 | 2.3 | Aliphatic |
| 156 | 687 | AILAVALPLLAP | 12 | 57.3 | 220.0 | 2.3 | Aliphatic |
| 157 | 703 | IVAVALVPALAP | 12 | 50.2 | 203.3 | 2.4 | Aliphatic |
| 158 | 705 | IVAVALLPALAP | 12 | 50.2 | 211.7 | 2.4 | Aliphatic |
| 159 | 706 | IVAVALLPAVAP | 12 | 50.2 | 203.3 | 2.4 | Aliphatic |
| 160 | 707 | IVALAVLPAVAP | 12 | 50.2 | 203.3 | 2.4 | Aliphatic |
| 161 | 724 | VAVLAVLPALAP | 12 | 57.3 | 203.3 | 2.3 | Aliphatic |
| 162 | 725 | IAVLAVAPAVLP | 12 | 57.3 | 203.3 | 2.3 | Aliphatic |
| 163 | 726 | LAVAIIAPAVAP | 12 | 57.3 | 187.5 | 2.2 | Aliphatic |
| 164 | 727 | VALAIALPAVLP | 12 | 57.3 | 211.6 | 2.3 | Aliphatic |
| 165 | 743 | AIAIALVPVALP | 12 | 57.3 | 211.6 | 2.4 | Aliphatic |
| 166 | 744 | AAVVIVAPVALP | 12 | 50.2 | 195.0 | 2.4 | Aliphatic |
| 167 | 746 | VAIIVVAPALAP | 12 | 50.2 | 203.3 | 2.4 | Aliphatic |
| 168 | 747 | VALLAIAPALAP | 12 | 57.3 | 195.8 | 2.2 | Aliphatic |
| 169 | 763 | VAVLIAVPALAP | 12 | 57.3 | 203.3 | 2.3 | Aliphatic |
| 170 | 764 | AVALAVLPAVVP | 12 | 57.3 | 195.0 | 2.3 | Aliphatic |
| 171 | 765 | AVALAVVPAVLP | 12 | 57.3 | 195.0 | 2.3 | Aliphatic |
| 172 | 766 | IVVIAVAPAVAP | 12 | 50.2 | 195.0 | 2.4 | Aliphatic |
| 173 | 767 | IVVAAVVPALAP | 12 | 50.2 | 195.0 | 2.4 | Aliphatic |
| 174 | 783 | IVALVPAVAIAP | 12 | 50.2 | 203.3 | 2.5 | Aliphatic |
| 175 | 784 | VAALPAVALVVP | 12 | 57.3 | 195.0 | 2.4 | Aliphatic |
| 176 | 786 | LVAIAPLAVLAP | 12 | 41.3 | 211.7 | 2.4 | Aliphatic |
| 177 | 787 | AVALVPVIVAAP | 12 | 50.2 | 195.0 | 2.4 | Aliphatic |
| 178 | 788 | AIAVAIAPVALP | 12 | 57.3 | 187.5 | 2.3 | Aliphatic |
| 179 | 803 | AIALAVPVLALP | 12 | 57.3 | 211.7 | 2.4 | Aliphatic |
| 180 | 805 | LVLIAAAPIALP | 12 | 41.3 | 220.0 | 2.4 | Aliphatic |
| 181 | 806 | LVALAVPAAVLP | 12 | 57.3 | 203.3 | 2.3 | Aliphatic |
| 182 | 807 | AVALAVPALVLP | 12 | 57.3 | 203.3 | 2.3 | Aliphatic |
| 183 | 808 | LVVLAAAPLAVP | 12 | 41.3 | 203.3 | 2.3 | Aliphatic |
| 184 | 809 | LIVLAAPALAAP | 12 | 50.2 | 195.8 | 2.2 | Aliphatic |

TABLE 2-5
[Newly Developed Hydrophobic CPPs - 240 aMTDs That All Critical Factors Are
Considered and Satisfied (Sequence ID No 185-230)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 185 | 810 | VIVLAAPALAAP | 12 | 50.2 | 187.5 | 2.2 | Aliphatic |
| 186 | 811 | AVVLAVPALAVP | 12 | 57.3 | 195.0 | 2.3 | Aliphatic |
| 187 | 824 | LIIVAAAPAVAP | 12 | 50.2 | 187.5 | 2.3 | Aliphatic |
| 188 | 825 | IVAVIVAPAVAP | 12 | 43.2 | 195.0 | 2.5 | Aliphatic |
| 189 | 826 | LVALAAPIIAVP | 12 | 41.3 | 211.7 | 2.4 | Aliphatic |
| 190 | 827 | IAAVLAAPALVP | 12 | 57.3 | 187.5 | 2.2 | Aliphatic |
| 191 | 828 | IALLAAPIIAVP | 12 | 41.3 | 220.0 | 2.4 | Aliphatic |
| 192 | 829 | AALALVAPVIVP | 12 | 50.2 | 203.3 | 2.4 | Aliphatic |
| 193 | 830 | IALVAAPVALVP | 12 | 57.3 | 203.3 | 2.4 | Aliphatic |
| 194 | 831 | IIVAVAPAAIVP | 12 | 43.2 | 203.3 | 2.5 | Aliphatic |
| 195 | 832 | AVAAIVPVIVAP | 12 | 43.2 | 195.0 | 2.5 | Aliphatic |
| 196 | 843 | AVLVLVAPAAAP | 12 | 41.3 | 219.2 | 2.5 | Aliphatic |
| 197 | 844 | VVALLAPLIAAP | 12 | 41.3 | 211.8 | 2.4 | Aliphatic |
| 198 | 845 | AAVVIAPLLAVP | 12 | 41.3 | 203.3 | 2.4 | Aliphatic |
| 199 | 846 | IAVAVAAPLLVP | 12 | 41.3 | 203.3 | 2.4 | Aliphatic |
| 200 | 847 | LVAIVVLPAVAP | 12 | 50.2 | 219.2 | 2.6 | Aliphatic |
| 201 | 848 | AVAIVVLPAVAP | 12 | 50.2 | 195.0 | 2.4 | Aliphatic |
| 202 | 849 | AVILLAPLIAAP | 12 | 57.3 | 220.0 | 2.4 | Aliphatic |
| 203 | 850 | LVIALAAPVALP | 12 | 57.3 | 211.7 | 2.4 | Aliphatic |
| 204 | 851 | VLAVVLPAVALP | 12 | 57.3 | 219.2 | 2.5 | Aliphatic |
| 205 | 852 | VLAVAAPAVLLP | 12 | 57.3 | 203.3 | 2.3 | Aliphatic |
| 206 | 863 | AAVVLLPIIAAP | 12 | 41.3 | 211.7 | 2.4 | Aliphatic |
| 207 | 864 | ALLVIAPAIAVP | 12 | 57.3 | 211.7 | 2.4 | Aliphatic |
| 208 | 865 | AVLVIAVPAIAP | 12 | 57.3 | 203.3 | 2.5 | Aliphatic |
| 209 | 867 | ALLVVIAPLAAP | 12 | 41.3 | 211.7 | 2.4 | Aliphatic |
| 210 | 868 | VLVAAILPAAIP | 12 | 54.9 | 211.7 | 2.4 | Aliphatic |
| 211 | 870 | VLVAAVLPIAAP | 12 | 41.3 | 203.3 | 2.4 | Aliphatic |
| 212 | 872 | VLAAAVLPLVVP | 12 | 41.3 | 219.2 | 2.5 | Aliphatic |
| 213 | 875 | AIAIVVPAVAVP | 12 | 50.2 | 195.0 | 2.4 | Aliphatic |
| 214 | 877 | VAIIAVPAVVAP | 12 | 57.3 | 195.0 | 2.4 | Aliphatic |
| 215 | 878 | IVALVAPAAVVP | 12 | 50.2 | 195.0 | 2.4 | Aliphatic |
| 216 | 879 | AAIVLLPAVVVP | 12 | 50.2 | 219.1 | 2.5 | Aliphatic |
| 217 | 881 | AALIVVPAVAVP | 12 | 50.2 | 195.0 | 2.4 | Aliphatic |
| 218 | 882 | AIALVVPAVAVP | 12 | 57.3 | 195.0 | 2.4 | Aliphatic |
| 219 | 883 | LAIVPAAIAALP | 12 | 50.2 | 195.8 | 2.2 | Aliphatic |
| 220 | 885 | LVAIAPAVAVLP | 12 | 57.3 | 203.3 | 2.4 | Aliphatic |
| 221 | 887 | VLAVAPAVAVLP | 12 | 57.3 | 195.0 | 2.4 | Aliphatic |
| 222 | 888 | ILAVVAIPAAAP | 12 | 54.9 | 187.5 | 2.3 | Aliphatic |
| 223 | 889 | ILVAAAPIAALP | 12 | 57.3 | 195.8 | 2.2 | Aliphatic |
| 224 | 891 | ILAVAAIPAALP | 12 | 54.9 | 195.8 | 2.2 | Aliphatic |
| 225 | 893 | VIAIPAILAAAP | 12 | 54.9 | 195.8 | 2.3 | Aliphatic |
| 226 | 895 | AIIIVVPAIAAP | 12 | 50.2 | 211.7 | 2.5 | Aliphatic |
| 227 | 896 | AILIVVAPIAAP | 12 | 50.2 | 211.7 | 2.5 | Aliphatic |
| 228 | 897 | AVIVPVAIIAAP | 12 | 50.2 | 203.3 | 2.5 | Aliphatic |
| 229 | 899 | AVVIALPAVVAP | 12 | 57.3 | 195.0 | 2.4 | Aliphatic |
| 230 | 900 | ALVAVIAPVVAP | 12 | 57.3 | 195.0 | 2.4 | Aliphatic |

TABLE 2-6
[Newly Developed Hydrophobic CPPs - 240 aMTDs That All Critical Factors Are
Considered and Satisfied (Sequence ID No 231-240)]

| | | | | Rigidity/ | Sturctural | | |
|---|---|---|---|---|---|---|---|
| Sequence | | | | Flexibility | Feature | Hydropathy | Residue |
| ID Number | aMTD | Sequences | Length | (II) | (Al) | (GRAVY) | Structure |
| 231 | 901 | ALVAVLPAVAVP | 12 | 57.3 | 195.0 | 2.4 | Aliphatic |
| 232 | 902 | ALVAPLLAVAVP | 12 | 41.3 | 203.3 | 2.3 | Aliphatic |
| 233 | 904 | AVLAVVAPVVAP | 12 | 57.3 | 186.7 | 2.4 | Aliphatic |
| 234 | 905 | AVIAVAPLVVAP | 12 | 41.3 | 195.0 | 2.4 | Aliphatic |
| 235 | 906 | AVIALAPVVVAP | 12 | 57.3 | 195.0 | 2.4 | Aliphatic |
| 236 | 907 | VAIALAPVVVAP | 12 | 57.3 | 195.0 | 2.4 | Aliphatic |
| 237 | 908 | VALALAPVVVAP | 12 | 57.3 | 195.0 | 2.3 | Aliphatic |
| 238 | 910 | VAALLPAVVVAP | 12 | 57.3 | 195.0 | 2.3 | Aliphatic |
| 239 | 911 | VALALPAVVVAP | 12 | 57.3 | 195.0 | 2.3 | Aliphatic |
| 240 | 912 | VALLAPAVVVAP | 12 | 57.3 | 195.0 | 2.3 | Aliphatic |
| | | | | 52.6 ± 5.1 | 201.7 ± 7.8 | 2.3 ± 0.1 | |

TABLE 3
[Summarized Critical Factors of aMTD after
In-Depth Analysis of Experimental Results]
Summarized Critical Factors of aMTD

| | Newly Designed CPPs |
|---|---|
| Critical Factor | Range |
| Bending Potential | Proline presences in the |
| (Proline Position: PP) | middle (5′, 6′, 7′ or 8′) and |
| | at the end (12′) of peptides |
| Rigidity/Flexibility | 41.3-57.3 |
| (Instability Index: II) | |
| Structural Feature | 187.5-220.0 |
| (Aliphatic Index: AI) | |
| Hydropathy | 2.1-2.6 |
| (Grand Average of | |
| Hydropathy) | |
| Length | 12 |
| (Number of Amino Acid) | |
| Amino acid Composition | A, V, I, L, P |
| Secondary Structure | α-Helix is favored |
| | but not required |

These examined critical factors are within the range that we have set for our critical factors; therefore, we were able to confirm that the aMTDs that satisfy these critical factors have much higher cell-permeability and intracellular delivery potential compared to reference hydrophobic CPPs reported during the past two decades.

2. Development of TFF1 Recombinant Proteins Fused to aMTD and Solubilization Domain

High solubility, yield, stability, and cell-/tissue-permeability are essentially required together with their functional activity (anti-cancer effect) to determine whether they are eligible to have therapeutic applicability in clinic. Therefore, additional modifications were strongly recommended on the conventional recombinant TFF1 proteins fused to previously developed MTD.

2-1. The Advanced Hydrophobic CPP—aMTD43 and aMTD165

Based on these analytical data of hydrophobic CPPs published, 240 advanced MTDs (aMTDs) sequences have been designed and developed based on 6 critical factors (TABLES 2-1 to 2-6 and 3). Based on these six critical factors proven by experimental data, newly designed aMTD have been developed for their practical therapeutic usage to facilitate protein translocation across the plasma membrane.

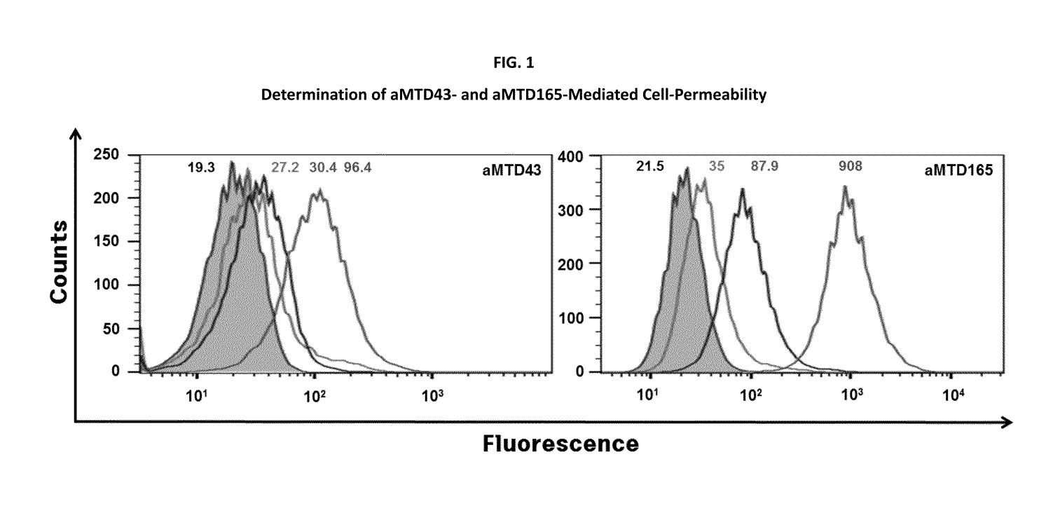

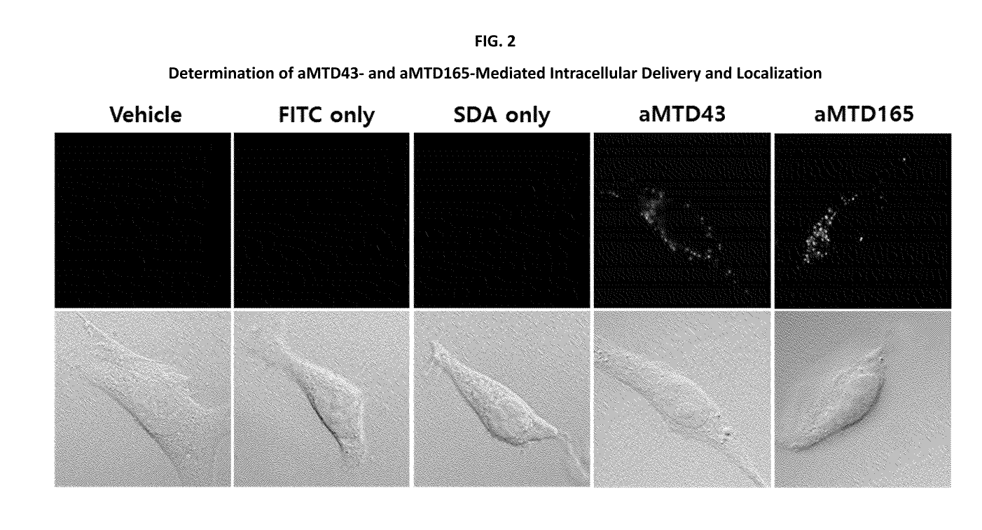

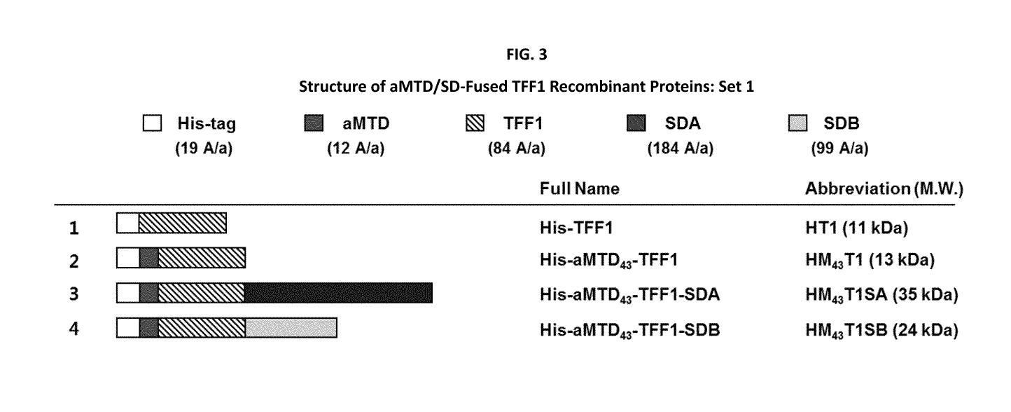

For this present invention, aMTD/SD-fused TFF1 recombinant proteins have been developed by adopting aMTD43 and aMTD165 that satisfied all 6 critical factors. We found that aMTD43 and aMTD165 had a potential to enhance the uptake of a His-tagged enhanced green fluorescent protein (EGFP) in RAW264.7 cells as assessed by flow cytometry. Both peptides promoted greater cellular uptake of an EGFP cargo protein by cultured NIH3T3 cells than SDA only (FIGS. 1 and 2).

### 2-2. Selection of Solubilization Domain for Recombinant Proteins

In addition with aMTD, we adopted non-functional protein domain (solubilization domain: SD; TABLE 5) to improve solubility, yield, and stability of the recombinant proteins. In previous study, to develop recombinant aMTD/SD-fused TFF1 recombinant proteins as protein-based biotherapeutics to treat gastric cancer, recombinant cargo (TFF1) proteins fused to conventional hydrophobic CPPs—MTDs could be expressed in bacteria system, purified with single-step affinity chromatography, but protein dissolved in physiological buffers (e.q. PBS, DMEM or RPMI1640 etc.) was highly insoluble and had low yield as a soluble form. This problem is extremely crucial in terms of the fact whether these proteins could go ahead for further pre-clinical and clinical development.

According to this specific aim, 5 solubilization domains were selected and information of these SDs are shown TABLE 5.

TABLE 5
[Characteristics of Solubilization Domains]

| | | | Protein | | Instability | |
|---|---|---|---|---|---|---|
| SD | Genbank ID | Origin | (kDa) | pI | Index (II) | GRAVY |
| A | CP000113.1 | Bacteria | 23 | 4.6 | 48.1 | −0.1 |
| B | BC086945.1 | Pansy | 11 | 4.9 | 43.2 | −0.9 |
| C | CP012127.1 | Human | 12 | 5.8 | 30.7 | −0.1 |
| D | CP012127.1 | Bacteria | 23 | 5.9 | 26.3 | −0.1 |
| E | CP011550.1 | Human | 11 | 5.3 | 44.4 | −0.9 |
| F | NG_034970 | Human | 34 | 7.1 | 56.1 | −0.2 |

### 2-3. Preparation of TFF1 Recombinant Proteins

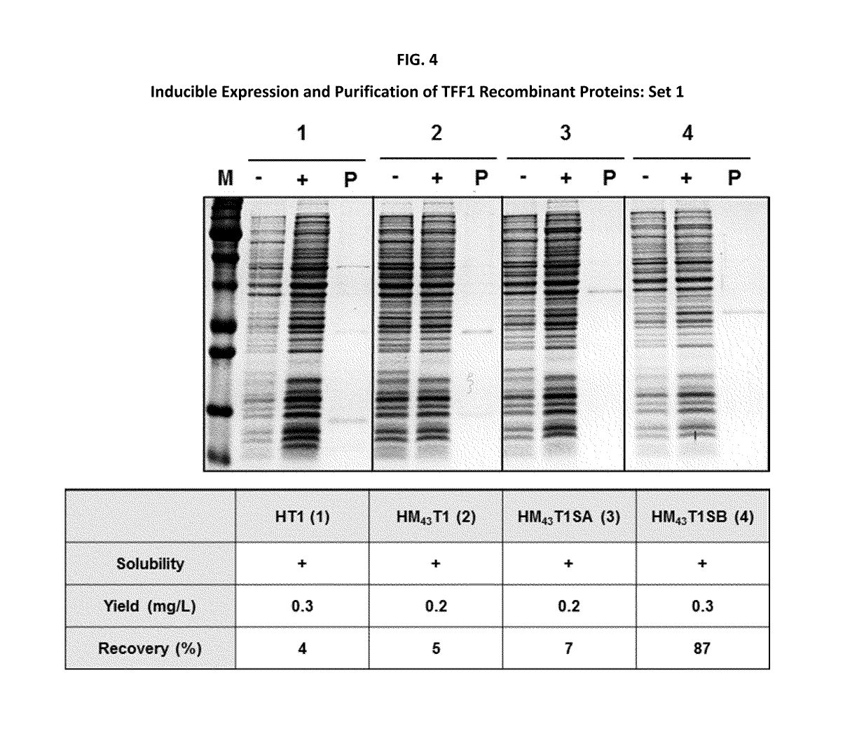

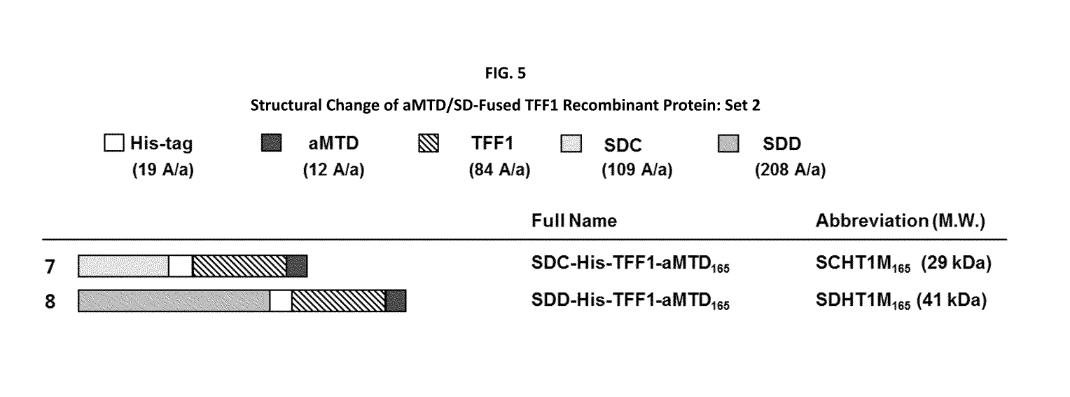

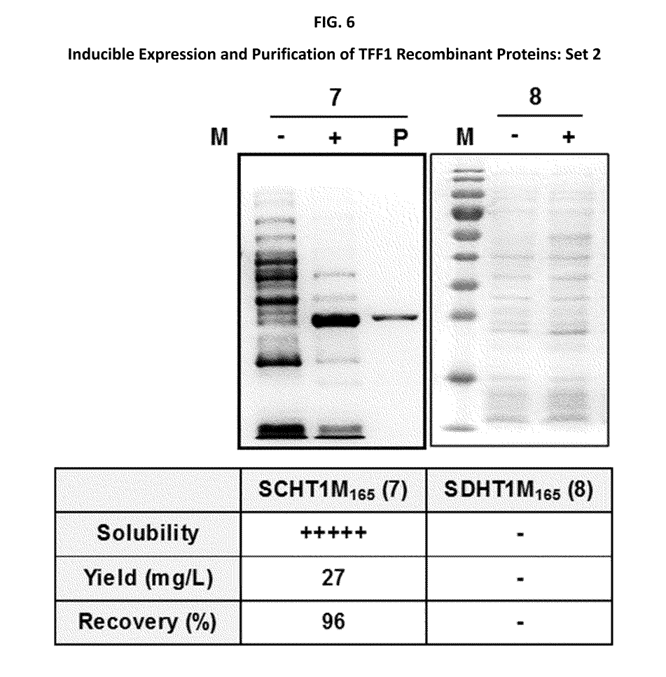

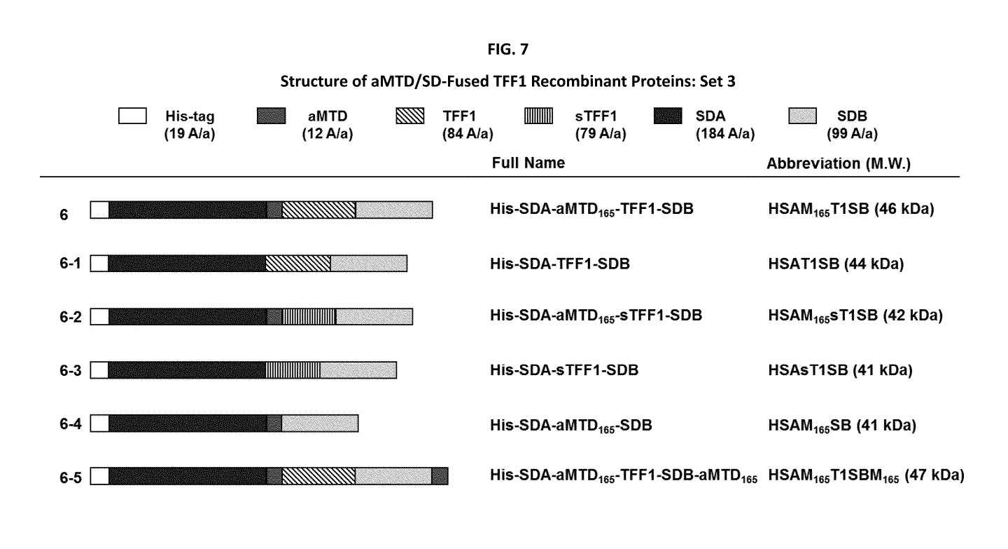

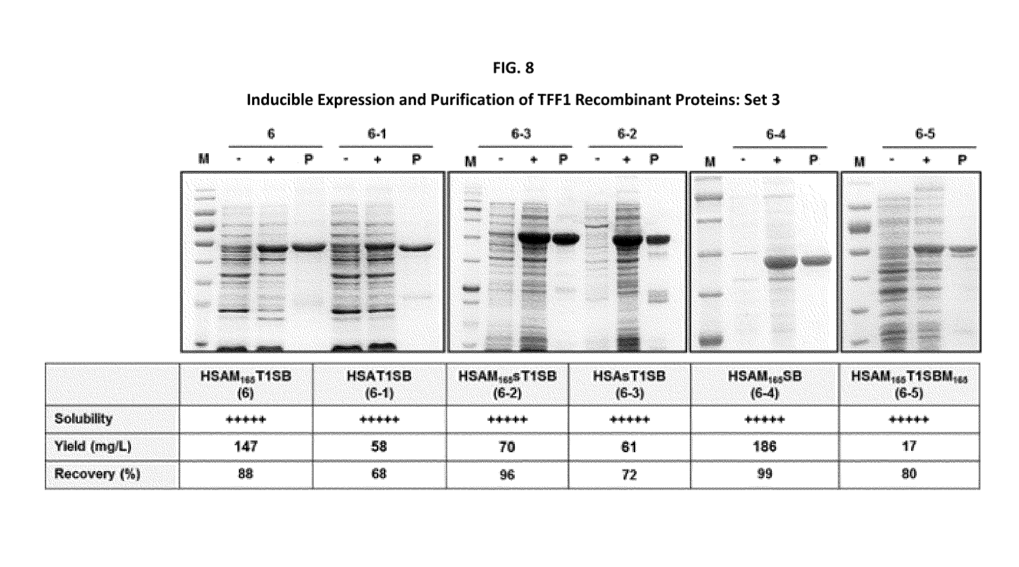

To overcome the limitations of high insolubility and low yield, we developed a newly recombinant TFF1 fused to aMTD and the SDs. To develop stable aMTD/SD-fused TFF1 recombinant proteins with better yield and solubility, total of three sets of TFF1 recombinant protein clones were designed and developed. In Set 1, TFF1 recombinant protein clones fused with aMTD43 and either SDA or SDB fused to C-terminus of the protein were developed. Recombinant proteins in Set 1 have shown relatively weak expression with very low solubility and yield (FIG. 4). To develop more stable aMTD/SD-fused TFF1 recombinant proteins, we performed the cloning for aMTD/SD-fused TFF1 recombinant proteins containing aMTD165 and SDC or SDD (FIG. 5). Despite the fact that Set 2 has shown higher yield and solubility compared to set 1, we were not satisfied with the protein solubility and yield in Set 2 of aMTD/SD-fused TFF1 recombinant proteins (FIG. 6). Therefore, Set 3 recombinant protein clones have been designed and developed for more stable, improved solubility and yielding TFF1 recombinant protein. In Set 3, TFF1 recombinant proteins were developed by replacing the aMTD and SDs to the combination of aMTD165 and both SDA and SDB on each ends of the protein (FIG. 7). TFF1 protein has a signal sequence at the N terminal. From analyzing this signal sequence based on our critical factors, we determined that this signal sequence has a cell-permeable potential (TABLE 6). Therefore, we additionally developed TFF1 (sTFF1) by removing these 15 amino acid signal sequences. These recombinant protein clones were expressed in E. coli BL21-Codon plus (DE3). The recombinant proteins were purified under the naturing conditions and the yields of soluble protein 45 mg/L (FIG. 8). Set 3 aMTD/SD-TFF1 recombinant protein has been significantly enhanced (250 fold) in solubility and yield. Therefore, aMTD/SD-fused TFF1 recombinant protein 6 in Set 3, with highest solubility/yield, has been selected to determine the cell-/tissue-permeability in vitro and xenograft model experiments.

TABLE 6
[Characteristics of TFF1 signal sequence]

| | | | Rigidity/ | Structural | Hydrop- |
|---|---|---|---|---|---|
| | | Length | Flexibility | Feature | athy |
| Group | Sequences | (a.a) | (II) | (AI) | (GRAVY) |
| Signal | VICALVLVSMLAL | 13 | 9.23 | 232.31 | 3.04 |
| sequence | | | | | |

PCR primers for the His-tagged solubilization domain recombinant proteins fused to aMTD43 and aMTD165 are summarized in TABLE 7. cDNA and amino acid sequences of histidine tag are provided in SEQ ID NO: 1 and 2, and cDNA and amino acid sequences of aMTDs and the peptides are indicated in SEQ ID NO: 3 and 4, respectively. cDNA and amino acid sequences are displayed in SEQ ID NO: 5 and 6 (TFF1), SEQ ID NO: 7 and 8 (SDA), SEQ ID NO: 9 and 10 (SDB), SEQ ID NO: 11 and 12 (SDC), SEQ ID NO: 13 and 14 (SDD), SEQ ID NO: 15 and 16 (SDE) and SEQ ID NO: 17 and 18 (SDF), respectively.

TABLE 7
[PCR Primers for His-tagged TFF1 Proteins Fused to aMTD]

| | | Recombinant | | |
|---|---|---|---|---|
| Cargo | aMTD | Protein | 5′ Primers | 3′ Primers |
| TFF1 | Non | HT1 | C1/F: | C1/R: |
| | | | 5′-CCACCATGGAGAACAAGGT | 5′-TTTGAATTCTCAGTAGGGC |
| | | | GATCTGC-3′ | CGCCACACGGCCT-3′ |
| | aMTD43 | HM₄₃T1 | C2/F: | C2/R: |
| | | | 5′-GGAATTCCATATGCTGCTGG | 5′-CCCGGATCCTAAAAATTCA |
| | | | CGGCGCCGCTGGTGGTGGCGGCG | CACTCCTCTTCTGGAGGGAC-3′ |
| | | | GTGCCGGCCACCATGGAGAACAA | |
| | | | GGTGATCTGC-3′ | |
| | | HM₄₃T1SA | C3/F: | C3/R: |
| | | | 5′-GGAATTCCATATGCTGCTGG | 5′-CCCAAGCTTAAATTCACACT |
| | | | CGGCGCCGCTGGTGGTGGCGGCG | CCTCTTCTGGAGGGAC-3′ |
| | | | GTGCCGGCCACCATGGAGAACAA | |
| | | | GGTGATCTGC-3′ | |
| | | HM₄₃T1SB | C4/F: | C4/R: |
| | | | 5′-GGAATTCCATATGCTGCTGG | 5′-CCCAAGCTTAAATTCACACT |
| | | | CGGCGCCGCTGGTGGTGGCGGCG | CCTCTTCTGGAGGGAC-3′ |
| | | | GTGCCGGCCACCATGGAGAACAA | |
| | | | GGTGATCTGC-3′ | |
| | aMTD165 | HSAM₁₆₅T1SB | C6/F: | C6/R: |
| | | | 5′-CGCGGATCCGCGCTGGCGG | 5′-GGGTTTGTCGACAAATTCAC |
| | | | TGCCGGTGGCGCTGGCGATTGTG | ACTCCTCTTC-3′ |
| | | | CCGATGGCCACCATGGAGAACAA | |
| | | | G-3′ | |
| | | HSCT1M₁₆₅ | C7/F: | C7/R: |
| | | | 5′-CGCGGATCCATGGCCACCA | 5′-CCCAAGCTTTTACGGCACAA |
| | | | TGGAGAAC-3′ | TCGCCAGCGCCACCGGCACCGCC |
| | | | | AGCGCAAATTCACACTCCTCTTC- |
| | | | | 3′ |
| | | HSDT1M165 | C8/F: | C8/R: |
| | | | 5′-CGCGGATCCATGGCCACCAT | 5′-CCCAAGCTTTTACGGCACAA |
| | | | GGAGAAC-3′ | TCGCCAGCGCCACCGGCACCGCC |
| | | | | AGCGCAAATTCACACTCCTCTTC- |
| | | | | 3′ |
| | | HSAT1SB | C6-1/F: | C6-1/6-2/6-3/6-5R: |
| | | | 5′-CGCGGATCCGCGCTGGCGG | 5′-GGGTTTGTCGACAAATTCAC |
| | | | TGCCGGTGGCGCTGGCGATTGTG | ACTCCTCTTC-3′ |
| | | | CCGATGGCCACCATGGAGAACAA | |
| | | | G-3′ | |
| | | HSAM165sT1SB | C6-2/F: | |
| | | | 5′-CGCGGATCCGCGCTGGCGGT | |
| | | | GCCGGTGGCGCTGGCGATTGTGC | |
| | | | CGGGCACCCTGGCCGAGGCCCAG | |
| | | | ACAGAG-3′ | |
| | | HSAMsT1SB | C6-3/F: | |
| | | | 5′-CGCGGATCCGGCACCCTGGCC | |
| | | | GAGGCCCAGACAGAG-3′ | |
| | | HSAM₁₆₅T1SBM₁₆₅ | C6-5/F: | |
| | | | 5′-CGCGGATCCATGGCCACCA | |
| | | | TGGAGAACAAGGTG-3′ | |

3. aMTD/SDs-Fused TFF1 Recombinant Proteins Significantly Increase Cell- and Tissue-Permeability
3-1. aMTD/SDs-Fused TFF1 Recombinant Proteins are Cell-Permeable

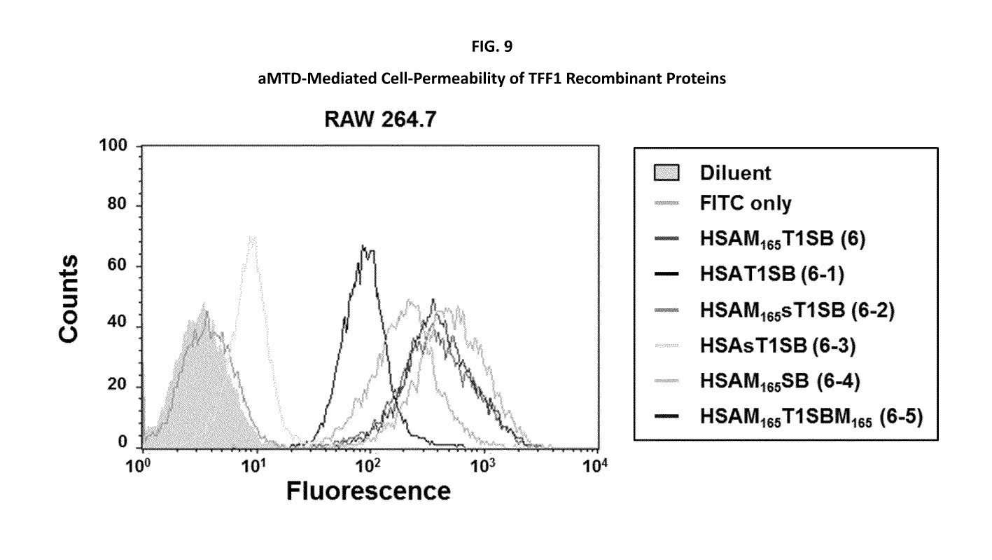

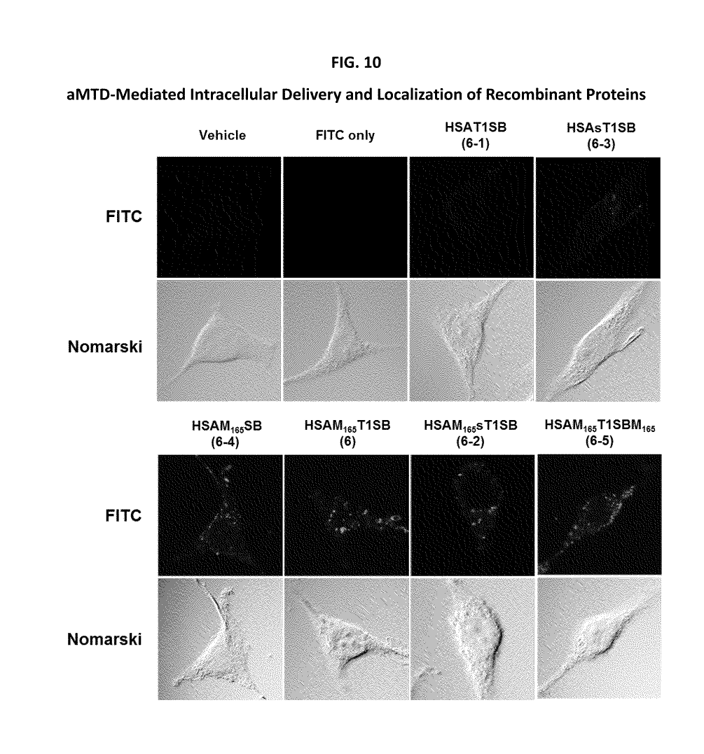

Cell permeability of TFF1 recombinant protein was evaluated in RAW 264.7 cells after 1 hour of protein treatment. aMTD/SD-fused TFF1 recombinant proteins were conjugated to FITC, according to the manufacturer's instructions. Protein uptake of TFF1 proteins containing aMTD165 (HSAM165T1SB, HSAM165sT1SB, HSAM165T1SB M165) in RAW264.7 cell (FIG. 9).

In contrast, FITC-labeled TFF1 lacking aMTD (HSAT1SB, HSAsT1SB) was not detectable in RAW 264.7 cell. Similar Results were observed in NIH3T3 cells, using fluorescence confocal laser scanning microscopy to determine intracellular localization (FIGURE ID NO. 10). TFF1 proteins containing aMTD165 (HSAM₁₆₅T1SB, HSAM₁₆₅sT1SB, HSAM₁₆₅T1SB M₁₆₅) and efficiently entered the cells and were localized to various extents in the cytoplasm. In contrast, TFF1 protein (HSAT1SB, HSAsT1SB), containing only 6×His and the SDs, did not appear to enter the cells.

3-2. aMTD/SDs-Fused TFF1 Recombinant Proteins Enhance the Systemic Delivery to a Variety of Tissues

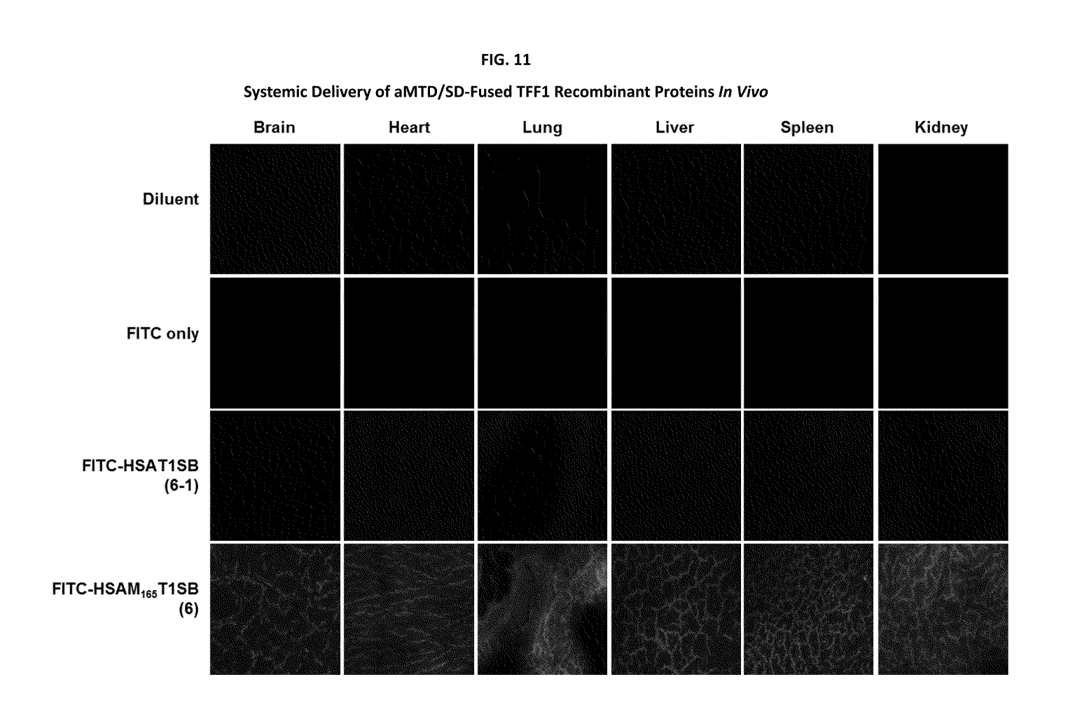

Next, to further investigate in vivo delivery of TFF1 recombinant proteins, FITC-labeled TFF1 proteins were monitored following intraperitoneal (IP) injections in mice. Tissue distributions of fluorescence-labeled-TFF1 proteins in different organs were analyzed by fluorescence microscopy. As shown in FIG. 11, aMTD165 enhanced the systemic delivery of TFF1 protein to variety of tissues including brain, heart, lung, liver, spleen and kidney. However, the recombinant TFF1 lacking aMTD (HSAT1SB) showed limited tissue-permeability in various organs. Therefore, the aMTD165/SD-fused TFF1 recombinant proteins have significantly higher cell- and tissue-permeability as compared to the recombinant protein lacking aMTD. Thus, we determined aMTD165/SD-fused TFF1 recombinant protein which shows cell-/tissue-permeability as cell-permeable TFF1 (CP-TFF1).

4. Membrane Integrity and Fluidity were Essential for aMTD165-Delivery Mechanism

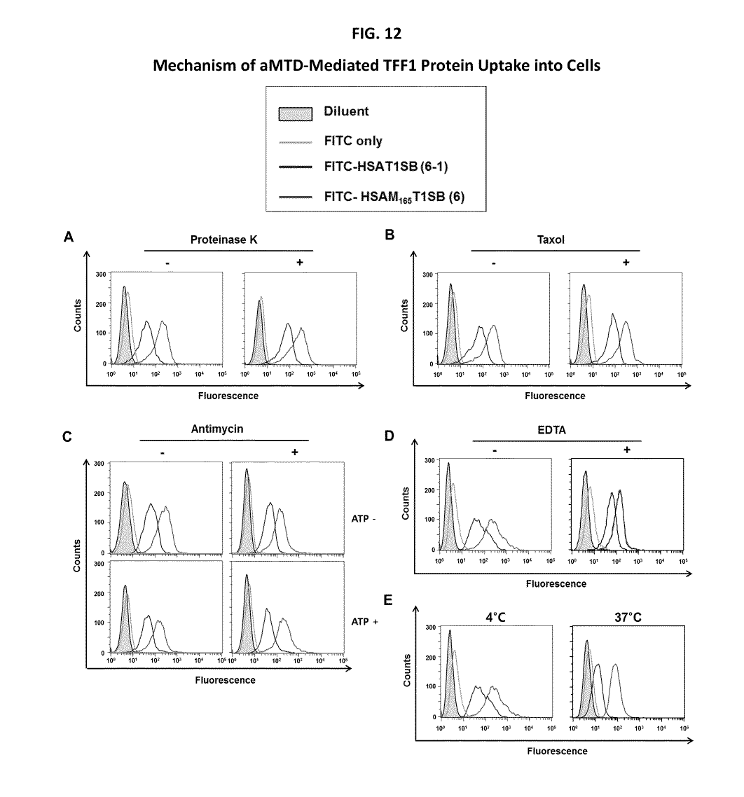

Since the aMTD165 outperformed over other transduction domains tested, we next investigated the mechanism of aMTD165-mediated protein uptake. The hydrophobic aMTD is thought to enter the cells directly by penetrating the plasma membrane. Several lines of evidence suggested that endocytosis was not the major route of entry by aMTD165-fused TFF1 proteins. In particular, uptake was unaffected by the treatment of cells with proteases, microtubule inhibitors or the ATP-depleting agent, antimycin. Conversely, HSAM165T1SB uptake was blocked by the conditions affecting membrane fluidity (temperature) and integrity (EDTA) (FIG. 12).

5. CP-TFF1 Enhances the Penetration into Gastric Cancer Cells and Systemic Delivery to Stomach

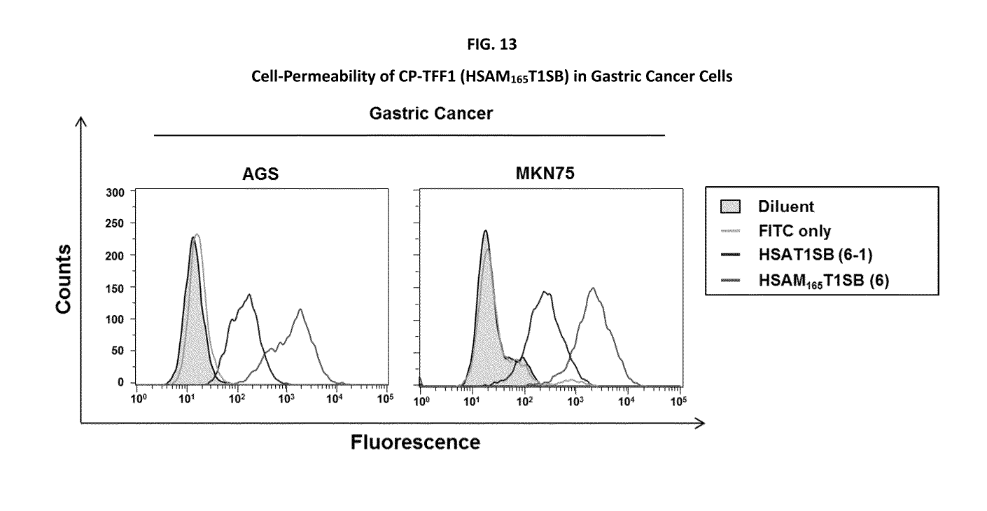

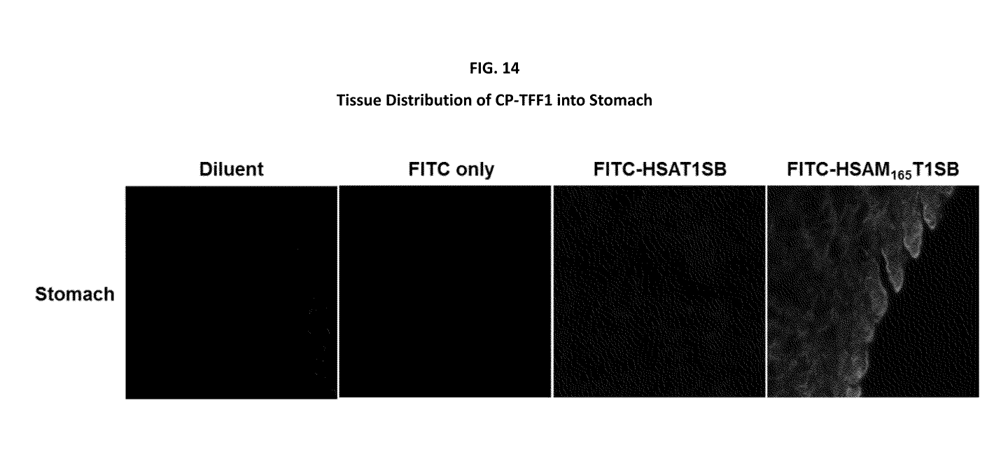

To determine the cell-permeability of CP-TFF1 in the gastric cancer cells, cellular uptake of FITC-labeled TFF1 recombinant proteins was quantitatively evaluated by flow cytometry. FITC-HSAM165T1SB recombinant protein (CP-TFF1) promoted the transduction into cultured AGS and MKN75 gastric cancer cells (FIG. 13). In addition, CP-TFF1 enhanced the systemic delivery to stomach after intraperitoneal injection (FIG. 14). Therefore, these data indicate that CP-TFF1 could be intracellularly delivered and distributed to the target cells and stomach tissue, contributing for beneficial biotherapeutic effects.

### 6. CP-TFF1 Suppressed Proliferation of Gastric Cancer Cells

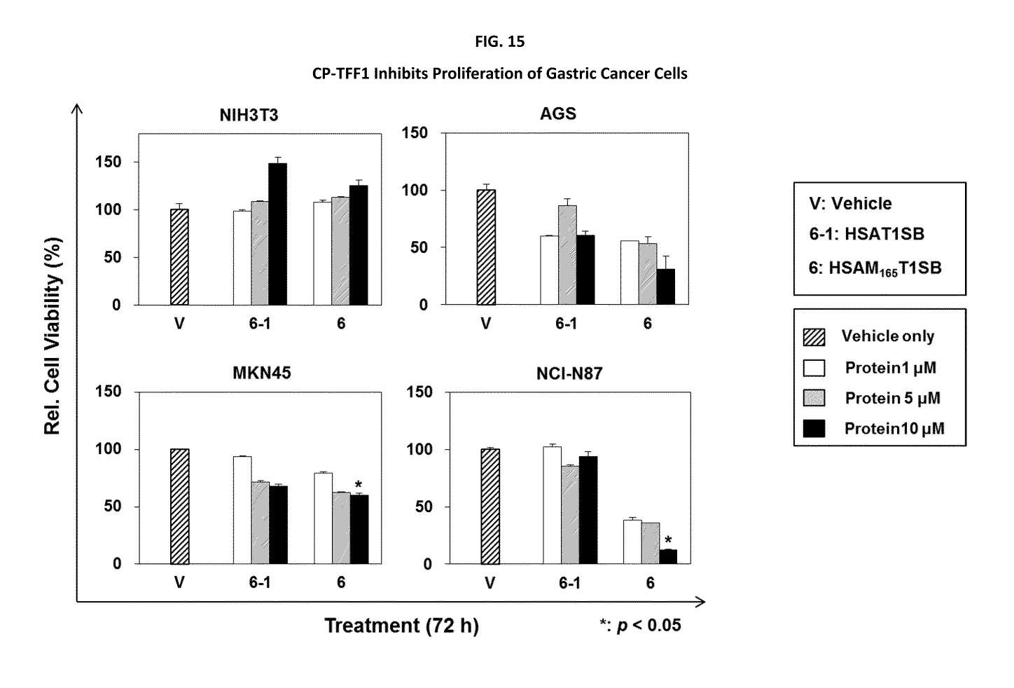

To examine the effect of CP-TFF1 on cancer cell proliferation, we performed the CellTiter-Glo Luminescent Cell Viability Assay. As shown in FIG. 15, CP-TFF1 (HSAM165T1SB) was the most potent inducer of cytotoxicity—over 60%˜80% in 10 μM treatment (p<0.05) than other proteins including HSAT1SB or vehicle alone (i.e. exposure of cells to culture media without recombinant proteins) in AGS and NCI-N87 cells. Also, CP-TFF1 mildly suppressed the proliferation in other gastric cancer cells (MKN45). However, CP-TFF1 proteins neither appeared to induce cytotoxicity in NIH3T3 cells nor were cell viability affected, even after exposing these cells to equal concentrations (10 μM) of protein over 3 days. These results suggest that the protein is merely non-toxic to cells and indicates that CP-TFF1 has a great ability to inhibit cell survival-associated phenotypes in gastric cancer cells.

### 7. CP-TFF1 Protein Inhibited Migration of Gastric Cancer Cells

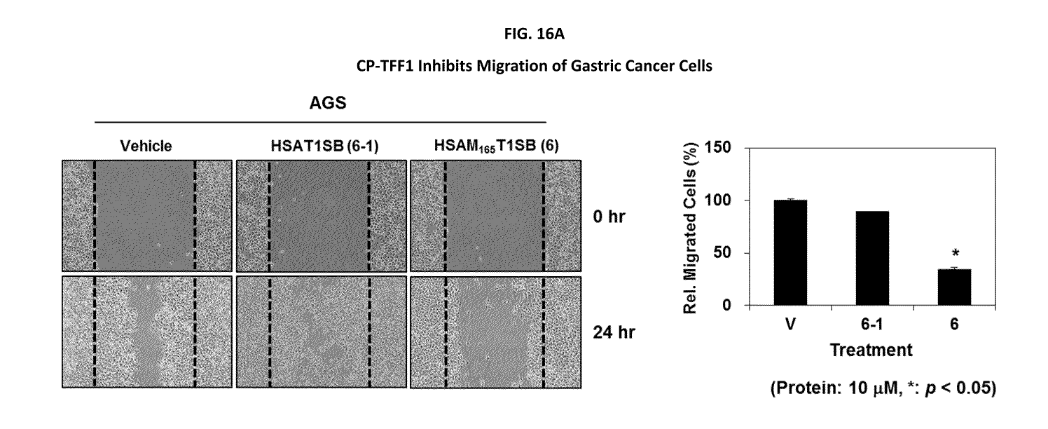

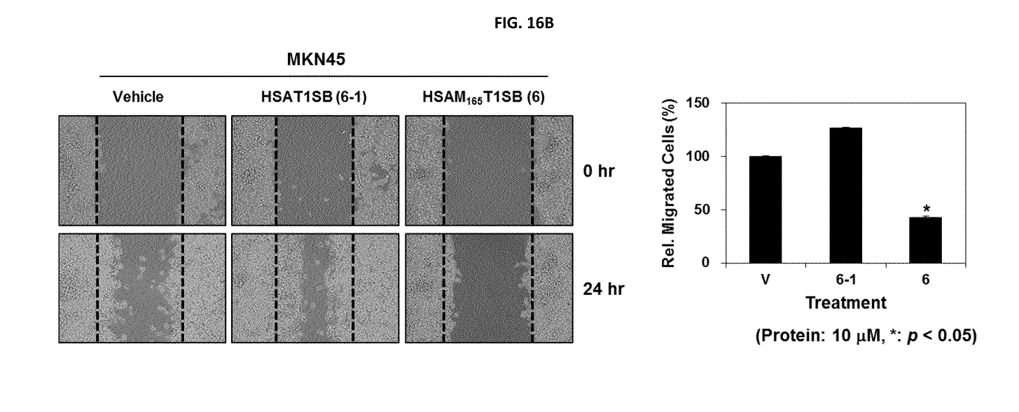

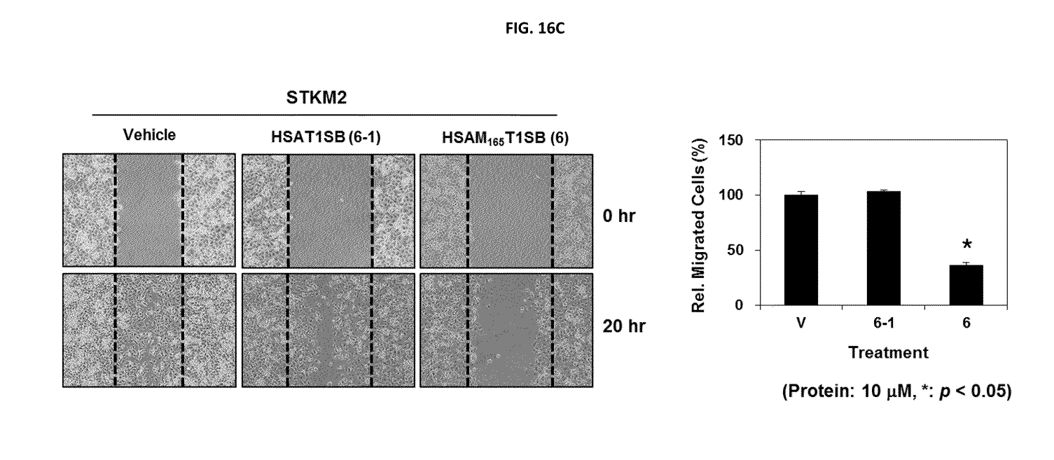

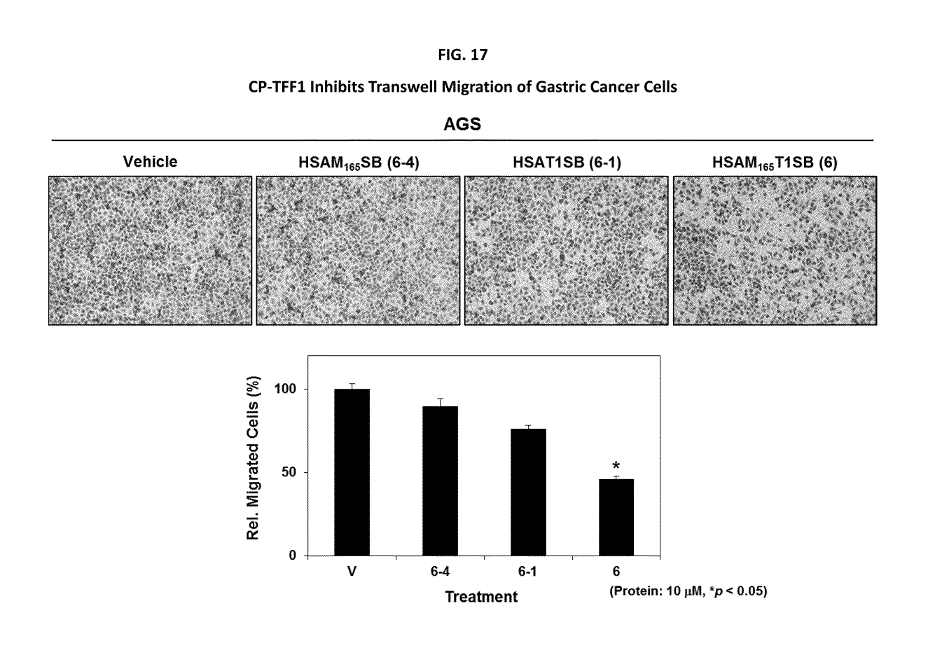

We next examined the wound healing assay and Transwell assay to assess the effects of CP-TFF1 proteins on gastric cancer cell migration (AGS, MKN45 and STKM2 cells). The wounds were produced by scraping of the cell monolayer with sterile white tip. CP-TFF1 protein (HSAM165T1SB) suppressed repopulation of the wounded monolayer (FIGS. 16A to 16C). Consistent with this, AGS cells treated with CP-TFF1 (HSAM165T1SB) also showed significant inhibitory effect on their Transwell migration compared with untreated cells (Vehicle) (FIG. 17). However, TFF1 protein lacking aMTD165 (HST1SB) was not affected on gastric cancer cell migration. Taken together, these data indicate that CP-TFF1 contributes to inhibit tumorigenic activities of gastric cancer cells.

### 8. CP-TFF1 Protein Induced Apoptosis in Gastric Cancer Cells

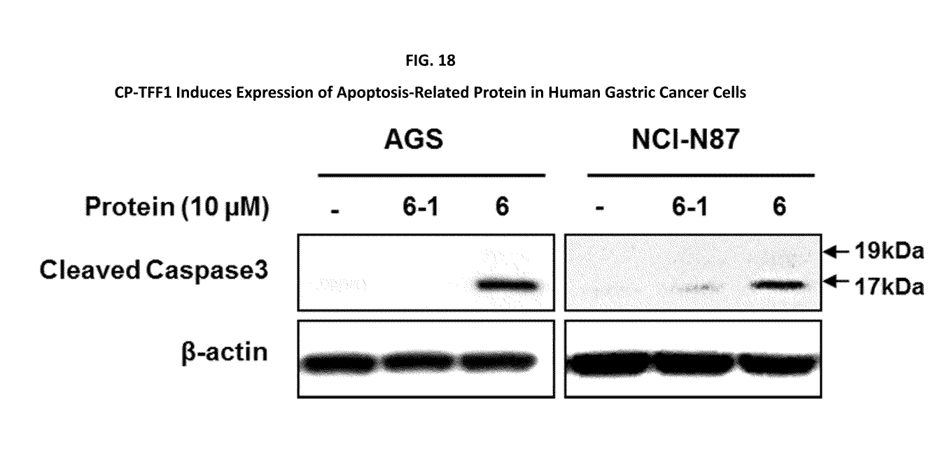

To further examine the underlying mechanisms of the anti-cancer effect of CP-TFF1, we performed western blot analysis. Human gastric cancer cells (AGS, NCI-N87) were treated with 10 μM of CP-TFF1 proteins (HSAM165T1SB) for 24 hr. Compared to control recombinant protein (HST1SB)-treated cells, cells treated with CP-TFF1 showed significantly increased cleaved Caspase-3 which plays an important role in apoptosis (FIG. 18). Therefore, these data indicate that CP-TFF1 recombinant protein induce the apoptosis in gastric cancer cells.

### 9. CP-TFF1 Suppresses Pro-Tumorigenic Functions in Gastric Cancer Cells

### 9-1. CP-TFF1 Suppressed Tumor Growth in Tumor Block Implanted Xenograft

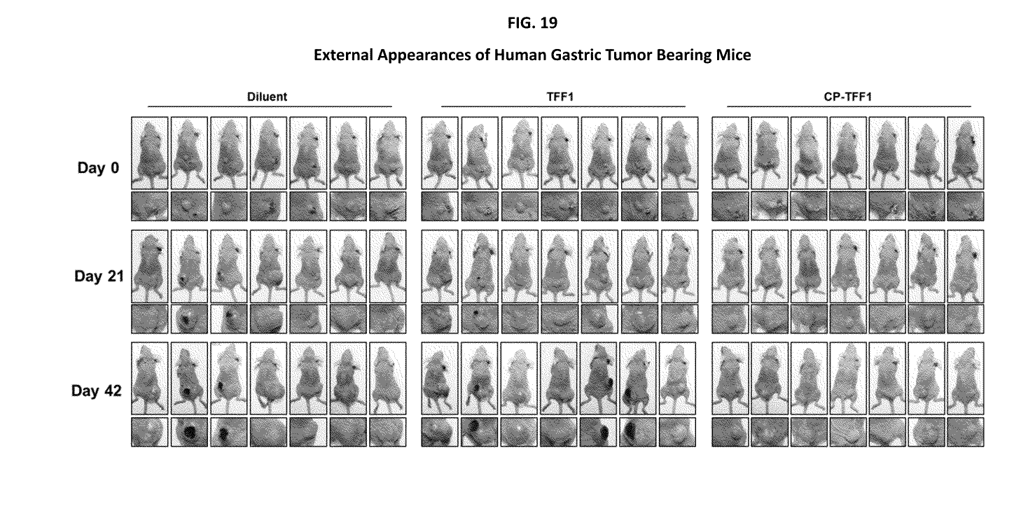

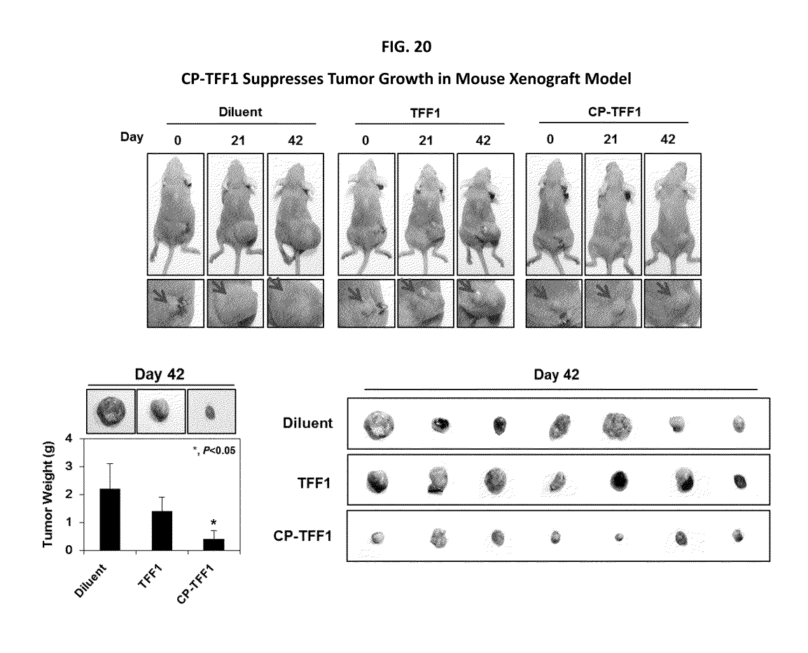

Next, we assessed the anti-tumor activity of CP-TFF1 against human cancer xenografts. Balb/c nu/nu mice were subcutaneously implanted with MKN45 tumor block (2 mm3) into the left back side of the mouse. Then, the mice were intravenously injected with 800 Ng/head recombinant TFF1 proteins (HSAT1SB or HSAM165T1SB) or diluent (DMEM) every day for 3 weeks. Mice were observed for an additional 3 weeks after the treatments ended. It shows phenotypic appearance of mouse treated with diluent, TFF1, CP-TFF1 at Day 0, 21, 42 (each group tested 7 mice) (FIG. 19). Tumor weight was significantly reduced in mice treated with CP-TFF1 (FIG. 20 lower left panel) and the diagram shows the isolated tumor of mice from each group at Day 42 (FIG. 20 lower right panel).

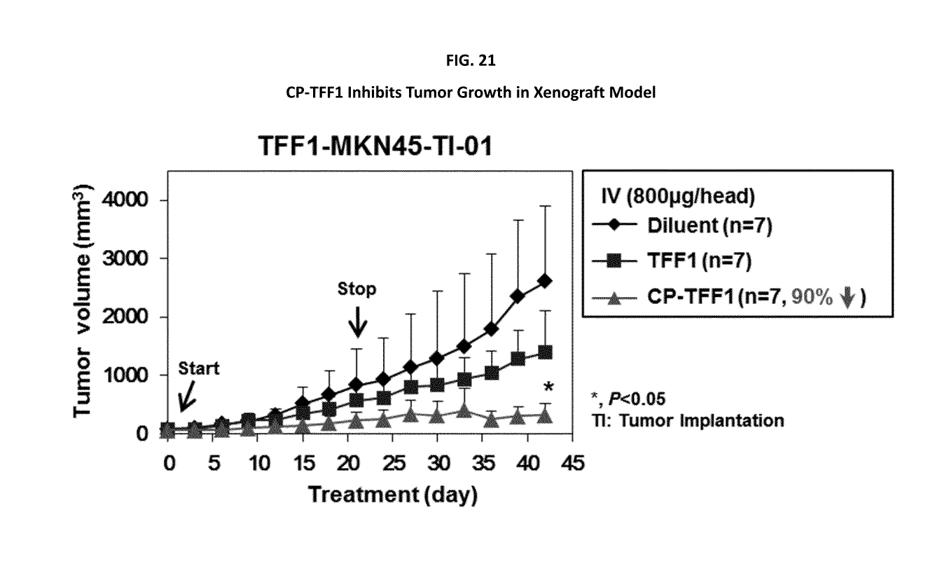

CP-TFF1 (HSAM165T1SB) protein significantly suppressed the tumor growth (p<0.05) during the treatment phase and persisted for at least 3 weeks after the treatment terminated (90% inhibition at day 42) (FIG. 21). While tumor growth was also reduced in mice treated with the HSAT1SB control protein, which lacked aMTD sequences, the effect was not significant. These results suggest that CP-TFF1 inhibits the persistence of established tumors as well as the tumor growth of cancer cells.

### 9-2. CP-TFF1 Regulates the Expression of Tumor-Associated Proteins in Human Tumor Xenograft

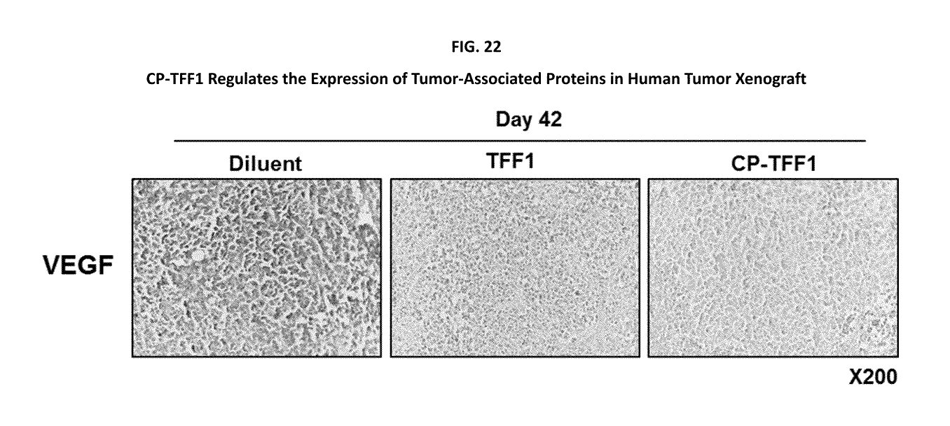

The anti-tumor activity of CP-TFF1 (HSAM165T1SB) at day 42 was accompanied by changes in the expression of biomarkers (FIG. 22). The expressions of vascular endothelial growth factor (VEGF), a pro-angiogenic factor, were inhibited in HSAM165T1SB)-treated tumors. While the expression of VEGF was also reduced in mice treated with the HSAT1SB control protein, which lacked aMTD sequences, the effect was not significant. These in vivo results suggest that CP-TFF1 targets tumor cells directly and may be developed for use as novel therapy against gastric cancer.

### Examples

The following examples are presented to aid practitioners of the invention, to provide experimental support for the invention, and to provide model protocols. In no way are these examples to be understood to limit the invention.

### Example 1

### Construction of Expression Vectors for TFF1 Recombinant Proteins Fused to aMTDs

The expression vectors were designed for TFF1 recombinant protein fused with either one or both SDs (SDA, SDB, SDC, and SDD) and aMTD43 or aMTD165. To acquire expression vectors for TFF1 recombinant proteins, polymerase chain reaction (PCR) had been devised to amplify each designed aMTD43 or aMTD165 fused with TFF1.

The PCR reactions (100 ng genomic DNA, 10 pmol each primer, each 0.2 mM dNTP mixture, 1× reaction buffer and 2.5 U Pfu(+) DNA polymerase (Doctor Protein, Korea)) was digested on the different restriction enzyme site involving 40 cycles of denaturation (95° C.), annealing (58° C.), and extension (72° C.) for 30 seconds each. For the last extension cycle, the PCR reactions remained for 10 minutes at 72° C. TFF1 recombinant protein clones were produced in three sets. Set 1 coding sequence for SDA or SDB fused to C terminus of his-tagged aMTD/SD-TFF1 was cloned at NdeI (5′) and SalI (3′) in pET-28a(+) (Novagen, Madison, Wis., USA) from PCR-amplified DNA segments. PCR primers for the TFF1-fuseded SDA or SDB are summarized in TABLE 8, respectively. Set 2 coding sequence for SDC or SDD fused to N terminus of TFF1 recombinant protein was cloned at BamHI (5′) and HindIII (3′) in pET-32a(+) (Novagen, Madison, Wis., USA) or pET-39b(+) (Novagen, Madison, Wis., USA) from PCR-amplified DNA segments. PCR primers for the TFF1-fused to either SDC or SDD are summarized in TABLES 8 and 9, respectively. Set 3 coding sequence for SDA fused to N terminus and SDB fused to C terminus of aMTD/SD-TFF1 or aMTD/SD-sTFF1 was cloned at NdeI (5′) and XhoI (3′) in pET-28a(+) (Novagen, Madison, Wis., USA) from PCR-amplified DNA segments. DNA ligation was performed using T4 DNA ligase at 4° C. overnight. These plasmids were mixed with competent cells of E. coli DH5-alpha strain on the ice for 30 minutes. These mixture was placed in a water bath at 42° C. for 90 seconds and placed on ice for 2 minutes. Then, the mixture added with LB broth media was recovered in 37° C. shaking incubator for 1 hour. Transformant was plated on LB broth agar plate with kanamycin (50 μg/mL) (Biopure, Johnson, Tenn.) or ampicillin (100 μg/mL) (Biopure, Johnson, Tenn.) before incubating overnight at 37° C. From a single colony, plasmid DNA was extracted; and after the double digestion of Inserts fit restriction enzymes, digested DNA was confirmed at TFF1 (252 bp), sTFF1(207 bp), SDA (297 bp), and SDB (552 bp) by using 1.4% agarose gels electrophoresis.

TABLE 8
[PCR Primers for SD Fused to TFF1 Protein]

| | | Recombinant | | |
|---|---|---|---|---|
| SD | aMTD | Protein | 5′Primers | 3′Primers |
| SDA | aMTD43 | HM₄₃T1SA | C3/F: | C3/R: |
| | | | 5′-CGCGGATCCATGGCAAATATT | 5′-ACGCGTCGACTTACCTCGGCT |
| | | | ACCG-3′ | GCACCGG-3′ |
| | aMTD165 | HSAM₁₆₅T1SB | C6/F: | C6/6-1/6-2/6-3/R: |
| | | HSAT1SB | 5′-GGGTTTCATATGATGGCAAATA | 5′-CGCGGATCCCCTCGGCTGCAC |
| | | HSAM₁₆₅sT1SB | TTACCGTTTTC-3′ | CGGCACGGC-3′ |
| | | HSAMsT1SB | | |
| | | HSAM₁₆₅SB | | C6-4/R: |
| | | | | 5′-ACGCGTCGACCGGCACAATCGC |
| | | | | CAGCGCCACCGGCACCGCCAGCGC |
| | | | | CCTCGGCTGCACCGGCACGGA-3′ |
| | | HSAM₁₆₅T1SBM₁₆₅ | | C6-5/R: |
| | | | | 5′-CGCGGATCCCCTCGGCTGCACC |
| | | | | GGCACGGC-3′ |
| SDB | aMTD43 | HM₄₃T1SB | C4/F: | C3/R: |
| | | | 5′-CGCGGATCCATGGCAGAAC | 5′-ACGCGTCGACTTACCTCG |
| | | | AAAGCG-3′ | GCTGCACCGG-3′ |
| | aMTD165 | HSAM₁₆₅T1SB | C6/F: | C6/6-1/6-2/6-3/6-4/R: |
| | | HSAT1SB | 5′ ACGCGTCGACATGGCAGAAC | 5′-CCGCTCGAGGTTAAAGGGTTT |
| | | HSAM₁₆₅sT1SB | AAAGCGAC-3′ | CCGAAGGCTTG-3′ |
| | | HSAMsT1SB | | |
| | | HSAM₁₆₅SB | | |
| | | HSAM₁₆₅T1SBM₁₆₅ | | C6-5/R: |
| | | | | 5′-CCGCTCGAGGTTACACAATCGC |
| | | | | CAGCGCCACCGGCACCGCCAGCGC |
| | | | | CGGAAGGGTTTCCGAAGGCTTGGC |
| | | | | TATC-3′ |

### Example 2

### Purification and Preparation of aMTD/SD-Fused TFF1 Recombinant Protein

Denatured recombinant proteins were lysed using denature lysis buffer (8 M Urea, 10 mM Tris, 100 mM NaH2PO4) and purified by adding Ni-NTA resin. Resin bound to proteins were washed 3 times with 30 mL of denature washing buffer (8 M Urea, 10 mM Tris, 20 m imidazole, 100 mM NaH2PO4). Proteins were eluted 3 times with 30 mL of denature elution buffer (8 M Urea, 10 mM Tris, 250 mM imidazole). After purification, they was dialyzed twice against a refolding buffer (550 mM Guanidine-HCl, 440 mM L-Arginine, 50 mM Tris, 100 mM NDSB, 150 mM NaCl, 2 mM reduced glutathione and 0.2 mM oxidized glutathione). Finally, they were dialyzed against a physiological buffer such as DMEM at 4° C. until the dialysis was over 300×105 times. Concentration of purified proteins was quantified using Bradford assay according to the manufacturer's instructions. After purification, they were dialyzed against DMEM as indicated above. Finally, SDS-PAGE analysis was conducted to confirm the presence of target protein (FIG. 6).

The His-tagged aMTD/SD-fused TFF1 recombinant proteins (FIG. 8, Set 3) were purified from E. coli BL21-Gold (DE3) competent cells (Agilent Technologies, Santa Clara, USA) grown to an A600 of 0.6 and induced overnight at 25° C. with 0.7 mM/IPTG. The E. coli cultures were harvested by centrifugation at 5,000× rpm for 10 minutes, and the supernatant was discarded. The pellet was resuspended in the lysis buffer (50 mM NaH2PO4, 10 mM Imidazol, 300 mM NaCl, pH 8.0). The cell lysates were sonicated on ice using a sonicator (Sonics and Materials, Inc., Newtowen, Conn.) equipped with a probe. After centrifuging the cell lysates at 5,000× rpm for 10 minutes to pellet the cellular debris, the supernatant was incubated with lysis buffer-equilibrated Ni-NTA resin (Qiagen, Hilden, Germany) gently by open-column system (Bio-rad, Hercules, Calif.). After washing protein-bound resin with 30 ml native wash buffer (50 mM NaH2PO4, 20 mM Imidazol, 300 mM NaCl, pH 8.0), the bounded proteins were eluted with 20 ml elution buffer (50 mM NaH2PO4, 250 mM Imidazol, 300 mM NaCl, pH 8.0). Total concentration of protein was quantified using Bradford assay according to the manufacturer's instructions. After purification, they were dialyzed against DMEM at 4° C. until the dialysis was over 300×105 times the protein volume. aMTD/SD-fused TFF1 recombinant proteins purified under natural condition were analyzed on 15% SDS-PAGE gel and stained with Coomassie Brilliant Blue.

Solubility will be scored on a 5 point scale ranging from highly soluble proteins with little tendency to precipitate (+++++) to largely insoluble proteins (+) by measuring their turbidity (A450). Yield (mg/L) in physiological buffer condition of each recombinant protein will also be determined.

### Example 3

### Determination of Cell-Permeability of aMTD/SD-Fused TFF1 Recombinant Proteins

For quantitative cell-permeability, the aMTD-fused recombinant proteins were conjugated to FITC according to the manufacturer's instructions (Sigma-Aldrich, St. Louis, Mo.). RAW 264.7 cells were treated with 10 μM FITC-labeled recombinant proteins for 1 hour at 37° C., washed three times with cold PBS, and treated with proteinase K (10 μg/mL) for 5 minutes at 37° C. to remove cell surface-bound proteins. Cell-permeability of these recombinant proteins were analyzed by flow cytometry (Guava, Millipore, Darmstadt, Germany) using the FlowJo cytometric analysis software.

### Example 4

### Cell-Permeability and Intracellular Localization of aMTD/SD-Fused TFF1 Recombinant Proteins

For a visual reference of cell-permeability, NIH3T3 cells were cultured for 24 hours on coverslip in 24-wells chamber slides, treated with 10 μM FITC-conjugated recombinant proteins for 1 hour at 37° C., and washed three times with cold PBS. Treated cells were fixed in 4% paraformaldehyde (PFA, Junsei, Tokyo, Japan) for 10 minutes at room temperature, washed three times with PBS, and mounted with VECTASHIELD Mounting Medium (Vector laboratories, Burlingame, Calif.) with DAPI (4′,6-diamidino-2-phenylindole) for nuclear staining. The intracellular localization of the fluorescent signal was determined by confocal laser scanning microscopy (LM700, Zeiss, Germany).

### Example 5

### Systemic Delivery of CP-TFF1 In Vivo

ICR mice (6-week-old, female) were injected intraperitoneally (750 μg/head) with FITC only or FITC-conjugated CP-TFF1 recombinant proteins. After 2 hours, the liver, kidney, spleen, lung, heart, and brain were isolated, washed with an O.C.T. compound (Sakura), and frozen on dry ice. Cryosections (20 μm) were analyzed by fluorescence microscopy.

### Example 6

### Mechanism of aMTD-Mediated Intracellular Delivery

RAW264.7 cells were pretreated with different agents to assess the effect of various conditions on protein uptake: (i) 5 μg/ml proteinase K for 10 minutes, (ii) 20 μM Taxol for 30 minutes, (iii) 10 μM antimycin in the presence or absence of 1 mM ATP for 2 hours (iv) incubation on ice (or maintained at 37° C.) for 15, 30, or 60 minutes, and (v) 100 mM EDTA for 3 hours. These agents were used at concentrations known to be active in other applications. The cells were then incubated with 10 μM FITC-labeled proteins for 1 hour at 37° C., were washed three times with ice-cold PBS, treated with proteinase K (10 μg/ml for 5 minutes at 37° C.) to remove cell-surface bound proteins and analyzed by flow cytometry.

### Example 7

### Cell Proliferation Assay: CellTiter-Glo Cell Viability Assay

Cell viability assay was evaluated with Cell-Titer Glo luminescent cell viability assay. Various gastric cancer cell lines were treated with 10 μM CP-TFF1 recombinant proteins or buffer alone for 72 hours with 2% fetal bovine serum, and the luminescence was analyzed.

### Example 8

### Cell Migration Assay: Wound-Healing Assay

Cancer cell migration was determined using the wound healing assay. Briefly, cells were seeded into 12-well plates and grown to 90% confluence. The wounds were produced by scraping of the cell layer with a sterile white tip. For the CP-TFF1 recombinant protein treatment group, cells were treated with CP-TFF1 recombinant protein (10 μM) for 1 hour prior to changing the growth medium. Cells were cultured for an additional 24˜48 hours before being photographed. The migration is quantified by counting the number of cells that migrated from the wound edge into the clear area.

### Example 9

### Cell Migration Assay: Transwell Assay

The lower surface of Transwell inserts (Costar) was coated with gelatin (10 μg/ml), and the membranes were allowed to dry for 1 hour at room temperature. The Transwell inserts were assembled into a 24-well plate, and the lower chamber was filled with growth media containing 10% FBS and bFGF (40 ng/ml). CP-TFF1-treated AGS Cells (5×105) were added to each upper chamber, and the plate was incubated at 37° C. in a 5% CO2 incubator for 24 hours. Migrated cells were fixed with 4% paraformaldehyde for 10 minutes, stained with 0.1% (w/v) crystal violet for 1 hour and counted.

### Example 10

### Western Blot Analysis

For western blot analysis, CP-TFF1 (10 μM)-treated gastric cells were washed with PBS and were lysed in a lysis buffer (RIPA buffer) containing a protease cocktail (Roche) and phosphatase inhibitor cocktail. Equal amounts of cell lysate protein were subjected to SDS-PAGE and transferred to nitrocellulose membranes (BioRad). The protein transferred membranes were incubated to block non-specific binding sites in immersing the membrane in 5% non-fat dried milk (BD Bioscience). The membranes were incubated with cleaved Caspase-3 (1:1000) (Cell Signaling) overnight at 4° C. and β-actin at room temperature and then incubated with the appropriate horseradish peroxidase-conjugated secondary antibodies for 1 hour at room temperature. The blots were developed using a chemiluminescence detection system (ECL kit; Amersham Pharmacia Biotech) and exposed to an x-ray film (AGFA).

### Example 11

### Xenograft Animal Models

Female Balb/c nu/nu mice (DooYeol Biotech., Seoul, Korea) were subcutaneously implanted with MKN45 tumor block (2 mm3) into the left back side of the mouse. Tumor-bearing mice were intravenously administered with 800 μg/head the recombinant protein (HSAM165T1SB, HSAMT1SB) for 21 days and observed for 2 weeks following the termination of the treatment. Tumor size was monitored by measuring the longest (length) and shortest dimensions (width) once a day with a dial caliper, and tumor volume was calculated as width2×length×0.5.

### Example 12

### Immunohistochemistry (IHC)

Tissue samples were fixed in 4% Paraformaldehyde (Duksan) for 3 days, dehydrated, cleared with xylene and embedded in Paraplast. Sections (6 μm thick) of tumor were placed onto poly-L-lysine coated slides. To block endogenous peroxidase activity, sections were incubated for 15 minutes with 3% H2O2 in methanol. After washing three times with PBS, slides were incubated for 30 minutes with blocking solution (5% fetal bovine serum in PBS). Rabbit anti-VEGF (ab46154, abcam) were diluted 1:1000 (to protein concentration 0.1 μg/ml) in blocking solution, applied to sections, and incubated at 4° C. for 24 hours. After washing three times with PBS, sections were incubated with biotinylated mouse and rabbit IgG (Vector Laboratories) at a 1:1000 dilution for 1 hour at room temperature, then incubated with avidin-biotinylated peroxidase complex using a Vectorstain ABC Kit (Vector Laboratories) for 30 minutes at room temperature. After the slides are reacted with oxidized 3,3-diaminobenzidine as a chromogen, they were counterstained with Harris hematoxylin (Sigma-Aldrich). Permanently mounted slides were observed and photographed using a microscope equipped with a digital imaging system (ECLIPSE Ti, Nikon, Japan).

### Example 13

### Statistical Analysis

All data are presented as mean±s.d. Differences between groups were tested for statistical significance using Student's t-test and were considered significant at p<0.05 or p<0.01.

It will be apparent to those skilled in the art that various modifications can be made to the above-described exemplary embodiments of the present invention without departing from the spirit or scope of the invention. Thus, it is intended that the present invention covers all such modifications provided that they come within the scope of the appended claims and their equivalents.

SEQUENCE LISTING
[cDNA Sequence of Histidine Tag]
SEQ ID NO: 481
ATGGGCAGCAGCCATCATCATCATCATCACAGCAGCGGCCTGGTGCCGCGCGGCAGC
[Amino Acid Sequence of Histidine Tag]
SEQ ID NO: 482
MetGlySerSerHisHisHisHisHisHisSerSerGlyLeuValProArgGlySer
[cDNA Sequences of aMTDs and Peptides]
SEQ ID NO: 483
aMTD43: CTGCTGGCGGCGCCGCTGGTGGTGGCGGCGGTGCCG
[cDNA Sequences of aMTDs and Peptides]
SEQ ID NO: 484
aMTD165: GCGCTGGCGGTGCCGGTGGCGCTGGCGATTGTGCCG
[Amino Acid Sequences of aMTDs and Peptides]
SEQ ID NO: 485
aMTD43: LLAAPLVVAAVP
[Amino Acid Sequences of aMTDs and Peptides]
SEQ ID NO: 486
aMTD165: ALAVPVALAIVP
[cDNA Sequence of human TFF1]
SEQ ID NO: 487
ATGGCCACCATGGAGAACAAGGTGATCTGCGCCCTGGTCCTGGTGTCCATGCTGGCCCTCGG
CACCCTGGCCGAGGCCCAGACAGAGACGTGTACAGTGGCCCCCCGTGAAAGACAGAATTGTG
GTTTTCCTGGTGTCACGCCCTCCCAGTGTGCAAATAAGGGCTGCTGTTTCGACGACACCGTT
CGTGGGGTCCCCTGGTGCTTCTATCCTAATACCATCGACGTCCCTCCAGAAGAGGAGTGTGA
ATTT
[Amino Acid Sequence of human TFF1]
SEQ ID NO: 488
MetAlaThrMetGluAsnLysVallleCysAlaLeuValLeuValSerMetLeuAlaLeuGly
ThrLeuAlaGluAlaGlnThrGluThrCysThrValAlaProArgGluArgGlnAsnCysGly
PheProGlyValThrProSerGlnCysAlaAsnLysGlyCysCysPheAspAspThrVal
ArgGlyValProTrpCysPheTyrProAsnThrIleAspValProProGluGluGluCysGlu
Phe
[cDNA Sequences of SDA]
SEQ ID NO: 489
ATGGCAAATA TTACCGTTTT CTATAACGAA GACTTCCAGG GTAAGCAGGT
CGATCTGCCG
CCTGGCAACT ATACCCGCGC CCAGTTGGCG GCGCTGGGCA TCGAGAATAA
TACCATCAGC
TCGGTGAAGG TGCCGCCTGG CGTGAAGGCT ATCCTGTACC AGAACGATGG
TTTCGCCGGC
GACCAGATCG AAGTGGTGGC CAATGCCGAG GAGTTGGGCC CGCTGAATAA
TAACGTCTCC
AGCATCCGCG TCATCTCCGT GCCCGTGCAG CCGCGCATGG CAAATATTAC
CGTTTTCTAT
AACGAAGACT TCCAGGGTAA GCAGGTCGAT CTGCCGCCTG GCAACTATAC
CCGCGCCCAG
TTGGCGGCGC TGGGCATCGA GAATAATACC ATCAGCTCGG TGAAGGTGCC
GCCTGGCGTG
AAGGCTATCC TCTACCAGAA CGATGGTTTC GCCGGCGACC AGATCGAAGT
GGTGGCCAAT
GCCGAGGAGC TGGGTCCGCT GAATAATAAC GTCTCCAGCA TCCGCGTCAT
CTCCGTGCCG
GTGCAGCCGA GG
[Amino Acid Sequences of SDA]
SEQ ID NO: 490
Met Ala Asn Ile Thr Val Phe Tyr Asn Glu Asp Phe Gln Gly Lys
Gln Val Asp Leu Pro Pro Gly Asn Tyr Thr Arg Ala Gln Leu Ala
Ala Leu Gly Ile Glu Asn Asn Thr Ile Ser Ser Val Lys Val Pro
Pro Gly Val Lys Ala Ile Leu Tyr Gln Asn Asp Gly Phe Ala Gly
Asp Gln Ile Glu Val Val Ala Asn Ala Glu Glu Leu Gly Pro Leu
Asn Asn Asn Val Ser Ser Ile Arg Val Ile Ser Val Pro Val Gln
Pro Arg Met Ala Asn Ile Thr Val Phe Tyr Asn Glu Asp Phe Gln
Gly Lys Gln Val Asp Leu Pro Pro Gly Asn Tyr Thr Arg Ala Gln
Leu Ala Ala Leu Gly Ile Glu Asn Asn Thr Ile Ser Ser Val Lys
Val Pro Pro Gly Val Lys Ala Ile Leu Tyr Gln Asn Asp Gly Phe
Ala Gly Asp Gln Ile Glu Val Val Ala Asn Ala Glu Glu Leu Gly
Pro Leu Asn Asn Asn Val Ser Ser Ile Arg Val Ile Ser Val Pro
Val Gln Pro Arg
[cDNA Sequences of SDB]
SEQ ID NO: 491
ATGGCAGAAC AAAGCGACAA GGATGTGAAG TACTACACTC TGGAGGAGAT
TCAGAAGCAC
AAAGACAGCA AGAGCACCTG GGTGATCCTA CATCATAAGG TGTACGATCT
GACCAAGTTT
CTCGAAGAGC ATCCTGGTGG GGAAGAAGTC CTGGGCGAGC AAGCTGGGGG
TGATGCTACT
GAGAACTTTG AGGACGTCGG GCACTCTACG GATGCACGAG AACTGTCCAA
AACATACATC
ATCGGGGAGC TCCATCCAGA TGACAGATCA AAGATAGCCA AGCCTTCGGA
AACCCTT
[Amino Acid Sequences of SDB]
SEQ ID NO: 492
Met Ala Glu Gln Ser Asp Lys Asp Val Lys Tyr Tyr Thr Leu Glu
Glu Ile Gln Lys His Lys Asp Ser Lys Ser Thr Trp Val Ile Leu
His His Lys Val Tyr Asp Leu Thr Lys Phe Leu Glu Glu His Pro
Gly Gly Glu Glu Val Lue Gly Glu Gln Ala Gly Gly Asp Ala Thr
Glu Asn Phe Glu Asp Val Gly His Ser Thr Asp Ala Arg Glu Leu
Ser Lys Thr Tyr Ile Ile Gly Glu Leu His Pro Asp Asp Arg Ser
Lys Ile Ala Lys Pro Ser Glu Thr Lue
[cDNA Sequences of SDC]
SEQ ID NO: 493
ATGAGCGATA AAATTATTCA CCTGACTGAC GACAGTTTTG ACACGGATGT
ACTCAAAGCG
GACGGGGCGA TCCTCGTCGA TTTCTGGGCA GAGTGGTGCG GTCCGTGCAA
AATGATCGCC
CCGATTCTGG ATGAAATCGC TGACGAATAT CAGGGCAAAC TGACCGTTGC
AAAACTGAAC
ATCGATCAAA ACCCTGGCAC TGCGCCGAAA TATGGCATCC GTGGTATCCC
GACTCTGCTG
CTGTTCAAAA ACGGTGAAGT GGCGGCAACC AAAGTGGGTG CACTGTCTAA
AGGTCAGTTG
AAAGAGTTCC TCGACGCTAA
CCTGGCC
[Amino Acid Sequences of SDC]
SEQ ID NO: 494
Met Ser Asp Lys Ile Ile His Leu Thr Asp Asp Ser Phe Asp Thr
Asp Val Leu Lys Ala Asp Gly Ala Ile Leu Val Asp Phe Trp Ala
Glu Trp Cys Gly Pro Cys Lys Met Ile Ala Pro Ile Leu Asp Glu
Ile Ala Asp Glu Tyr Gln Gly Lys Leu Thr Val Ala Lys Leu Asn
Ile Asp Gln Asn Pro Gly Thr Ala Pro Lys Tyr Gly Ile Arg Gly
Ile Pro Thr Leu Leu Leu Phe Lys Asn Gly Glu Val Ala Ala Thr
Lys Val Gly Ala Leu Ser Lys Gly Gln Leu Lys Glu Phe Leu Asp
Ala Asn Leu Ala
[cDNA Sequences of SDD]
SEQ ID NO: 495
ATGAAAAAGA TTTGGCTGGC GCTGGCTGGT TTAGTTTTAG CGTTTAGCGC
ATCGGCGGCG
CAGTATGAAG ATGGTAAACA GTACACTACC CTGGAAAAAC CGGTAGCTGG
CGCGCCGCAA
GTGCTGGAGT TTTTCTCTTT CTTCTGCCCG CACTGCTATC AGTTTGAAGA
AGTTCTGCAT
ATTTCTGATA ATGTGAAGAA AAAACTGCCG GAAGGCGTGA AGATGACTAA
ATACCACGTC
AACTTCATGG GTGGTGACCT GGGCAAAGAT CTGACTCAGG CATGGGCTGT
GGCGATGGCG
CTGGGCGTGG AAGACAAAGT GACTGTTCCG CTGTTTGAAG GCGTACAGAA
AACCCAGACC
ATTCGTTCTG CTTCTGATAT CCGCGATGTA TTTATCAACG CAGGTATTAA
AGGTGAAGAG
TACGACGCGG CGTGGAACAG CTTCGTGGTG AAATCTCTGG TCGCTCAGCA
GGAAAAAGCT
GCAGCTGACG TGCAATTGCG TGGCGTTCCG GCGATGTTTG TTAACGGTAA
ATATCAGCTG
AATCCGCAGG GTATGGATAC CAGCAATATG GATGTTTTTG TTCAGCAGTA
TGCTGATACA
GTGAAATATC TGTCCGAGAA AAAA
[Amino Acid Sequences of SDD]
SEQ ID NO: 496
Met Lys Lys Ile Trp Leu Ala Leu Ala Gly Leu Val Leu Ala Phe
Ser Ala Ser Ala Ala Gln Tyr Glu Asp Gly Lys Gln Tyr Thr Thr
Leu Glu Lys Pro Val Ala Gly Ala Pro Gln Val Leu Glu Phe Phe
Ser Phe Phe Cys Pro His Cys Tyr Gln Phe Glu Glu Val Leu His
Ile Ser Asp Asn Val Lys Lys Lys Leu Pro Glu Gly Val Lys Met
Thr Lys Tyr His Val Asn Phe Met Gly Gly Asp Leu Gly Lys Asp
Leu Thr Gln Ala Trp Ala Val Ala Met Ala Leu Gly Val Glu Asp
Lys Val Thr Val Pro Leu Phe Glu Gly Val Gln Lys Thr Gln Thr
Ile Arg Ser Ala Ser Asp Ile Arg Asp Val Phe Ile Asn Ala Gly
Ile Lys Gly Glu Glu Tyr Asp Ala Ala Trp Asn Ser Phe Val Val
Lys Ser Leu Val Ala Gln Gln Glu Lys Ala Ala Ala Asp Val Gln
Leu Arg Gly Val Pro Ala Met Phe Val Asn Gly Lys Tyr Gln Leu
Asn Pro Gln Gly Met Asp Thr Ser Asn Met Asp Val Phe Val Gln
Gln Tyr Ala Asp Thr Val Lys Tyr Leu Ser Glu Lys Lys
[cDNA Sequences of SDE]
SEQ ID NO: 497
GGGTCCCTGC AGGACTCAGA AGTCAATCAA GAAGCTAAGC CAGAGGTCAA
GCCAGAAGTC AAGCCTGAGA CTCACATCAA TTTAAAGGTG TCCGATGGAT
CTTCAGAGAT CTTCTTCAAG ATCAAAAAGA CCACTCCTTT AAGAAGGCTG
ATGGAAGCGT TCGCTAAAAG ACAGGGTAAG GAAATGGACT CCTTAACGTT
CTTGTACGAC GGTATTGAAA TTCAAGCTGA TCAGACCCCT GAAGATTTGG
ACATGGAGGA TAACGATATT ATTGAGGCTC ACCGCGAACA GATTGGAGGT
[Amino Acid Sequences of SDE]
SEQ ID NO: 498
Gly Ser Leu Gln Asp Ser Glu Val Asn Gln Glu Ala Lys Pro Glu Val
Lys Pro Glu Val Lys Pro Glu Thr His Ile Asn Leu Lys Val Ser Asp
Gly Ser Ser Glu Ile Phe Phe Lys Ile Lys Lys Thr Thr Pro Leu Arg
Arg Leu Met Glu Ala Phe Ala Lys Arg Gln Gly Lys Glu Met Asp Ser
Leu Thr Phe Leu Tyr Asp Gly Ile Glu Ile Gln Ala Asp Gln Thr Pro
Glu Asp Leu Asp Met Glu Asp Asn Asp Ile Ile Glu Ala His Arg Glu
Gln Ile Gly Gly
[cDNA Sequences of SDF]
SEQ ID NO: 499
GGATCCGAAA TCGGTACTGG CTTTCCATTC GACCCCCATT ATGTGGAAGT
CCTGGGCGAG
CGCATGCACT ACGTCGATGT TGGTCCGCGC GATGGCACCC CTGTGCTGTT
CCTGCACGGT
AACCCGACCT CCTCCTACGT GTGGCGCAAC ATCATCCCGC ATGTTGCACC
GACCCATCGC
TGCATTGCTC CAGACCTGAT CGGTATGGGC AAATCCGACA AACCAGACCT
GGGTTATTTC
TTCGACGACC ACGTCCGCTT CATGGATGCC TTCATCGAAG CCCTGGGTCT
GGAAGAGGTC
GTCCTGGTCA TTCACGACTG GGGCTCCGCT CTGGGTTTCC ACTGGGCCAA
GCGCAATCCA
GAGCGCGTCA AAGGTATTGC ATTTATGGAG TTCATCCGCC CTATCCCGAC
CTGGGACGAA
TGGCCAGAAT TTGCCCGCGA GACCTTCCAG GCCTTCCGCA CCACCGACGT
CGGCCGCAAG
CTGATCATCG ATCAGAACGT TTTTATCGAG GGTACGCTGC CGATGGGTGT
CGTCCGCCCG
CTGACTGAAG TCGAGATGGA CCATTACCGC GAGCCGTTCC TGAATCCTGT
TGACCGCGAG
CCACTGTGGC GCTTCCCAAA CGAGCTGCCA ATCGCCGGTG AGCCAGCGAA
CATCGTCGCG
CTGGTCGAAG AATACATGGA CTGGCTGCAC CAGTCCCCTG TCCCGAAGCT
GCTGTTCTGG
GGCACCCCAG GCGTTCTGAT CCCACCGGCC GAAGCCGCTC GCCTGGCCAA
AAGCCTGCCT
AACTGCAAGG CTGTGGACAT CGGCCCGGGT CTGAATCTGC TGCAAGAAGA
CAACCCGGAC
CTGATCGGCA GCGAGATCGC GCGCTGGCTG TCTACTCTGG AGATTTCCGG
T
[Amino Acid Sequences of SDF]
SEQ ID NO: 500
Gly Ser Glu Ile Gly Thr Gly Phe Pro Phe Asp Pro His Tyr Val Glu
Val Leu Gly Glu Arg Met His Tyr Val Asp Val Gly Pro Arg Asp Gly
Thr Pro Val Leu Phe Leu His Gly Asn Pro Thr Ser Ser Tyr Val Trp
Arg Asn Ile Ile Pro His Val Ala Pro Thr His Arg Cys Ile Ala Pro
Asp Leu Ile Gly Met Gly Lys Ser Asp Lys Pro Asp Leu Gly Tyr Phe
Phe Asp Asp His Val Arg Phe Met Asp Ala Phe Ile Glu Ala Leu Gly
Leu Glu Glu Val Val Leu Val Ile His Asp Trp Gly Ser Ala Leu Gly
Phe His Trp Ala Lys Arg Asn Pro Glu Arg Val Lys Gly Ile Ala Phe
Met Glu Phe Ile Arg Pro Ile Pro Thr Trp Asp Glu Trp Pro Glu Phe
Ala Arg Glu Thr Phe Gln Ala Phe Arg Thr Thr Asp Val Gly Arg Lys
Leu Ile Ile Asp Gln Asn Val Phe Ile Glu Gly Thr Leu Pro Met Gly
Val Val Arg Pro Leu Thr Glu Val Glu Met Asp His Tyr Arg Glu Pro
Phe Leu Asn Pro Val Asp Arg Glu Pro Leu Trp Arg Phe Pro Asn Glu
Leu Pro Ile Ala Gly Glu Pro Ala Asn Ile Val Ala Leu Val Glu Glu
Tyr Met Asp Trp Leu His Gln Ser Pro Val Pro Lys Leu Leu Phe Trp
Gly Thr Pro Gly Val Leu Ile Pro Pro Ala Glu Ala Ala Arg Leu Ala
Lys Ser Leu Pro Asn Cys Lys Ala Val Asp Ile Gly Pro Gly Leu Asn
Leu Leu Gln Glu Asp Asn Pro Asp Leu Ile Gly Ser Glu Ile Ala Arg
Trp Leu Ser Thr Leu Glu Ile Ser Gly

What is claimed is:

1. TTFF1 recombinant proteins fused to newly invented hydrophobic cell-penetrating peptides (CPPs)-advanced macromolecule transduction domains (aMTDs) and Solubilization domains (SDs)

2. The TFF1 recombinant proteins according to claim 1, wherein aMTDs are selected from the group consisting of SEQ ID NO: 1 to SEQ ID NO: 240.

3. The TFF1 recombinant proteins according to claim 1, wherein SDs are selected from the group consisting of SEQ ID NO: 490, SEQ ID NO: 492, SEQ ID NO: 494, SEQ ID NO: 496, SEQ ID NO: 498, and SEQ ID NO: 500.

4. The TFF1 recombinant proteins according to claim 1, wherein SDs are fused to TFF1 recombinant proteins for high solubility and yield.

5. Isolated polynucleotides that encode that encode the TFF1 recombinant proteins according to claim 1.

6. The isolated polynucleotides according to claim 5, wherein the isolated polynucleotide of aMTDs are selected from the group consisting of SEQ ID NO: 241 to SEQ ID NO: 480.

7. The isolated polynucleotides according to claim 5, wherein the isolated polynucleotide of SDs are selected from the group consisting of SEQ ID NO: 489, SEQ ID NO: 491, SEQ ID NO: 493, SEQ ID NO: 495, SEQ ID NO: 497, and SEQ ID NO: 499.

8. The result of therapeutic applicability in gastric cancer with TFF1 recombinant proteins fused to newly invented hydrophobic cell-penetrating peptides (CPPs), namely advanced macromolecule transduction domains (aMTDs) and solubilization domain (SD)


SEQUENCE LISTING (500 sequences)

SEQ ID NO: 1 - protein, 12 aa, Artificial Sequence
  Advanced Macromolecule Transduction Domain (aMTD) Sequences for Improvement of Cell-Permeability
AAALAPVVLALP

SEQ ID NO: 2 - protein, 12 aa, Artificial Sequence
  Advanced Macromolecule Transduction Domain (aMTD) Sequences for Improvement of Cell-Permeability
AAAVPLLAVVVP

SEQ ID NO: 3 - protein, 12 aa, Artificial Sequence
  Advanced Macromolecule Transduction Domain (aMTD) Sequences for Improvement of Cell-Permeability
AALLVPAAVLAP

SEQ ID NO: 4 - protein, 12 aa, Artificial Sequence
  Advanced Macromolecule Transduction Domain (aMTD) Sequences for Improvement of Cell-Permeability
ALALLPVAALAP

SEQ ID NO: 5 - protein, 12 aa, Artificial Sequence
  Advanced Macromolecule Transduction Domain (aMTD) Sequences for Improvement of Cell-Permeability
AAALLPVALVAP

SEQ ID NO: 6 - protein, 12 aa, Artificial Sequence
  Advanced Macromolecule Transduction Domain (aMTD) Sequences for Improvement of Cell-Permeability
VVALAPALAALP

SEQ ID NO: 7 - protein, 12 aa, Artificial Sequence
  Advanced Macromolecule Transduction Domain (aMTD) Sequences for Improvement of Cell-Permeability
LLAAVPAVLLAP

SEQ ID NO: 8 - protein, 12 aa, Artificial Sequence
  Advanced Macromolecule Transduction Domain (aMTD) Sequences for Improvement of Cell-Permeability
AAALVPVVALLP

SEQ ID NO: 9 - protein, 12 aa, Artificial Sequence
  Advanced Macromolecule Transduction Domain (aMTD) Sequences for Improvement of Cell-Permeability
AVALLPALLAVP

SEQ ID NO: 10 - protein, 12 aa, Artificial Sequence
  Advanced Macromolecule Transduction Domain (aMTD) Sequences for Improvement of Cell-Permeability
AVVLVPVLAAAP

SEQ ID NO: 11 - protein, 12 aa, Artificial Sequence
  Advanced Macromolecule Transduction Domain (aMTD) Sequences for Improvement of Cell-Permeability
VVLVLPAAAAVP

SEQ ID NO: 12 - protein, 12 aa, Artificial Sequence
  Advanced Macromolecule Transduction Domain (aMTD) Sequences for Improvement of Cell-Permeability
IALAAPALIVAP

SEQ ID NO: 13 - protein, 12 aa, Artificial Sequence
  Advanced Macromolecule Transduction Domain (aMTD) Sequences for Improvement of Cell-Permeability
IVAVAPALVALP

SEQ ID NO: 14 - protein, 12 aa, Artificial Sequence
  Advanced Macromolecule Transduction Domain (aMTD) Sequences for Improvement of Cell-Permeability
VAALPVVAVVAP

SEQ ID NO: 15 - protein, 12 aa, Artificial Sequence
  Advanced Macromolecule Transduction Domain (aMTD) Sequences for Improvement of Cell-Permeability
LLAAPLVVAAVP

SEQ ID NO: 16 - protein, 12 aa, Artificial Sequence
  Advanced Macromolecule Transduction Domain (aMTD) Sequences for Improvement of Cell-Permeability
ALAVPVALLVAP

SEQ ID NO: 17 - protein, 12 aa, Artificial Sequence
  Advanced Macromolecule Transduction Domain (aMTD) Sequences for Improvement of Cell-Permeability
VAALPVLLAALP

SEQ ID NO: 18 - protein, 12 aa, Artificial Sequence
  Advanced Macromolecule Transduction Domain (aMTD) Sequences for Improvement of Cell-Permeability
VALLAPVALAVP

SEQ ID NO: 19 - protein, 12 aa, Artificial Sequence
  Advanced Macromolecule Transduction Domain (aMTD) Sequences for Improvement of Cell-Permeability
AALLVPALVAVP

SEQ ID NO: 20 - protein, 12 aa, Artificial Sequence
  Advanced Macromolecule Transduction Domain (aMTD) Sequences for Improvement of Cell-Permeability
AIVALPVAVLAP

SEQ ID NO: 21 - protein, 12 aa, Artificial Sequence
  Advanced Macromolecule Transduction Domain (aMTD) Sequences for Improvement of Cell-Permeability
IAIVAPVVALAP

SEQ ID NO: 22 - protein, 12 aa, Artificial Sequence
  Advanced Macromolecule Transduction Domain (aMTD) Sequences for Improvement of Cell-Permeability
AALLPALAALLP

SEQ ID NO: 23 - protein, 12 aa, Artificial Sequence
  Advanced Macromolecule Transduction Domain (aMTD) Sequences for Improvement of Cell-Permeability
AVVLAPVAAVLP

SEQ ID NO: 24 - protein, 12 aa, Artificial Sequence
  Advanced Macromolecule Transduction Domain (aMTD) Sequences for Improvement of Cell-Permeability
LAVAAPLALALP

SEQ ID NO: 25 - protein, 12 aa, Artificial Sequence
  Advanced Macromolecule Transduction Domain (aMTD) Sequences for Improvement of Cell-Permeability
AAVAAPLLLALP

SEQ ID NO: 26 - protein, 12 aa, Artificial Sequence
  Advanced Macromolecule Transduction Domain (aMTD) Sequences for Improvement of Cell-Permeability
LLVLPAAALAAP

SEQ ID NO: 27 - protein, 12 aa, Artificial Sequence
  Advanced Macromolecule Transduction Domain (aMTD) Sequences for Improvement of Cell-Permeability
LVALAPVAAVLP

SEQ ID NO: 28 - protein, 12 aa, Artificial Sequence
  Advanced Macromolecule Transduction Domain (aMTD) Sequences for Improvement of Cell-Permeability
LALAPAALALLP

SEQ ID NO: 29 - protein, 12 aa, Artificial Sequence
  Advanced Macromolecule Transduction Domain (aMTD) Sequences for Improvement of Cell-Permeability
ALIAAPILALAP

SEQ ID NO: 30 - protein, 12 aa, Artificial Sequence
  Advanced Macromolecule Transduction Domain (aMTD) Sequences for Improvement of Cell-Permeability
AVVAAPLVLALP

SEQ ID NO: 31 - protein, 12 aa, Artificial Sequence
  Advanced Macromolecule Transduction Domain (aMTD) Sequences for Improvement of Cell-Permeability
LLALAPAALLAP

SEQ ID NO: 32 - protein, 12 aa, Artificial Sequence
  Advanced Macromolecule Transduction Domain (aMTD) Sequences for Improvement of Cell-Permeability
AIVALPALALAP

SEQ ID NO: 33 - protein, 12 aa, Artificial Sequence
  Advanced Macromolecule Transduction Domain (aMTD) Sequences for Improvement of Cell-Permeability
AAIIVPAALLAP

SEQ ID NO: 34 - protein, 12 aa, Artificial Sequence
  Advanced Macromolecule Transduction Domain (aMTD) Sequences for Improvement of Cell-Permeability
IAVALPALIAAP

SEQ ID NO: 35 - protein, 12 aa, Artificial Sequence
  Advanced Macromolecule Transduction Domain (aMTD) Sequences for Improvement of Cell-Permeability
AVIVLPALAVAP

SEQ ID NO: 36 - protein, 12 aa, Artificial Sequence
  Advanced Macromolecule Transduction Domain (aMTD) Sequences for Improvement of Cell-Permeability
AVLAVPAVLVAP

SEQ ID NO: 37 - protein, 12 aa, Artificial Sequence
  Advanced Macromolecule Transduction Domain (aMTD) Sequences for Improvement of Cell-Permeability
VLAIVPAVALAP

SEQ ID NO: 38 - protein, 12 aa, Artificial Sequence
  Advanced Macromolecule Transduction Domain (aMTD) Sequences for Improvement of Cell-Permeability
LLAVVPAVALAP

SEQ ID NO: 39 - protein, 12 aa, Artificial Sequence
  Advanced Macromolecule Transduction Domain (aMTD) Sequences for Improvement of Cell-Permeability
AVIALPALIAAP

SEQ ID NO: 40 - protein, 12 aa, Artificial Sequence
  Advanced Macromolecule Transduction Domain (aMTD) Sequences for Improvement of Cell-Permeability
AVVALPAALIVP

SEQ ID NO: 41 - protein, 12 aa, Artificial Sequence
  Advanced Macromolecule Transduction Domain (aMTD) Sequences for Improvement of Cell-Permeability
LALVLPAALAAP

SEQ ID NO: 42 - protein, 12 aa, Artificial Sequence
  Advanced Macromolecule Transduction Domain (aMTD) Sequences for Improvement of Cell-Permeability
LAAVLPALLAAP

SEQ ID NO: 43 - protein, 12 aa, Artificial Sequence
  Advanced Macromolecule Transduction Domain (aMTD) Sequences for Improvement of Cell-Permeability
ALAVPVALAIVP

SEQ ID NO: 44 - protein, 12 aa, Artificial Sequence
  Advanced Macromolecule Transduction Domain (aMTD) Sequences for Improvement of Cell-Permeability
ALIAPVVALVAP

SEQ ID NO: 45 - protein, 12 aa, Artificial Sequence
  Advanced Macromolecule Transduction Domain (aMTD) Sequences for Improvement of Cell-Permeability
LLAAPVVIALAP

SEQ ID NO: 46 - protein, 12 aa, Artificial Sequence
  Advanced Macromolecule Transduction Domain (aMTD) Sequences for Improvement of Cell-Permeability
LAAIVPAIIAVP

SEQ ID NO: 47 - protein, 12 aa, Artificial Sequence
  Advanced Macromolecule Transduction Domain (aMTD) Sequences for Improvement of Cell-Permeability
AALVLPLIIAAP

SEQ ID NO: 48 - protein, 12 aa, Artificial Sequence
  Advanced Macromolecule Transduction Domain (aMTD) Sequences for Improvement of Cell-Permeability
LALAVPALAALP

SEQ ID NO: 49 - protein, 12 aa, Artificial Sequence
  Advanced Macromolecule Transduction Domain (aMTD) Sequences for Improvement of Cell-Permeability
LIAALPAVAALP

SEQ ID NO: 50 - protein, 12 aa, Artificial Sequence
  Advanced Macromolecule Transduction Domain (aMTD) Sequences for Improvement of Cell-Permeability
ALALVPAIAALP

SEQ ID NO: 51 - protein, 12 aa, Artificial Sequence
  Advanced Macromolecule Transduction Domain (aMTD) Sequences for Improvement of Cell-Permeability
AAILAPIVALAP

SEQ ID NO: 52 - protein, 12 aa, Artificial Sequence
  Advanced Macromolecule Transduction Domain (aMTD) Sequences for Improvement of Cell-Permeability
ALLIAPAAVIAP

SEQ ID NO: 53 - protein, 12 aa, Artificial Sequence
  Advanced Macromolecule Transduction Domain (aMTD) Sequences for Improvement of Cell-Permeability
AILAVPIAVVAP

SEQ ID NO: 54 - protein, 12 aa, Artificial Sequence
  Advanced Macromolecule Transduction Domain (aMTD) Sequences for Improvement of Cell-Permeability
ILAAVPIALAAP

SEQ ID NO: 55 - protein, 12 aa, Artificial Sequence
  Advanced Macromolecule Transduction Domain (aMTD) Sequences for Improvement of Cell-Permeability
VAALLPAAAVLP

SEQ ID NO: 56 - protein, 12 aa, Artificial Sequence
  Advanced Macromolecule Transduction Domain (aMTD) Sequences for Improvement of Cell-Permeability
AAAVVPVLLVAP

SEQ ID NO: 57 - protein, 12 aa, Artificial Sequence
  Advanced Macromolecule Transduction Domain (aMTD) Sequences for Improvement of Cell-Permeability
AALLVPALVAAP

SEQ ID NO: 58 - protein, 12 aa, Artificial Sequence
  Advanced Macromolecule Transduction Domain (aMTD) Sequences for Improvement of Cell-Permeability
AAVLLPVALAAP

SEQ ID NO: 59 - protein, 12 aa, Artificial Sequence
  Advanced Macromolecule Transduction Domain (aMTD) Sequences for Improvement of Cell-Permeability
AAALAPVLALVP

SEQ ID NO: 60 - protein, 12 aa, Artificial Sequence
  Advanced Macromolecule Transduction Domain (aMTD) Sequences for Improvement of Cell-Permeability
LVLVPLLAAAAP

SEQ ID NO: 61 - protein, 12 aa, Artificial Sequence
  Advanced Macromolecule Transduction Domain (aMTD) Sequences for Improvement of Cell-Permeability
ALIAVPAIIVAP

SEQ ID NO: 62 - protein, 12 aa, Artificial Sequence
  Advanced Macromolecule Transduction Domain (aMTD) Sequences for Improvement of Cell-Permeability
ALAVIPAAAILP

SEQ ID NO: 63 - protein, 12 aa, Artificial Sequence
  Advanced Macromolecule Transduction Domain (aMTD) Sequences for Improvement of Cell-Permeability
LAAAPVVIVIAP

SEQ ID NO: 64 - protein, 12 aa, Artificial Sequence
  Advanced Macromolecule Transduction Domain (aMTD) Sequences for Improvement of Cell-Permeability
VLAIAPLLAAVP

SEQ ID NO: 65 - protein, 12 aa, Artificial Sequence
  Advanced Macromolecule Transduction Domain (aMTD) Sequences for Improvement of Cell-Permeability
ALIVLPAAVAVP

SEQ ID NO: 66 - protein, 12 aa, Artificial Sequence
  Advanced Macromolecule Transduction Domain (aMTD) Sequences for Improvement of Cell-Permeability
VLAVAPALIVAP

SEQ ID NO: 67 - protein, 12 aa, Artificial Sequence
  Advanced Macromolecule Transduction Domain (aMTD) Sequences for Improvement of Cell-Permeability
AALLAPALIVAP

SEQ ID NO: 68 - protein, 12 aa, Artificial Sequence
  Advanced Macromolecule Transduction Domain (aMTD) Sequences for Improvement of Cell-Permeability
ALIAPAVALIVP

SEQ ID NO: 69 - protein, 12 aa, Artificial Sequence
  Advanced Macromolecule Transduction Domain (aMTD) Sequences for Improvement of Cell-Permeability
AIVLLPAAVVAP

SEQ ID NO: 70 - protein, 12 aa, Artificial Sequence
  Advanced Macromolecule Transduction Domain (aMTD) Sequences for Improvement of Cell-Permeability
VIAAPVLAVLAP

SEQ ID NO: 71 - protein, 12 aa, Artificial Sequence
  Advanced Macromolecule Transduction Domain (aMTD) Sequences for Improvement of Cell-Permeability
LALAPALALLAP

SEQ ID NO: 72 - protein, 12 aa, Artificial Sequence
  Advanced Macromolecule Transduction Domain (aMTD) Sequences for Improvement of Cell-Permeability
AIILAPIAAIAP

SEQ ID NO: 73 - protein, 12 aa, Artificial Sequence
  Advanced Macromolecule Transduction Domain (aMTD) Sequences for Improvement of Cell-Permeability
IALAAPILLAAP

SEQ ID NO: 74 - protein, 12 aa, Artificial Sequence
  Advanced Macromolecule Transduction Domain (aMTD) Sequences for Improvement of Cell-Permeability
IVAVALPALAVP

SEQ ID NO: 75 - protein, 12 aa, Artificial Sequence
  Advanced Macromolecule Transduction Domain (aMTD) Sequences for Improvement of Cell-Permeability
VVAIVLPALAAP

SEQ ID NO: 76 - protein, 12 aa, Artificial Sequence
  Advanced Macromolecule Transduction Domain (aMTD) Sequences for Improvement of Cell-Permeability
IVAVALPVALAP

SEQ ID NO: 77 - protein, 12 aa, Artificial Sequence
  Advanced Macromolecule Transduction Domain (aMTD) Sequences for Improvement of Cell-Permeability
IVAVALPAALVP

SEQ ID NO: 78 - protein, 12 aa, Artificial Sequence
  Advanced Macromolecule Transduction Domain (aMTD) Sequences for Improvement of Cell-Permeability
IVAVALPAVALP

SEQ ID NO: 79 - protein, 12 aa, Artificial Sequence
  Advanced Macromolecule Transduction Domain (aMTD) Sequences for Improvement of Cell-Permeability
IVAVALPAVLAP

SEQ ID NO: 80 - protein, 12 aa, Artificial Sequence
  Advanced Macromolecule Transduction Domain (aMTD) Sequences for Improvement of Cell-Permeability
VIVALAPAVLAP

SEQ ID NO: 81 - protein, 12 aa, Artificial Sequence
  Advanced Macromolecule Transduction Domain (aMTD) Sequences for Improvement of Cell-Permeability
IVAVALPALVAP

SEQ ID NO: 82 - protein, 12 aa, Artificial Sequence
  Advanced Macromolecule Transduction Domain (aMTD) Sequences for Improvement of Cell-Permeability
ALLIVAPVAVAP

SEQ ID NO: 83 - protein, 12 aa, Artificial Sequence
  Advanced Macromolecule Transduction Domain (aMTD) Sequences for Improvement of Cell-Permeability
AVVIVAPAVIAP

SEQ ID NO: 84 - protein, 12 aa, Artificial Sequence
  Advanced Macromolecule Transduction Domain (aMTD) Sequences for Improvement of Cell-Permeability
AVLAVAPALIVP

SEQ ID NO: 85 - protein, 12 aa, Artificial Sequence
  Advanced Macromolecule Transduction Domain (aMTD) Sequences for Improvement of Cell-Permeability
LVAAVAPALIVP

SEQ ID NO: 86 - protein, 12 aa, Artificial Sequence
  Advanced Macromolecule Transduction Domain (aMTD) Sequences for Improvement of Cell-Permeability
AVIVVAPALLAP

SEQ ID NO: 87 - protein, 12 aa, Artificial Sequence
  Advanced Macromolecule Transduction Domain (aMTD) Sequences for Improvement of Cell-Permeability
VVAIVLPAVAAP

SEQ ID NO: 88 - protein, 12 aa, Artificial Sequence
  Advanced Macromolecule Transduction Domain (aMTD) Sequences for Improvement of Cell-Permeability
AAALVIPAILAP

SEQ ID NO: 89 - protein, 12 aa, Artificial Sequence
  Advanced Macromolecule Transduction Domain (aMTD) Sequences for Improvement of Cell-Permeability
VIVALAPALLAP

SEQ ID NO: 90 - protein, 12 aa, Artificial Sequence
  Advanced Macromolecule Transduction Domain (aMTD) Sequences for Improvement of Cell-Permeability
VIVAIAPALLAP

SEQ ID NO: 91 - protein, 12 aa, Artificial Sequence
  Advanced Macromolecule Transduction Domain (aMTD) Sequences for Improvement of Cell-Permeability
IVAIAVPALVAP

SEQ ID NO: 92 - protein, 12 aa, Artificial Sequence
  Advanced Macromolecule Transduction Domain (aMTD) Sequences for Improvement of Cell-Permeability
AALAVIPAAILP

SEQ ID NO: 93 - protein, 12 aa, Artificial Sequence
  Advanced Macromolecule Transduction Domain (aMTD) Sequences for Improvement of Cell-Permeability
ALAAVIPAAILP

SEQ ID NO: 94 - protein, 12 aa, Artificial Sequence
  Advanced Macromolecule Transduction Domain (aMTD) Sequences for Improvement of Cell-Permeability
AAALVIPAAILP

SEQ ID NO: 95 - protein, 12 aa, Artificial Sequence
  Advanced Macromolecule Transduction Domain (aMTD) Sequences for Improvement of Cell-Permeability
LAAAVIPAAILP

SEQ ID NO: 96 - protein, 12 aa, Artificial Sequence
  Advanced Macromolecule Transduction Domain (aMTD) Sequences for Improvement of Cell-Permeability
LAAAVIPVAILP

SEQ ID NO: 97 - protein, 12 aa, Artificial Sequence
  Advanced Macromolecule Transduction Domain (aMTD) Sequences for Improvement of Cell-Permeability
AAILAAPLIAVP

SEQ ID NO: 98 - protein, 12 aa, Artificial Sequence
  Advanced Macromolecule Transduction Domain (aMTD) Sequences for Improvement of Cell-Permeability
VVAILAPLLAAP

SEQ ID NO: 99 - protein, 12 aa, Artificial Sequence
  Advanced Macromolecule Transduction Domain (aMTD) Sequences for Improvement of Cell-Permeability
AVVVAAPVLALP

SEQ ID NO: 100 - protein, 12 aa, Artificial Sequence
  Advanced Macromolecule Transduction Domain (aMTD) Sequences for Improvement of Cell-Permeability
AVVAIAPVLALP

SEQ ID NO: 101 - protein, 12 aa, Artificial Sequence
  Advanced Macromolecule Transduction Domain (aMTD) Sequences for Improvement of Cell-Permeability
ALAALVPAVLVP

SEQ ID NO: 102 - protein, 12 aa, Artificial Sequence
  Advanced Macromolecule Transduction Domain (aMTD) Sequences for Improvement of Cell-Permeability
ALAALVPVALVP

SEQ ID NO: 103 - protein, 12 aa, Artificial Sequence
  Advanced Macromolecule Transduction Domain (aMTD) Sequences for Improvement of Cell-Permeability
LAAALVPVALVP

SEQ ID NO: 104 - protein, 12 aa, Artificial Sequence
  Advanced Macromolecule Transduction Domain (aMTD) Sequences for Improvement of Cell-Permeability
ALAALVPALVVP

SEQ ID NO: 105 - protein, 12 aa, Artificial Sequence
  Advanced Macromolecule Transduction Domain (aMTD) Sequences for Improvement of Cell-Permeability
IAAVIVPAVALP

SEQ ID NO: 106 - protein, 12 aa, Artificial Sequence
  Advanced Macromolecule Transduction Domain (aMTD) Sequences for Improvement of Cell-Permeability
IAAVLVPAVALP

SEQ ID NO: 107 - protein, 12 aa, Artificial Sequence
  Advanced Macromolecule Transduction Domain (aMTD) Sequences for Improvement of Cell-Permeability
AVAILVPLLAAP

SEQ ID NO: 108 - protein, 12 aa, Artificial Sequence
  Advanced Macromolecule Transduction Domain (aMTD) Sequences for Improvement of Cell-Permeability
AVVILVPLAAAP

SEQ ID NO: 109 - protein, 12 aa, Artificial Sequence
  Advanced Macromolecule Transduction Domain (aMTD) Sequences for Improvement of Cell-Permeability
IAAVIVPVAALP

SEQ ID NO: 110 - protein, 12 aa, Artificial Sequence
  Advanced Macromolecule Transduction Domain (aMTD) Sequences for Improvement of Cell-Permeability
AIAIAIVPVALP

SEQ ID NO: 111 - protein, 12 aa, Artificial Sequence
  Advanced Macromolecule Transduction Domain (aMTD) Sequences for Improvement of Cell-Permeability
ILAVAAIPVAVP

SEQ ID NO: 112 - protein, 12 aa, Artificial Sequence
  Advanced Macromolecule Transduction Domain (aMTD) Sequences for Improvement of Cell-Permeability
ILAAAIIPAALP

SEQ ID NO: 113 - protein, 12 aa, Artificial Sequence
  Advanced Macromolecule Transduction Domain (aMTD) Sequences for Improvement of Cell-Permeability
LAVVLAAPAIVP

SEQ ID NO: 114 - protein, 12 aa, Artificial Sequence
  Advanced Macromolecule Transduction Domain (aMTD) Sequences for Improvement of Cell-Permeability
AILAAIVPLAVP

SEQ ID NO: 115 - protein, 12 aa, Artificial Sequence
  Advanced Macromolecule Transduction Domain (aMTD) Sequences for Improvement of Cell-Permeability
VIVALAVPALAP

SEQ ID NO: 116 - protein, 12 aa, Artificial Sequence
  Advanced Macromolecule Transduction Domain (aMTD) Sequences for Improvement of Cell-Permeability
AIVALAVPVLAP

SEQ ID NO: 117 - protein, 12 aa, Artificial Sequence
  Advanced Macromolecule Transduction Domain (aMTD) Sequences for Improvement of Cell-Permeability
AAIIIVLPAALP

SEQ ID NO: 118 - protein, 12 aa, Artificial Sequence
  Advanced Macromolecule Transduction Domain (aMTD) Sequences for Improvement of Cell-Permeability
LIVALAVPALAP

SEQ ID NO: 119 - protein, 12 aa, Artificial Sequence
  Advanced Macromolecule Transduction Domain (aMTD) Sequences for Improvement of Cell-Permeability
AIIIVIAPAAAP

SEQ ID NO: 120 - protein, 12 aa, Artificial Sequence
  Advanced Macromolecule Transduction Domain (aMTD) Sequences for Improvement of Cell-Permeability
LAALIVVPAVAP

SEQ ID NO: 121 - protein, 12 aa, Artificial Sequence
  Advanced Macromolecule Transduction Domain (aMTD) Sequences for Improvement of Cell-Permeability
ALLVIAVPAVAP

SEQ ID NO: 122 - protein, 12 aa, Artificial Sequence
  Advanced Macromolecule Transduction Domain (aMTD) Sequences for Improvement of Cell-Permeability
AVALIVVPALAP

SEQ ID NO: 123 - protein, 12 aa, Artificial Sequence
  Advanced Macromolecule Transduction Domain (aMTD) Sequences for Improvement of Cell-Permeability
ALAIVVAPVAVP

SEQ ID NO: 124 - protein, 12 aa, Artificial Sequence
  Advanced Macromolecule Transduction Domain (aMTD) Sequences for Improvement of Cell-Permeability
LLALIIAPAAAP

SEQ ID NO: 125 - protein, 12 aa, Artificial Sequence
  Advanced Macromolecule Transduction Domain (aMTD) Sequences for Improvement of Cell-Permeability
ALALIIVPAVAP

SEQ ID NO: 126 - protein, 12 aa, Artificial Sequence
  Advanced Macromolecule Transduction Domain (aMTD) Sequences for Improvement of Cell-Permeability
LLAALIAPAALP

SEQ ID NO: 127 - protein, 12 aa, Artificial Sequence
  Advanced Macromolecule Transduction Domain (aMTD) Sequences for Improvement of Cell-Permeability
IVALIVAPAAVP

SEQ ID NO: 128 - protein, 12 aa, Artificial Sequence
  Advanced Macromolecule Transduction Domain (aMTD) Sequences for Improvement of Cell-Permeability
VVLVLAAPAAVP

SEQ ID NO: 129 - protein, 12 aa, Artificial Sequence
  Advanced Macromolecule Transduction Domain (aMTD) Sequences for Improvement of Cell-Permeability
AAVAIVLPAVVP

SEQ ID NO: 130 - protein, 12 aa, Artificial Sequence
  Advanced Macromolecule Transduction Domain (aMTD) Sequences for Improvement of Cell-Permeability
ALIAAIVPALVP

SEQ ID NO: 131 - protein, 12 aa, Artificial Sequence
  Advanced Macromolecule Transduction Domain (aMTD) Sequences for Improvement of Cell-Permeability
ALAVIVVPALAP

SEQ ID NO: 132 - protein, 12 aa, Artificial Sequence
  Advanced Macromolecule Transduction Domain (aMTD) Sequences for Improvement of Cell-Permeability
VAIALIVPALAP

SEQ ID NO: 133 - protein, 12 aa, Artificial Sequence
  Advanced Macromolecule Transduction Domain (aMTD) Sequences for Improvement of Cell-Permeability
VAIVLVAPAVAP

SEQ ID NO: 134 - protein, 12 aa, Artificial Sequence
  Advanced Macromolecule Transduction Domain (aMTD) Sequences for Improvement of Cell-Permeability
VAVALIVPALAP

SEQ ID NO: 135 - protein, 12 aa, Artificial Sequence
  Advanced Macromolecule Transduction Domain (aMTD) Sequences for Improvement of Cell-Permeability
AVILALAPIVAP

SEQ ID NO: 136 - protein, 12 aa, Artificial Sequence
  Advanced Macromolecule Transduction Domain (aMTD) Sequences for Improvement of Cell-Permeability
ALIVAIAPALVP

SEQ ID NO: 137 - protein, 12 aa, Artificial Sequence
  Advanced Macromolecule Transduction Domain (aMTD) Sequences for Improvement of Cell-Permeability
AAILIAVPIAAP

SEQ ID NO: 138 - protein, 12 aa, Artificial Sequence
  Advanced Macromolecule Transduction Domain (aMTD) Sequences for Improvement of Cell-Permeability
VIVALAAPVLAP

SEQ ID NO: 139 - protein, 12 aa, Artificial Sequence
  Advanced Macromolecule Transduction Domain (aMTD) Sequences for Improvement of Cell-Permeability
VLVALAAPVIAP

SEQ ID NO: 140 - protein, 12 aa, Artificial Sequence
  Advanced Macromolecule Transduction Domain (aMTD) Sequences for Improvement of Cell-Permeability
VALIAVAPAVVP

SEQ ID NO: 141 - protein, 12 aa, Artificial Sequence
  Advanced Macromolecule Transduction Domain (aMTD) Sequences for Improvement of Cell-Permeability
VIAAVLAPVAVP

SEQ ID NO: 142 - protein, 12 aa, Artificial Sequence
  Advanced Macromolecule Transduction Domain (aMTD) Sequences for Improvement of Cell-Permeability
ALIVLAAPVAVP

SEQ ID NO: 143 - protein, 12 aa, Artificial Sequence
  Advanced Macromolecule Transduction Domain (aMTD) Sequences for Improvement of Cell-Permeability
VAAAIALPAIVP

SEQ ID NO: 144 - protein, 12 aa, Artificial Sequence
  Advanced Macromolecule Transduction Domain (aMTD) Sequences for Improvement of Cell-Permeability
ILAAAAAPLIVP

SEQ ID NO: 145 - protein, 12 aa, Artificial Sequence
  Advanced Macromolecule Transduction Domain (aMTD) Sequences for Improvement of Cell-Permeability
LALVLAAPAIVP

SEQ ID NO: 146 - protein, 12 aa, Artificial Sequence
  Advanced Macromolecule Transduction Domain (aMTD) Sequences for Improvement of Cell-Permeability
ALAVVALPAIVP

SEQ ID NO: 147 - protein, 12 aa, Artificial Sequence
  Advanced Macromolecule Transduction Domain (aMTD) Sequences for Improvement of Cell-Permeability
AAILAPIVAALP

SEQ ID NO: 148 - protein, 12 aa, Artificial Sequence
  Advanced Macromolecule Transduction Domain (aMTD) Sequences for Improvement of Cell-Permeability
ILIAIAIPAAAP

SEQ ID NO: 149 - protein, 12 aa, Artificial Sequence
  Advanced Macromolecule Transduction Domain (aMTD) Sequences for Improvement of Cell-Permeability
LAIVLAAPVAVP

SEQ ID NO: 150 - protein, 12 aa, Artificial Sequence
  Advanced Macromolecule Transduction Domain (aMTD) Sequences for Improvement of Cell-Permeability
AAIAIIAPAIVP

SEQ ID NO: 151 - protein, 12 aa, Artificial Sequence
  Advanced Macromolecule Transduction Domain (aMTD) Sequences for Improvement of Cell-Permeability
LAVAIVAPALVP

SEQ ID NO: 152 - protein, 12 aa, Artificial Sequence
  Advanced Macromolecule Transduction Domain (aMTD) Sequences for Improvement of Cell-Permeability
LAIVLAAPAVLP

SEQ ID NO: 153 - protein, 12 aa, Artificial Sequence
  Advanced Macromolecule Transduction Domain (aMTD) Sequences for Improvement of Cell-Permeability
AAIVLALPAVLP

SEQ ID NO: 154 - protein, 12 aa, Artificial Sequence
  Advanced Macromolecule Transduction Domain (aMTD) Sequences for Improvement of Cell-Permeability
ALLVAVLPAALP

SEQ ID NO: 155 - protein, 12 aa, Artificial Sequence
  Advanced Macromolecule Transduction Domain (aMTD) Sequences for Improvement of Cell-Permeability
AALVAVLPVALP

SEQ ID NO: 156 - protein, 12 aa, Artificial Sequence
  Advanced Macromolecule Transduction Domain (aMTD) Sequences for Improvement of Cell-Permeability
AILAVALPLLAP

SEQ ID NO: 157 - protein, 12 aa, Artificial Sequence
  Advanced Macromolecule Transduction Domain (aMTD) Sequences for Improvement of Cell-Permeability
IVAVALVPALAP

SEQ ID NO: 158 - protein, 12 aa, Artificial Sequence
  Advanced Macromolecule Transduction Domain (aMTD) Sequences for Improvement of Cell-Permeability
IVAVALLPALAP

SEQ ID NO: 159 - protein, 12 aa, Artificial Sequence
  Advanced Macromolecule Transduction Domain (aMTD) Sequences for Improvement of Cell-Permeability
IVAVALLPAVAP

SEQ ID NO: 160 - protein, 12 aa, Artificial Sequence
  Advanced Macromolecule Transduction Domain (aMTD) Sequences for Improvement of Cell-Permeability
IVALAVLPAVAP

SEQ ID NO: 161 - protein, 12 aa, Artificial Sequence
  Advanced Macromolecule Transduction Domain (aMTD) Sequences for Improvement of Cell-Permeability
VAVLAVLPALAP

SEQ ID NO: 162 - protein, 12 aa, Artificial Sequence
  Advanced Macromolecule Transduction Domain (aMTD) Sequences for Improvement of Cell-Permeability
IAVLAVAPAVLP

SEQ ID NO: 163 - protein, 12 aa, Artificial Sequence
  Advanced Macromolecule Transduction Domain (aMTD) Sequences for Improvement of Cell-Permeability
LAVAIIAPAVAP

SEQ ID NO: 164 - protein, 12 aa, Artificial Sequence
  Advanced Macromolecule Transduction Domain (aMTD) Sequences for Improvement of Cell-Permeability
VALAIALPAVLP

SEQ ID NO: 165 - protein, 12 aa, Artificial Sequence
  Advanced Macromolecule Transduction Domain (aMTD) Sequences for Improvement of Cell-Permeability
AIAIALVPVALP

SEQ ID NO: 166 - protein, 12 aa, Artificial Sequence
  Advanced Macromolecule Transduction Domain (aMTD) Sequences for Improvement of Cell-Permeability
AAVVIVAPVALP

SEQ ID NO: 167 - protein, 12 aa, Artificial Sequence
  Advanced Macromolecule Transduction Domain (aMTD) Sequences for Improvement of Cell-Permeability
VAIIVVAPALAP

SEQ ID NO: 168 - protein, 12 aa, Artificial Sequence
  Advanced Macromolecule Transduction Domain (aMTD) Sequences for Improvement of Cell-Permeability
VALLAIAPALAP

SEQ ID NO: 169 - protein, 12 aa, Artificial Sequence
  Advanced Macromolecule Transduction Domain (aMTD) Sequences for Improvement of Cell-Permeability
VAVLIAVPALAP

SEQ ID NO: 170 - protein, 12 aa, Artificial Sequence
  Advanced Macromolecule Transduction Domain (aMTD) Sequences for Improvement of Cell-Permeability
AVALAVLPAVVP

SEQ ID NO: 171 - protein, 12 aa, Artificial Sequence
  Advanced Macromolecule Transduction Domain (aMTD) Sequences for Improvement of Cell-Permeability
AVALAVVPAVLP

SEQ ID NO: 172 - protein, 12 aa, Artificial Sequence
  Advanced Macromolecule Transduction Domain (aMTD) Sequences for Improvement of Cell-Permeability
IVVIAVAPAVAP

SEQ ID NO: 173 - protein, 12 aa, Artificial Sequence
  Advanced Macromolecule Transduction Domain (aMTD) Sequences for Improvement of Cell-Permeability
IVVAAVVPALAP

SEQ ID NO: 174 - protein, 12 aa, Artificial Sequence
  Advanced Macromolecule Transduction Domain (aMTD) Sequences for Improvement of Cell-Permeability
IVALVPAVAIAP

SEQ ID NO: 175 - protein, 12 aa, Artificial Sequence
  Advanced Macromolecule Transduction Domain (aMTD) Sequences for Improvement of Cell-Permeability
VAALPAVALVVP

SEQ ID NO: 176 - protein, 12 aa, Artificial Sequence
  Advanced Macromolecule Transduction Domain (aMTD) Sequences for Improvement of Cell-Permeability
LVAIAPLAVLAP

SEQ ID NO: 177 - protein, 12 aa, Artificial Sequence
  Advanced Macromolecule Transduction Domain (aMTD) Sequences for Improvement of Cell-Permeability
AVALVPVIVAAP

SEQ ID NO: 178 - protein, 12 aa, Artificial Sequence
  Advanced Macromolecule Transduction Domain (aMTD) Sequences for Improvement of Cell-Permeability
AIAVAIAPVALP

SEQ ID NO: 179 - protein, 12 aa, Artificial Sequence
  Advanced Macromolecule Transduction Domain (aMTD) Sequences for Improvement of Cell-Permeability
AIALAVPVLALP

SEQ ID NO: 180 - protein, 12 aa, Artificial Sequence
  Advanced Macromolecule Transduction Domain (aMTD) Sequences for Improvement of Cell-Permeability
LVLIAAAPIALP

SEQ ID NO: 181 - protein, 12 aa, Artificial Sequence
  Advanced Macromolecule Transduction Domain (aMTD) Sequences for Improvement of Cell-Permeability
LVALAVPAAVLP

SEQ ID NO: 182 - protein, 12 aa, Artificial Sequence
  Advanced Macromolecule Transduction Domain (aMTD) Sequences for Improvement of Cell-Permeability
AVALAVPALVLP

SEQ ID NO: 183 - protein, 12 aa, Artificial Sequence
  Advanced Macromolecule Transduction Domain (aMTD) Sequences for Improvement of Cell-Permeability
LVVLAAAPLAVP

SEQ ID NO: 184 - protein, 12 aa, Artificial Sequence
  Advanced Macromolecule Transduction Domain (aMTD) Sequences for Improvement of Cell-Permeability
LIVLAAPALAAP

SEQ ID NO: 185 - protein, 12 aa, Artificial Sequence
  Advanced Macromolecule Transduction Domain (aMTD) Sequences for Improvement of Cell-Permeability
VIVLAAPALAAP

SEQ ID NO: 186 - protein, 12 aa, Artificial Sequence
  Advanced Macromolecule Transduction Domain (aMTD) Sequences for Improvement of Cell-Permeability
AVVLAVPALAVP

SEQ ID NO: 187 - protein, 12 aa, Artificial Sequence
  Advanced Macromolecule Transduction Domain (aMTD) Sequences for Improvement of Cell-Permeability
LIIVAAAPAVAP

SEQ ID NO: 188 - protein, 12 aa, Artificial Sequence
  Advanced Macromolecule Transduction Domain (aMTD) Sequences for Improvement of Cell-Permeability
IVAVIVAPAVAP

SEQ ID NO: 189 - protein, 12 aa, Artificial Sequence
  Advanced Macromolecule Transduction Domain (aMTD) Sequences for Improvement of Cell-Permeability
LVALAAPIIAVP

SEQ ID NO: 190 - protein, 12 aa, Artificial Sequence
  Advanced Macromolecule Transduction Domain (aMTD) Sequences for Improvement of Cell-Permeability
IAAVLAAPALVP

SEQ ID NO: 191 - protein, 12 aa, Artificial Sequence
  Advanced Macromolecule Transduction Domain (aMTD) Sequences for Improvement of Cell-Permeability
IALLAAPIIAVP

SEQ ID NO: 192 - protein, 12 aa, Artificial Sequence
  Advanced Macromolecule Transduction Domain (aMTD) Sequences for Improvement of Cell-Permeability
AALALVAPVIVP

SEQ ID NO: 193 - protein, 12 aa, Artificial Sequence
  Advanced Macromolecule Transduction Domain (aMTD) Sequences for Improvement of Cell-Permeability
IALVAAPVALVP

SEQ ID NO: 194 - protein, 12 aa, Artificial Sequence
  Advanced Macromolecule Transduction Domain (aMTD) Sequences for Improvement of Cell-Permeability
IIVAVAPAAIVP

SEQ ID NO: 195 - protein, 12 aa, Artificial Sequence
  Advanced Macromolecule Transduction Domain (aMTD) Sequences for Improvement of Cell-Permeability
AVAAIVPVIVAP

SEQ ID NO: 196 - protein, 12 aa, Artificial Sequence
  Advanced Macromolecule Transduction Domain (aMTD) Sequences for Improvement of Cell-Permeability
AVLVLVAPAAAP

SEQ ID NO: 197 - protein, 12 aa, Artificial Sequence
  Advanced Macromolecule Transduction Domain (aMTD) Sequences for Improvement of Cell-Permeability
VVALLAPLIAAP

SEQ ID NO: 198 - protein, 12 aa, Artificial Sequence
  Advanced Macromolecule Transduction Domain (aMTD) Sequences for Improvement of Cell-Permeability
AAVVIAPLLAVP

SEQ ID NO: 199 - protein, 12 aa, Artificial Sequence
  Advanced Macromolecule Transduction Domain (aMTD) Sequences for Improvement of Cell-Permeability
IAVAVAAPLLVP

SEQ ID NO: 200 - protein, 12 aa, Artificial Sequence
  Advanced Macromolecule Transduction Domain (aMTD) Sequences for Improvement of Cell-Permeability
LVAIVVLPAVAP

SEQ ID NO: 201 - protein, 12 aa, Artificial Sequence
  Advanced Macromolecule Transduction Domain (aMTD) Sequences for Improvement of Cell-Permeability
AVAIVVLPAVAP

SEQ ID NO: 202 - protein, 12 aa, Artificial Sequence
  Advanced Macromolecule Transduction Domain (aMTD) Sequences for Improvement of Cell-Permeability
AVILLAPLIAAP

SEQ ID NO: 203 - protein, 12 aa, Artificial Sequence
  Advanced Macromolecule Transduction Domain (aMTD) Sequences for Improvement of Cell-Permeability
LVIALAAPVALP

SEQ ID NO: 204 - protein, 12 aa, Artificial Sequence
  Advanced Macromolecule Transduction Domain (aMTD) Sequences for Improvement of Cell-Permeability
VLAVVLPAVALP

SEQ ID NO: 205 - protein, 12 aa, Artificial Sequence
  Advanced Macromolecule Transduction Domain (aMTD) Sequences for Improvement of Cell-Permeability
VLAVAAPAVLLP

SEQ ID NO: 206 - protein, 12 aa, Artificial Sequence
  Advanced Macromolecule Transduction Domain (aMTD) Sequences for Improvement of Cell-Permeability
AAVVLLPIIAAP

SEQ ID NO: 207 - protein, 12 aa, Artificial Sequence
  Advanced Macromolecule Transduction Domain (aMTD) Sequences for Improvement of Cell-Permeability
ALLVIAPAIAVP

SEQ ID NO: 208 - protein, 12 aa, Artificial Sequence
  Advanced Macromolecule Transduction Domain (aMTD) Sequences for Improvement of Cell-Permeability
AVLVIAVPAIAP

SEQ ID NO: 209 - protein, 12 aa, Artificial Sequence
  Advanced Macromolecule Transduction Domain (aMTD) Sequences for Improvement of Cell-Permeability
ALLVVIAPLAAP

SEQ ID NO: 210 - protein, 12 aa, Artificial Sequence
  Advanced Macromolecule Transduction Domain (aMTD) Sequences for Improvement of Cell-Permeability
VLVAAILPAAIP

SEQ ID NO: 211 - protein, 12 aa, Artificial Sequence
  Advanced Macromolecule Transduction Domain (aMTD) Sequences for Improvement of Cell-Permeability
VLVAAVLPIAAP

SEQ ID NO: 212 - protein, 12 aa, Artificial Sequence
  Advanced Macromolecule Transduction Domain (aMTD) Sequences for Improvement of Cell-Permeability
VLAAAVLPLVVP

SEQ ID NO: 213 - protein, 12 aa, Artificial Sequence
  Advanced Macromolecule Transduction Domain (aMTD) Sequences for Improvement of Cell-Permeability
AIAIVVPAVAVP

SEQ ID NO: 214 - protein, 12 aa, Artificial Sequence
  Advanced Macromolecule Transduction Domain (aMTD) Sequences for Improvement of Cell-Permeability
VAIIAVPAVVAP

SEQ ID NO: 215 - protein, 12 aa, Artificial Sequence
  Advanced Macromolecule Transduction Domain (aMTD) Sequences for Improvement of Cell-Permeability
IVALVAPAAVVP

SEQ ID NO: 216 - protein, 12 aa, Artificial Sequence
  Advanced Macromolecule Transduction Domain (aMTD) Sequences for Improvement of Cell-Permeability
AAIVLLPAVVVP

SEQ ID NO: 217 - protein, 12 aa, Artificial Sequence
  Advanced Macromolecule Transduction Domain (aMTD) Sequences for Improvement of Cell-Permeability
AALIVVPAVAVP

SEQ ID NO: 218 - protein, 12 aa, Artificial Sequence
  Advanced Macromolecule Transduction Domain (aMTD) Sequences for Improvement of Cell-Permeability
AIALVVPAVAVP

SEQ ID NO: 219 - protein, 12 aa, Artificial Sequence
  Advanced Macromolecule Transduction Domain (aMTD) Sequences for Improvement of Cell-Permeability
LAIVPAAIAALP

SEQ ID NO: 220 - protein, 12 aa, Artificial Sequence
  Advanced Macromolecule Transduction Domain (aMTD) Sequences for Improvement of Cell-Permeability
LVAIAPAVAVLP

SEQ ID NO: 221 - protein, 12 aa, Artificial Sequence
  Advanced Macromolecule Transduction Domain (aMTD) Sequences for Improvement of Cell-Permeability
VLAVAPAVAVLP

SEQ ID NO: 222 - protein, 12 aa, Artificial Sequence
  Advanced Macromolecule Transduction Domain (aMTD) Sequences for Improvement of Cell-Permeability
ILAVVAIPAAAP

SEQ ID NO: 223 - protein, 12 aa, Artificial Sequence
  Advanced Macromolecule Transduction Domain (aMTD) Sequences for Improvement of Cell-Permeability
ILVAAAPIAALP

SEQ ID NO: 224 - protein, 12 aa, Artificial Sequence
  Advanced Macromolecule Transduction Domain (aMTD) Sequences for Improvement of Cell-Permeability
ILAVAAIPAALP

SEQ ID NO: 225 - protein, 12 aa, Artificial Sequence
  Advanced Macromolecule Transduction Domain (aMTD) Sequences for Improvement of Cell-Permeability
VIAIPAILAAAP

SEQ ID NO: 226 - protein, 12 aa, Artificial Sequence
  Advanced Macromolecule Transduction Domain (aMTD) Sequences for Improvement of Cell-Permeability
AIIIVVPAIAAP

SEQ ID NO: 227 - protein, 12 aa, Artificial Sequence
  Advanced Macromolecule Transduction Domain (aMTD) Sequences for Improvement of Cell-Permeability
AILIVVAPIAAP

SEQ ID NO: 228 - protein, 12 aa, Artificial Sequence
  Advanced Macromolecule Transduction Domain (aMTD) Sequences for Improvement of Cell-Permeability
AVIVPVAIIAAP

SEQ ID NO: 229 - protein, 12 aa, Artificial Sequence
  Advanced Macromolecule Transduction Domain (aMTD) Sequences for Improvement of Cell-Permeability
AVVIALPAVVAP

SEQ ID NO: 230 - protein, 12 aa, Artificial Sequence
  Advanced Macromolecule Transduction Domain (aMTD) Sequences for Improvement of Cell-Permeability
ALVAVIAPVVAP

SEQ ID NO: 231 - protein, 12 aa, Artificial Sequence
  Advanced Macromolecule Transduction Domain (aMTD) Sequences for Improvement of Cell-Permeability
ALVAVLPAVAVP

SEQ ID NO: 232 - protein, 12 aa, Artificial Sequence
  Advanced Macromolecule Transduction Domain (aMTD) Sequences for Improvement of Cell-Permeability
ALVAPLLAVAVP

SEQ ID NO: 233 - protein, 12 aa, Artificial Sequence
  Advanced Macromolecule Transduction Domain (aMTD) Sequences for Improvement of Cell-Permeability
AVLAVVAPVVAP

SEQ ID NO: 234 - protein, 12 aa, Artificial Sequence
  Advanced Macromolecule Transduction Domain (aMTD) Sequences for Improvement of Cell-Permeability
AVIAVAPLVVAP

SEQ ID NO: 235 - protein, 12 aa, Artificial Sequence
  Advanced Macromolecule Transduction Domain (aMTD) Sequences for Improvement of Cell-Permeability
AVIALAPVVVAP

SEQ ID NO: 236 - protein, 12 aa, Artificial Sequence
  Advanced Macromolecule Transduction Domain (aMTD) Sequences for Improvement of Cell-Permeability
VAIALAPVVVAP

SEQ ID NO: 237 - protein, 12 aa, Artificial Sequence
  Advanced Macromolecule Transduction Domain (aMTD) Sequences for Improvement of Cell-Permeability
VALALAPVVVAP

SEQ ID NO: 238 - protein, 12 aa, Artificial Sequence
  Advanced Macromolecule Transduction Domain (aMTD) Sequences for Improvement of Cell-Permeability
VAALLPAVVVAP

SEQ ID NO: 239 - protein, 12 aa, Artificial Sequence
  Advanced Macromolecule Transduction Domain (aMTD) Sequences for Improvement of Cell-Permeability
VALALPAVVVAP

SEQ ID NO: 240 - protein, 12 aa, Artificial Sequence
  Advanced Macromolecule Transduction Domain (aMTD) Sequences for Improvement of Cell-Permeability
VALLAPAVVVAP

SEQ ID NO: 241 - DNA, 36 bp, Artificial Sequence
  The polynucleotide sequence that encodes advanced Macromolecule Transduction Domain (aMTD) peptide for Improvement of Cell-Permeability
gcggcggcgctggcgccggtggtgctggcgctgccg

SEQ ID NO: 242 - DNA, 36 bp, Artificial Sequence
  The polynucleotide sequence that encodes advanced Macromolecule Transduction Domain (aMTD) peptide for Improvement of Cell-Permeability
gcggcggcggtgccgctgctggcggtggtggtgccg

SEQ ID NO: 243 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcggcggtgctggcgccg

SEQ ID NO: 244 - DNA, 36 bp, Artificial Sequence
  The polynucleotide sequence that encodes advanced Macromolecule Transduction Domain (aMTD) peptide for Improvement of Cell-Permeability
gcgctggcgctgctgccggtggcggcgctggcgccg

SEQ ID NO: 245 - DNA, 36 bp, Artificial Sequence
  The polynucleotide sequence that encodes advanced Macromolecule Transduction Domain (aMTD) peptide for Improvement of Cell-Permeability
gcggcggcgctgctgccggtggcgctggtggcgccg

SEQ ID NO: 246 - DNA, 36 bp, Artificial Sequence
  The polynucleotide sequence that encodes advanced Macromolecule Transduction Domain (aMTD) peptide for Improvement of Cell-Permeability
gtggtggcgctggcgccggcgctggcggcgctgccg

SEQ ID NO: 247 - DNA, 36 bp, Artificial Sequence
  The polynucleotide sequence that encodes advanced Macromolecule Transduction Domain (aMTD) peptide for Improvement of Cell-Permeability
ctgctggcggcggtgccggcggtgctgctggcgccg

SEQ ID NO: 248 - DNA, 36 bp, Artificial Sequence
  The polynucleotide sequence that encodes advanced Macromolecule Transduction Domain (aMTD) peptide for Improvement of Cell-Permeability
gcggcggcgctggtgccggtggtggcgctgctgccg

SEQ ID NO: 249 - DNA, 36 bp, Artificial Sequence
  The polynucleotide sequence that encodes advanced Macromolecule Transduction Domain (aMTD) peptide for Improvement of Cell-Permeability
gcggtggcgctgctgccggcgctgctggcggtgccg

SEQ ID NO: 250 - DNA, 36 bp, Artificial Sequence
  The polynucleotide sequence that encodes advanced Macromolecule Transduction Domain (aMTD) peptide for Improvement of Cell-Permeability
gcggtggtgctggtgccggtgctggcggcggcgccg

SEQ ID NO: 251 - DNA, 36 bp, Artificial Sequence
  The polynucleotide sequence that encodes advanced Macromolecule Transduction Domain (aMTD) peptide for Improvement of Cell-Permeability
gtggtgctggtgctgccggcggcggcggcggtgccg

SEQ ID NO: 252 - DNA, 36 bp, Artificial Sequence
  The polynucleotide sequence that encodes advanced Macromolecule Transduction Domain (aMTD) peptide for Improvement of Cell-Permeability
attgcgctggcggcgccggcgctgattgtggcgccg

SEQ ID NO: 253 - DNA, 36 bp, Artificial Sequence
  The polynucleotide sequence that encodes advanced Macromolecule Transduction Domain (aMTD) peptide for Improvement of Cell-Permeability
attgtggcggtggcgccggcgctggtggcgctgccg

SEQ ID NO: 254 - DNA, 36 bp, Artificial Sequence
  The polynucleotide sequence that encodes advanced Macromolecule Transduction Domain (aMTD) peptide for Improvement of Cell-Permeability
gtggcggcgctgccggtggtggcggtggtggcgccg

SEQ ID NO: 255 - DNA, 36 bp, Artificial Sequence
  The polynucleotide sequence that encodes advanced Macromolecule Transduction Domain (aMTD) peptide for Improvement of Cell-Permeability
ctgctggcggcgccgctggtggtggcggcggtgccg

SEQ ID NO: 256 - DNA, 36 bp, Artificial Sequence
  The polynucleotide sequence that encodes advanced Macromolecule Transduction Domain (aMTD) peptide for Improvement of Cell-Permeability
gcgctggcggtgccggtggcgctgctggtggcgccg

SEQ ID NO: 257 - DNA, 36 bp, Artificial Sequence
  The polynucleotide sequence that encodes advanced Macromolecule Transduction Domain (aMTD) peptide for Improvement of Cell-Permeability
gtggcggcgctgccggtgctgctggcggcgctgccg

SEQ ID NO: 258 - DNA, 36 bp, Artificial Sequence
  The polynucleotide sequence that encodes advanced Macromolecule Transduction Domain (aMTD) peptide for Improvement of Cell-Permeability
gtggcgctgctggcgccggtggcgctggcggtgccg

SEQ ID NO: 259 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcgctggtggcggtgccg

SEQ ID NO: 260 - DNA, 36 bp, Artificial Sequence
  The polynucleotide sequence that encodes advanced Macromolecule Transduction Domain (aMTD) peptide for Improvement of Cell-Permeability
gcgattgtggcgctgccggtggcggtgctggcgccg

SEQ ID NO: 261 - DNA, 36 bp, Artificial Sequence
  The polynucleotide sequence that encodes advanced Macromolecule Transduction Domain (aMTD) peptide for Improvement of Cell-Permeability
attgcgattgtggcgccggtggtggcgctggcgccg

SEQ ID NO: 262 - DNA, 36 bp, Artificial Sequence
  The polynucleotide sequence that encodes advanced Macromolecule Transduction Domain (aMTD) peptide for Improvement of Cell-Permeability
gcggcgctgctgccggcgctggcggcgctgctgccg

SEQ ID NO: 263 - DNA, 36 bp, Artificial Sequence
  The polynucleotide sequence that encodes advanced Macromolecule Transduction Domain (aMTD) peptide for Improvement of Cell-Permeability
gcggtggtgctggcgccggtggcggcggtgctgccg

SEQ ID NO: 264 - DNA, 36 bp, Artificial Sequence
  The polynucleotide sequence that encodes advanced Macromolecule Transduction Domain (aMTD) peptide for Improvement of Cell-Permeability
ctggcggtggcggcgccgctggcgctggcgctgccg

SEQ ID NO: 265 - DNA, 36 bp, Artificial Sequence
  The polynucleotide sequence that encodes advanced Macromolecule Transduction Domain (aMTD) peptide for Improvement of Cell-Permeability
gcggcggtggcggcgccgctgctgctggcgctgccg

SEQ ID NO: 266 - DNA, 36 bp, Artificial Sequence
  The polynucleotide sequence that encodes advanced Macromolecule Transduction Domain (aMTD) peptide for Improvement of Cell-Permeability
ctgctggtgctgccggcggcggcgctggcggcgccg

SEQ ID NO: 267 - DNA, 36 bp, Artificial Sequence
  The polynucleotide sequence that encodes advanced Macromolecule Transduction Domain (aMTD) peptide for Improvement of Cell-Permeability
ctggtggcggtggcgccggtggcggcggtgctgccg

SEQ ID NO: 268 - DNA, 36 bp, Artificial Sequence
  The polynucleotide sequence that encodes advanced Macromolecule Transduction Domain (aMTD) peptide for Improvement of Cell-Permeability
ctggcgctggcgccggcggcgctggcgctgctgccg

SEQ ID NO: 269 - DNA, 36 bp, Artificial Sequence
  The polynucleotide sequence that encodes advanced Macromolecule Transduction Domain (aMTD) peptide for Improvement of Cell-Permeability
gcgctgattgcggcgccgattctggcgctggcgccg

SEQ ID NO: 270 - DNA, 36 bp, Artificial Sequence
  The polynucleotide sequence that encodes advanced Macromolecule Transduction Domain (aMTD) peptide for Improvement of Cell-Permeability
gcggtggtggcggcgccgctggtgctggcgctgccg

SEQ ID NO: 271 - DNA, 36 bp, Artificial Sequence
  The polynucleotide sequence that encodes advanced Macromolecule Transduction Domain (aMTD) peptide for Improvement of Cell-Permeability
ctgctggcgctggcgccggcggcgctgctggcgccg

SEQ ID NO: 272 - DNA, 36 bp, Artificial Sequence
  The polynucleotide sequence that encodes advanced Macromolecule Transduction Domain (aMTD) peptide for Improvement of Cell-Permeability
gcgattgtggcgctgccggcgctggcgctggcgccg

SEQ ID NO: 273 - DNA, 36 bp, Artificial Sequence
  The polynucleotide sequence that encodes advanced Macromolecule Transduction Domain (aMTD) peptide for Improvement of Cell-Permeability
gcggcgattattgtgccggcggcgctgctggcgccg

SEQ ID NO: 274 - DNA, 36 bp, Artificial Sequence
  The polynucleotide sequence that encodes advanced Macromolecule Transduction Domain (aMTD) peptide for Improvement of Cell-Permeability
attgcggtggcgctgccggcgctgattgcggcgccg

SEQ ID NO: 275 - DNA, 36 bp, Artificial Sequence
  The polynucleotide sequence that encodes advanced Macromolecule Transduction Domain (aMTD) peptide for Improvement of Cell-Permeability
gcggtgattgtgctgccggcgctggcggtggcgccg

SEQ ID NO: 276 - DNA, 36 bp, Artificial Sequence
  The polynucleotide sequence that encodes advanced Macromolecule Transduction Domain (aMTD) peptide for Improvement of Cell-Permeability
gcggtgctggcggtgccggcggtgctggtggcgccg

SEQ ID NO: 277 - DNA, 36 bp, Artificial Sequence
  The polynucleotide sequence that encodes advanced Macromolecule Transduction Domain (aMTD) peptide for Improvement of Cell-Permeability
gtgctggcgattgtgccggcggtggcgctggcgccg

SEQ ID NO: 278 - DNA, 36 bp, Artificial Sequence
  The polynucleotide sequence that encodes advanced Macromolecule Transduction Domain (aMTD) peptide for Improvement of Cell-Permeability
ctgctggcggtggtgccggcggtggcgctggcgccg

SEQ ID NO: 279 - DNA, 36 bp, Artificial Sequence
  The polynucleotide sequence that encodes advanced Macromolecule Transduction Domain (aMTD) peptide for Improvement of Cell-Permeability
gcggtgattgcgctgccggcgctgattgcggcgccg

SEQ ID NO: 280 - DNA, 36 bp, Artificial Sequence
  The polynucleotide sequence that encodes advanced Macromolecule Transduction Domain (aMTD) peptide for Improvement of Cell-Permeability
gcggtggtggcgctgccggcggcgctgattgtgccg

SEQ ID NO: 281 - DNA, 36 bp, Artificial Sequence
  The polynucleotide sequence that encodes advanced Macromolecule Transduction Domain (aMTD) peptide for Improvement of Cell-Permeability
ctggcgctggtgctgccggcggcgctggcggcgccg

SEQ ID NO: 282 - DNA, 36 bp, Artificial Sequence
  The polynucleotide sequence that encodes advanced Macromolecule Transduction Domain (aMTD) peptide for Improvement of Cell-Permeability
ctggcggcggtgctgccggcgctgctggcggcgccg

SEQ ID NO: 283 - DNA, 36 bp, Artificial Sequence
  The polynucleotide sequence that encodes advanced Macromolecule Transduction Domain (aMTD) peptide for Improvement of Cell-Permeability
gcgctggcggtgccggtggcgctggcgattgtgccg

SEQ ID NO: 284 - DNA, 36 bp, Artificial Sequence
  The polynucleotide sequence that encodes advanced Macromolecule Transduction Domain (aMTD) peptide for Improvement of Cell-Permeability
gcgctgattgcgccggtggtggcgctggtggcgccg

SEQ ID NO: 285 - DNA, 36 bp, Artificial Sequence
  The polynucleotide sequence that encodes advanced Macromolecule Transduction Domain (aMTD) peptide for Improvement of Cell-Permeability
ctgctggcggcgccggtggtgattgcgctggcgccg

SEQ ID NO: 286 - DNA, 36 bp, Artificial Sequence
  The polynucleotide sequence that encodes advanced Macromolecule Transduction Domain (aMTD) peptide for Improvement of Cell-Permeability
ctggcggcgattgtgccggcgattattgcggtgccg

SEQ ID NO: 287 - DNA, 36 bp, Artificial Sequence
  The polynucleotide sequence that encodes advanced Macromolecule Transduction Domain (aMTD) peptide for Improvement of Cell-Permeability
gcggcgctggtgctgccgctgattattgcggcgccg

SEQ ID NO: 288 - DNA, 36 bp, Artificial Sequence
  The polynucleotide sequence that encodes advanced Macromolecule Transduction Domain (aMTD) peptide for Improvement of Cell-Permeability
ctggcgctggcggtgccggcgctggcggcgctgccg

SEQ ID NO: 289 - DNA, 36 bp, Artificial Sequence
  The polynucleotide sequence that encodes advanced Macromolecule Transduction Domain (aMTD) peptide for Improvement of Cell-Permeability
ctgattgcggcgctgccggcggtggcggcgctgccg

SEQ ID NO: 290 - DNA, 36 bp, Artificial Sequence
  The polynucleotide sequence that encodes advanced Macromolecule Transduction Domain (aMTD) peptide for Improvement of Cell-Permeability
gcgctggcgctggtgccggcgattgcggcgctgccg

SEQ ID NO: 291 - DNA, 36 bp, Artificial Sequence
  The polynucleotide sequence that encodes advanced Macromolecule Transduction Domain (aMTD) peptide for Improvement of Cell-Permeability
gcggcgattctggcgccgattgtggcgctggcgccg

SEQ ID NO: 292 - DNA, 36 bp, Artificial Sequence
  The polynucleotide sequence that encodes advanced Macromolecule Transduction Domain (aMTD) peptide for Improvement of Cell-Permeability
gcgctgctgattgcgccggcggcggtgattgcgccg

SEQ ID NO: 293 - DNA, 36 bp, Artificial Sequence
  The polynucleotide sequence that encodes advanced Macromolecule Transduction Domain (aMTD) peptide for Improvement of Cell-Permeability
gcgattctggcggtgccgattgcggtggtggcgccg

SEQ ID NO: 294 - DNA, 36 bp, Artificial Sequence
  The polynucleotide sequence that encodes advanced Macromolecule Transduction Domain (aMTD) peptide for Improvement of Cell-Permeability
attctggcggcggtgccgattgcgctggcggcgccg

SEQ ID NO: 295 - DNA, 36 bp, Artificial Sequence
  The polynucleotide sequence that encodes advanced Macromolecule Transduction Domain (aMTD) peptide for Improvement of Cell-Permeability
gtggcggcgctgctgccggcggcggcggtgctgccg

SEQ ID NO: 296 - DNA, 36 bp, Artificial Sequence
  The polynucleotide sequence that encodes advanced Macromolecule Transduction Domain (aMTD) peptide for Improvement of Cell-Permeability
gcggcggcggtggtgccggtgctgctggtggcgccg

SEQ ID NO: 297 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcgctggtggcggcgccg

SEQ ID NO: 298 - DNA, 36 bp, Artificial Sequence
  The polynucleotide sequence that encodes advanced Macromolecule Transduction Domain (aMTD) peptide for Improvement of Cell-Permeability
gcggcggtgctgctgccggtggcgctggcggcgccg

SEQ ID NO: 299 - DNA, 36 bp, Artificial Sequence
  The polynucleotide sequence that encodes advanced Macromolecule Transduction Domain (aMTD) peptide for Improvement of Cell-Permeability
gcggcggcgctggcgccggtgctggcgctggtgccg

SEQ ID NO: 300 - DNA, 36 bp, Artificial Sequence
  The polynucleotide sequence that encodes advanced Macromolecule Transduction Domain (aMTD) peptide for Improvement of Cell-Permeability
ctggtgctggtgccgctgctggcggcggcggcgccg

SEQ ID NO: 301 - DNA, 36 bp, Artificial Sequence
  The polynucleotide sequence that encodes advanced Macromolecule Transduction Domain (aMTD) peptide for Improvement of Cell-Permeability
gcgctgattgcggtgccggcgattattgtggcgccg

SEQ ID NO: 302 - DNA, 36 bp, Artificial Sequence
  The polynucleotide sequence that encodes advanced Macromolecule Transduction Domain (aMTD) peptide for Improvement of Cell-Permeability
gcgctggcggtgattccggcggcggcgattctgccg

SEQ ID NO: 303 - DNA, 36 bp, Artificial Sequence
  The polynucleotide sequence that encodes advanced Macromolecule Transduction Domain (aMTD) peptide for Improvement of Cell-Permeability
ctggcggcggcgccggtggtgattgtgattgcgccg

SEQ ID NO: 304 - DNA, 36 bp, Artificial Sequence
  The polynucleotide sequence that encodes advanced Macromolecule Transduction Domain (aMTD) peptide for Improvement of Cell-Permeability
gtgctggcgattgcgccgctgctggcggcggtgccg

SEQ ID NO: 305 - DNA, 36 bp, Artificial Sequence
  The polynucleotide sequence that encodes advanced Macromolecule Transduction Domain (aMTD) peptide for Improvement of Cell-Permeability
gcgctgattgtgctgccggcggcggtggcggtgccg

SEQ ID NO: 306 - DNA, 36 bp, Artificial Sequence
  The polynucleotide sequence that encodes advanced Macromolecule Transduction Domain (aMTD) peptide for Improvement of Cell-Permeability
gtgctggcggtggcgccggcgctgattgtggcgccg

SEQ ID NO: 307 - DNA, 36 bp, Artificial Sequence
  The polynucleotide sequence that encodes advanced Macromolecule Transduction Domain (aMTD) peptide for Improvement of Cell-Permeability
gcggcgctgctggcgccggcgctgattgtggcgccg

SEQ ID NO: 308 - DNA, 36 bp, Artificial Sequence
  The polynucleotide sequence that encodes advanced Macromolecule Transduction Domain (aMTD) peptide for Improvement of Cell-Permeability
gcgctgattgcgccggcggtggcgctgattgtgccg

SEQ ID NO: 309 - DNA, 36 bp, Artificial Sequence
  The polynucleotide sequence that encodes advanced Macromolecule Transduction Domain (aMTD) peptide for Improvement of Cell-Permeability
gcgattgtgctgctgccggcggcggtggtggcgccg

SEQ ID NO: 310 - DNA, 36 bp, Artificial Sequence
  The polynucleotide sequence that encodes advanced Macromolecule Transduction Domain (aMTD) peptide for Improvement of Cell-Permeability
gtgattgcggcgccggtgctggcggtgctggcgccg

SEQ ID NO: 311 - DNA, 36 bp, Artificial Sequence
  The polynucleotide sequence that encodes advanced Macromolecule Transduction Domain (aMTD) peptide for Improvement of Cell-Permeability
ctggcgctggcgccggcgctggcgctgctggcgccg

SEQ ID NO: 312 - DNA, 36 bp, Artificial Sequence
  The polynucleotide sequence that encodes advanced Macromolecule Transduction Domain (aMTD) peptide for Improvement of Cell-Permeability
gcgattattctggcgccgattgcggcgattgcgccg

SEQ ID NO: 313 - DNA, 36 bp, Artificial Sequence
  The polynucleotide sequence that encodes advanced Macromolecule Transduction Domain (aMTD) peptide for Improvement of Cell-Permeability
attgcgctggcggcgccgattctgctggcggcgccg

SEQ ID NO: 314 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcgctggcggtgccg

SEQ ID NO: 315 - DNA, 36 bp, Artificial Sequence
  The polynucleotide sequence that encodes advanced Macromolecule Transduction Domain (aMTD) peptide for Improvement of Cell-Permeability
gtggtggcgattgtgctgccggcgctggcggcgccg

SEQ ID NO: 316 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggtggcgctggcgccg

SEQ ID NO: 317 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggcgctggtgccg

SEQ ID NO: 318 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggtggcgctgccg

SEQ ID NO: 319 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggtgctggcgccg

SEQ ID NO: 320 - DNA, 36 bp, Artificial Sequence
  The polynucleotide sequence that encodes advanced Macromolecule Transduction Domain (aMTD) peptide for Improvement of Cell-Permeability
gtgattgtggcgctggcgccggcggtgctggcgccg

SEQ ID NO: 321 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcgctggtggcgccg

SEQ ID NO: 322 - DNA, 36 bp, Artificial Sequence
  The polynucleotide sequence that encodes advanced Macromolecule Transduction Domain (aMTD) peptide for Improvement of Cell-Permeability
gcgctgctgattgtggcgccggtggcggtggcgccg

SEQ ID NO: 323 - DNA, 36 bp, Artificial Sequence
  The polynucleotide sequence that encodes advanced Macromolecule Transduction Domain (aMTD) peptide for Improvement of Cell-Permeability
gcggtggtgattgtggcgccggcggtgattgcgccg

SEQ ID NO: 324 - DNA, 36 bp, Artificial Sequence
  The polynucleotide sequence that encodes advanced Macromolecule Transduction Domain (aMTD) peptide for Improvement of Cell-Permeability
gcggtgctggcggtggcgccggcgctgattgtgccg

SEQ ID NO: 325 - DNA, 36 bp, Artificial Sequence
  The polynucleotide sequence that encodes advanced Macromolecule Transduction Domain (aMTD) peptide for Improvement of Cell-Permeability
ctggtggcggcggtggcgccggcgctgattgtgccg

SEQ ID NO: 326 - DNA, 36 bp, Artificial Sequence
  The polynucleotide sequence that encodes advanced Macromolecule Transduction Domain (aMTD) peptide for Improvement of Cell-Permeability
gcggtgattgtggtggcgccggcgctgctggcgccg

SEQ ID NO: 327 - DNA, 36 bp, Artificial Sequence
  The polynucleotide sequence that encodes advanced Macromolecule Transduction Domain (aMTD) peptide for Improvement of Cell-Permeability
gtggtggcgattgtgctgccggcggtggcggcgccg

SEQ ID NO: 328 - DNA, 36 bp, Artificial Sequence
  The polynucleotide sequence that encodes advanced Macromolecule Transduction Domain (aMTD) peptide for Improvement of Cell-Permeability
gcggcggcgctggtgattccggcgattctggcgccg

SEQ ID NO: 329 - DNA, 36 bp, Artificial Sequence
  The polynucleotide sequence that encodes advanced Macromolecule Transduction Domain (aMTD) peptide for Improvement of Cell-Permeability
gtgattgtggcgctggcgccggcgctgctggcgccg

SEQ ID NO: 330 - DNA, 36 bp, Artificial Sequence
  The polynucleotide sequence that encodes advanced Macromolecule Transduction Domain (aMTD) peptide for Improvement of Cell-Permeability
gtgattgtggcgattgcgccggcgctgctggcgccg

SEQ ID NO: 331 - DNA, 36 bp, Artificial Sequence
  The polynucleotide sequence that encodes advanced Macromolecule Transduction Domain (aMTD) peptide for Improvement of Cell-Permeability
attgtggcgattgcggtgccggcgctggtggcgccg

SEQ ID NO: 332 - DNA, 36 bp, Artificial Sequence
  The polynucleotide sequence that encodes advanced Macromolecule Transduction Domain (aMTD) peptide for Improvement of Cell-Permeability
gcggcgctggcggtgattccggcggcgattctgccg

SEQ ID NO: 333 - DNA, 36 bp, Artificial Sequence
  The polynucleotide sequence that encodes advanced Macromolecule Transduction Domain (aMTD) peptide for Improvement of Cell-Permeability
gcgctggcggcggtgattccggcggcgattctgccg

SEQ ID NO: 334 - DNA, 36 bp, Artificial Sequence
  The polynucleotide sequence that encodes advanced Macromolecule Transduction Domain (aMTD) peptide for Improvement of Cell-Permeability
gcggcggcgctggtgattccggcggcgattctgccg

SEQ ID NO: 335 - DNA, 36 bp, Artificial Sequence
  The polynucleotide sequence that encodes advanced Macromolecule Transduction Domain (aMTD) peptide for Improvement of Cell-Permeability
ctggcggcggcggtgattccggcggcgattctgccg

SEQ ID NO: 336 - DNA, 36 bp, Artificial Sequence
  The polynucleotide sequence that encodes advanced Macromolecule Transduction Domain (aMTD) peptide for Improvement of Cell-Permeability
ctggcggcggcggtgattccggtggcgattctgccg

SEQ ID NO: 337 - DNA, 36 bp, Artificial Sequence
  The polynucleotide sequence that encodes advanced Macromolecule Transduction Domain (aMTD) peptide for Improvement of Cell-Permeability
gcggcgattctggcggcgccgctgattgcggtgccg

SEQ ID NO: 338 - DNA, 36 bp, Artificial Sequence
  The polynucleotide sequence that encodes advanced Macromolecule Transduction Domain (aMTD) peptide for Improvement of Cell-Permeability
gtggtggcgattctggcgccgctgctggcggcgccg

SEQ ID NO: 339 - DNA, 36 bp, Artificial Sequence
  The polynucleotide sequence that encodes advanced Macromolecule Transduction Domain (aMTD) peptide for Improvement of Cell-Permeability
gcggtggtggtggcggcgccggtgctggcgctgccg

SEQ ID NO: 340 - DNA, 36 bp, Artificial Sequence
  The polynucleotide sequence that encodes advanced Macromolecule Transduction Domain (aMTD) peptide for Improvement of Cell-Permeability
gcggtggtggcgattgcgccggtgctggcgctgccg

SEQ ID NO: 341 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggcggtgctggtgccg

SEQ ID NO: 342 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggtggcgctggtgccg

SEQ ID NO: 343 - DNA, 36 bp, Artificial Sequence
  The polynucleotide sequence that encodes advanced Macromolecule Transduction Domain (aMTD) peptide for Improvement of Cell-Permeability
ctggcggcggcgctggtgccggtggcgctggtgccg

SEQ ID NO: 344 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggcgctggtggtgccg

SEQ ID NO: 345 - DNA, 36 bp, Artificial Sequence
  The polynucleotide sequence that encodes advanced Macromolecule Transduction Domain (aMTD) peptide for Improvement of Cell-Permeability
attgcggcggtgattgtgccggcggtggcgctgccg

SEQ ID NO: 346 - DNA, 36 bp, Artificial Sequence
  The polynucleotide sequence that encodes advanced Macromolecule Transduction Domain (aMTD) peptide for Improvement of Cell-Permeability
attgcggcggtgctggtgccggcggtggcgctgccg

SEQ ID NO: 347 - DNA, 36 bp, Artificial Sequence
  The polynucleotide sequence that encodes advanced Macromolecule Transduction Domain (aMTD) peptide for Improvement of Cell-Permeability
gcggtggcgattctggtgccgctgctggcggcgccg

SEQ ID NO: 348 - DNA, 36 bp, Artificial Sequence
  The polynucleotide sequence that encodes advanced Macromolecule Transduction Domain (aMTD) peptide for Improvement of Cell-Permeability
gcggtggtgattctggtgccgctggcggcggcgccg

SEQ ID NO: 349 - DNA, 36 bp, Artificial Sequence
  The polynucleotide sequence that encodes advanced Macromolecule Transduction Domain (aMTD) peptide for Improvement of Cell-Permeability
attgcggcggtgattgtgccggtggcggcgctgccg

SEQ ID NO: 350 - DNA, 36 bp, Artificial Sequence
  The polynucleotide sequence that encodes advanced Macromolecule Transduction Domain (aMTD) peptide for Improvement of Cell-Permeability
gcgattgcgattgcgattgtgccggtggcgctgccg

SEQ ID NO: 351 - DNA, 36 bp, Artificial Sequence
  The polynucleotide sequence that encodes advanced Macromolecule Transduction Domain (aMTD) peptide for Improvement of Cell-Permeability
attctggcggtggcggcgattccggtggcggtgccg

SEQ ID NO: 352 - DNA, 36 bp, Artificial Sequence
  The polynucleotide sequence that encodes advanced Macromolecule Transduction Domain (aMTD) peptide for Improvement of Cell-Permeability
attctggcggcggcgattattccggcggcgctgccg

SEQ ID NO: 353 - DNA, 36 bp, Artificial Sequence
  The polynucleotide sequence that encodes advanced Macromolecule Transduction Domain (aMTD) peptide for Improvement of Cell-Permeability
ctggcggtggtgctggcggcgccggcgattgtgccg

SEQ ID NO: 354 - DNA, 36 bp, Artificial Sequence
  The polynucleotide sequence that encodes advanced Macromolecule Transduction Domain (aMTD) peptide for Improvement of Cell-Permeability
gcgattctggcggcgattgtgccgctggcggtgccg

SEQ ID NO: 355 - DNA, 36 bp, Artificial Sequence
  The polynucleotide sequence that encodes advanced Macromolecule Transduction Domain (aMTD) peptide for Improvement of Cell-Permeability
gtgattgtggcgctggcggtgccggcgctggcgccg

SEQ ID NO: 356 - DNA, 36 bp, Artificial Sequence
  The polynucleotide sequence that encodes advanced Macromolecule Transduction Domain (aMTD) peptide for Improvement of Cell-Permeability
gcgattgtggcgctggcggtgccggtgctggcgccg

SEQ ID NO: 357 - DNA, 36 bp, Artificial Sequence
  The polynucleotide sequence that encodes advanced Macromolecule Transduction Domain (aMTD) peptide for Improvement of Cell-Permeability
gcggcgattattattgtgctgccggcggcgctgccg

SEQ ID NO: 358 - DNA, 36 bp, Artificial Sequence
  The polynucleotide sequence that encodes advanced Macromolecule Transduction Domain (aMTD) peptide for Improvement of Cell-Permeability
ctgattgtggcgctggcggtgccggcgctggcgccg

SEQ ID NO: 359 - DNA, 36 bp, Artificial Sequence
  The polynucleotide sequence that encodes advanced Macromolecule Transduction Domain (aMTD) peptide for Improvement of Cell-Permeability
gcgattattattgtgattgcgccggcggcggcgccg

SEQ ID NO: 360 - DNA, 36 bp, Artificial Sequence
  The polynucleotide sequence that encodes advanced Macromolecule Transduction Domain (aMTD) peptide for Improvement of Cell-Permeability
ctggcggcgctgattgtggtgccggcggtggcgccg

SEQ ID NO: 361 - DNA, 36 bp, Artificial Sequence
  The polynucleotide sequence that encodes advanced Macromolecule Transduction Domain (aMTD) peptide for Improvement of Cell-Permeability
gcgctgctggtgattgcggtgccggcggtggcgccg

SEQ ID NO: 362 - DNA, 36 bp, Artificial Sequence
  The polynucleotide sequence that encodes advanced Macromolecule Transduction Domain (aMTD) peptide for Improvement of Cell-Permeability
gcggtggcgctgattgtggtgccggcgctggcgccg

SEQ ID NO: 363 - DNA, 36 bp, Artificial Sequence
  The polynucleotide sequence that encodes advanced Macromolecule Transduction Domain (aMTD) peptide for Improvement of Cell-Permeability
gcgctggcgattgtggtggcgccggtggcggtgccg

SEQ ID NO: 364 - DNA, 36 bp, Artificial Sequence
  The polynucleotide sequence that encodes advanced Macromolecule Transduction Domain (aMTD) peptide for Improvement of Cell-Permeability
ctgctggcgctgattattgcgccggcggcggcgccg

SEQ ID NO: 365 - DNA, 36 bp, Artificial Sequence
  The polynucleotide sequence that encodes advanced Macromolecule Transduction Domain (aMTD) peptide for Improvement of Cell-Permeability
gcgctggcgctgattattgtgccggcggtggcgccg

SEQ ID NO: 366 - DNA, 36 bp, Artificial Sequence
  The polynucleotide sequence that encodes advanced Macromolecule Transduction Domain (aMTD) peptide for Improvement of Cell-Permeability
ctgctggcggcgctgattgcgccggcggcgctgccg

SEQ ID NO: 367 - DNA, 36 bp, Artificial Sequence
  The polynucleotide sequence that encodes advanced Macromolecule Transduction Domain (aMTD) peptide for Improvement of Cell-Permeability
attgtggcgctgattgtggcgccggcggcggtgccg

SEQ ID NO: 368 - DNA, 36 bp, Artificial Sequence
  The polynucleotide sequence that encodes advanced Macromolecule Transduction Domain (aMTD) peptide for Improvement of Cell-Permeability
gtggtgctggtgctggcggcgccggcggcggtgccg

SEQ ID NO: 369 - DNA, 36 bp, Artificial Sequence
  The polynucleotide sequence that encodes advanced Macromolecule Transduction Domain (aMTD) peptide for Improvement of Cell-Permeability
gcggcggtggcgattgtgctgccggcggtggtgccg

SEQ ID NO: 370 - DNA, 36 bp, Artificial Sequence
  The polynucleotide sequence that encodes advanced Macromolecule Transduction Domain (aMTD) peptide for Improvement of Cell-Permeability
gcgctgattgcggcgattgtgccggcgctggtgccg

SEQ ID NO: 371 - DNA, 36 bp, Artificial Sequence
  The polynucleotide sequence that encodes advanced Macromolecule Transduction Domain (aMTD) peptide for Improvement of Cell-Permeability
gcgctggcggtgattgtggtgccggcgctggcgccg

SEQ ID NO: 372 - DNA, 36 bp, Artificial Sequence
  The polynucleotide sequence that encodes advanced Macromolecule Transduction Domain (aMTD) peptide for Improvement of Cell-Permeability
gtggcgattgcgctgattgtgccggcgctggcgccg

SEQ ID NO: 373 - DNA, 36 bp, Artificial Sequence
  The polynucleotide sequence that encodes advanced Macromolecule Transduction Domain (aMTD) peptide for Improvement of Cell-Permeability
gtggcgattgtgctggtggcgccggcggtggcgccg

SEQ ID NO: 374 - DNA, 36 bp, Artificial Sequence
  The polynucleotide sequence that encodes advanced Macromolecule Transduction Domain (aMTD) peptide for Improvement of Cell-Permeability
gtggcggtggcgctgattgtgccggcgctggcgccg

SEQ ID NO: 375 - DNA, 36 bp, Artificial Sequence
  The polynucleotide sequence that encodes advanced Macromolecule Transduction Domain (aMTD) peptide for Improvement of Cell-Permeability
gcggtgattctggcgctggcgccgattgtggcgccg

SEQ ID NO: 376 - DNA, 36 bp, Artificial Sequence
  The polynucleotide sequence that encodes advanced Macromolecule Transduction Domain (aMTD) peptide for Improvement of Cell-Permeability
gcgctgattgtggcgattgcgccggcgctggtgccg

SEQ ID NO: 377 - DNA, 36 bp, Artificial Sequence
  The polynucleotide sequence that encodes advanced Macromolecule Transduction Domain (aMTD) peptide for Improvement of Cell-Permeability
gcggcgattctgattgcggtgccgattgcggcgccg

SEQ ID NO: 378 - DNA, 36 bp, Artificial Sequence
  The polynucleotide sequence that encodes advanced Macromolecule Transduction Domain (aMTD) peptide for Improvement of Cell-Permeability
gtgattgtggcgctggcggcgccggtgctggcgccg

SEQ ID NO: 379 - DNA, 36 bp, Artificial Sequence
  The polynucleotide sequence that encodes advanced Macromolecule Transduction Domain (aMTD) peptide for Improvement of Cell-Permeability
gtgctggtggcgctggcggcgccggtgattgcgccg

SEQ ID NO: 380 - DNA, 36 bp, Artificial Sequence
  The polynucleotide sequence that encodes advanced Macromolecule Transduction Domain (aMTD) peptide for Improvement of Cell-Permeability
gtggcgctgattgcggtggcgccggcggtggtgccg

SEQ ID NO: 381 - DNA, 36 bp, Artificial Sequence
  The polynucleotide sequence that encodes advanced Macromolecule Transduction Domain (aMTD) peptide for Improvement of Cell-Permeability
gtgattgcggcggtgctggcgccggtggcggtgccg

SEQ ID NO: 382 - DNA, 36 bp, Artificial Sequence
  The polynucleotide sequence that encodes advanced Macromolecule Transduction Domain (aMTD) peptide for Improvement of Cell-Permeability
gcgctgattgtgctggcggcgccggtggcggtgccg

SEQ ID NO: 383 - DNA, 36 bp, Artificial Sequence
  The polynucleotide sequence that encodes advanced Macromolecule Transduction Domain (aMTD) peptide for Improvement of Cell-Permeability
gtggcggcggcgattgcgctgccggcgattgtgccg

SEQ ID NO: 384 - DNA, 36 bp, Artificial Sequence
  The polynucleotide sequence that encodes advanced Macromolecule Transduction Domain (aMTD) peptide for Improvement of Cell-Permeability
attctggcggcggcggcggcgccgctgattgtgccg

SEQ ID NO: 385 - DNA, 36 bp, Artificial Sequence
  The polynucleotide sequence that encodes advanced Macromolecule Transduction Domain (aMTD) peptide for Improvement of Cell-Permeability
ctggcgctggtgctggcggcgccggcgattgtgccg

SEQ ID NO: 386 - DNA, 36 bp, Artificial Sequence
  The polynucleotide sequence that encodes advanced Macromolecule Transduction Domain (aMTD) peptide for Improvement of Cell-Permeability
gcgctggcggtggtggcgctgccggcgattgtgccg

SEQ ID NO: 387 - DNA, 36 bp, Artificial Sequence
  The polynucleotide sequence that encodes advanced Macromolecule Transduction Domain (aMTD) peptide for Improvement of Cell-Permeability
gcggcgattctggcgccgattgtggcggcgctgccg

SEQ ID NO: 388 - DNA, 36 bp, Artificial Sequence
  The polynucleotide sequence that encodes advanced Macromolecule Transduction Domain (aMTD) peptide for Improvement of Cell-Permeability
attctgattgcgattgcgattccggcggcggcgccg

SEQ ID NO: 389 - DNA, 36 bp, Artificial Sequence
  The polynucleotide sequence that encodes advanced Macromolecule Transduction Domain (aMTD) peptide for Improvement of Cell-Permeability
ctggcgattgtgctggcggcgccggtggcggtgccg

SEQ ID NO: 390 - DNA, 36 bp, Artificial Sequence
  The polynucleotide sequence that encodes advanced Macromolecule Transduction Domain (aMTD) peptide for Improvement of Cell-Permeability
gcggcgattgcgattattgcgccggcgattgtgccg

SEQ ID NO: 391 - DNA, 36 bp, Artificial Sequence
  The polynucleotide sequence that encodes advanced Macromolecule Transduction Domain (aMTD) peptide for Improvement of Cell-Permeability
ctggcggtggcgattgtggcgccggcgctggtgccg

SEQ ID NO: 392 - DNA, 36 bp, Artificial Sequence
  The polynucleotide sequence that encodes advanced Macromolecule Transduction Domain (aMTD) peptide for Improvement of Cell-Permeability
ctggcgattgtgctggcggcgccggcggtgctgccg

SEQ ID NO: 393 - DNA, 36 bp, Artificial Sequence
  The polynucleotide sequence that encodes advanced Macromolecule Transduction Domain (aMTD) peptide for Improvement of Cell-Permeability
gcggcgattgtgctggcgctgccggcggtgctgccg

SEQ ID NO: 394 - DNA, 36 bp, Artificial Sequence
  The polynucleotide sequence that encodes advanced Macromolecule Transduction Domain (aMTD) peptide for Improvement of Cell-Permeability
gcgctgctggtggcggtgctgccggcggcgctgccg

SEQ ID NO: 395 - DNA, 36 bp, Artificial Sequence
  The polynucleotide sequence that encodes advanced Macromolecule Transduction Domain (aMTD) peptide for Improvement of Cell-Permeability
gcggcgctggtggcggtgctgccggtggcgctgccg

SEQ ID NO: 396 - DNA, 36 bp, Artificial Sequence
  The polynucleotide sequence that encodes advanced Macromolecule Transduction Domain (aMTD) peptide for Improvement of Cell-Permeability
gcgattctggcggtggcgctgccgctgctggcgccg

SEQ ID NO: 397 - DNA, 36 bp, Artificial Sequence
  The polynucleotide sequence that encodes advanced Macromolecule Transduction Domain (aMTD) peptide for Improvement of Cell-Permeability
attgtggcggtggcgctggtgccggcgctggcgccg

SEQ ID NO: 398 - DNA, 36 bp, Artificial Sequence
  The polynucleotide sequence that encodes advanced Macromolecule Transduction Domain (aMTD) peptide for Improvement of Cell-Permeability
attgtggcggtggcgctgctgccggcgctggcgccg

SEQ ID NO: 399 - DNA, 36 bp, Artificial Sequence
  The polynucleotide sequence that encodes advanced Macromolecule Transduction Domain (aMTD) peptide for Improvement of Cell-Permeability
attgtggcggtggcgctgctgccggcggtggcgccg

SEQ ID NO: 400 - DNA, 36 bp, Artificial Sequence
  The polynucleotide sequence that encodes advanced Macromolecule Transduction Domain (aMTD) peptide for Improvement of Cell-Permeability
attgtggcgctggcggtgctgccggcggtggcgccg

SEQ ID NO: 401 - DNA, 36 bp, Artificial Sequence
  The polynucleotide sequence that encodes advanced Macromolecule Transduction Domain (aMTD) peptide for Improvement of Cell-Permeability
gtggcggtgctggcggtgctgccggcgctggcgccg

SEQ ID NO: 402 - DNA, 36 bp, Artificial Sequence
  The polynucleotide sequence that encodes advanced Macromolecule Transduction Domain (aMTD) peptide for Improvement of Cell-Permeability
attgcggtgctggcggtggcgccggcggtgctgccg

SEQ ID NO: 403 - DNA, 36 bp, Artificial Sequence
  The polynucleotide sequence that encodes advanced Macromolecule Transduction Domain (aMTD) peptide for Improvement of Cell-Permeability
ctggcggtggcgattattgcgccggcggtggcgccg

SEQ ID NO: 404 - DNA, 36 bp, Artificial Sequence
  The polynucleotide sequence that encodes advanced Macromolecule Transduction Domain (aMTD) peptide for Improvement of Cell-Permeability
gtggcgctggcgattgcgctgccggcggtgctgccg

SEQ ID NO: 405 - DNA, 36 bp, Artificial Sequence
  The polynucleotide sequence that encodes advanced Macromolecule Transduction Domain (aMTD) peptide for Improvement of Cell-Permeability
gcgattgcgattgcgctggtgccggtggcgctgccg

SEQ ID NO: 406 - DNA, 36 bp, Artificial Sequence
  The polynucleotide sequence that encodes advanced Macromolecule Transduction Domain (aMTD) peptide for Improvement of Cell-Permeability
gcggcggtggtgattgtggcgccggtggcgctgccg

SEQ ID NO: 407 - DNA, 36 bp, Artificial Sequence
  The polynucleotide sequence that encodes advanced Macromolecule Transduction Domain (aMTD) peptide for Improvement of Cell-Permeability
gcggcgattctggcgattgtggcgccgctggcgccg

SEQ ID NO: 408 - DNA, 36 bp, Artificial Sequence
  The polynucleotide sequence that encodes advanced Macromolecule Transduction Domain (aMTD) peptide for Improvement of Cell-Permeability
gtggcgctgctggcgattgcgccggcgctggcgccg

SEQ ID NO: 409 - DNA, 36 bp, Artificial Sequence
  The polynucleotide sequence that encodes advanced Macromolecule Transduction Domain (aMTD) peptide for Improvement of Cell-Permeability
gtggcggtgctgattgcggtgccggcgctggcgccg

SEQ ID NO: 410 - DNA, 36 bp, Artificial Sequence
  The polynucleotide sequence that encodes advanced Macromolecule Transduction Domain (aMTD) peptide for Improvement of Cell-Permeability
gcggtggcgctggcggtgctgccggcggtggtgccg

SEQ ID NO: 411 - DNA, 36 bp, Artificial Sequence
  The polynucleotide sequence that encodes advanced Macromolecule Transduction Domain (aMTD) peptide for Improvement of Cell-Permeability
gcggtggcgctggcggtggtgccggcggtgctgccg

SEQ ID NO: 412 - DNA, 36 bp, Artificial Sequence
  The polynucleotide sequence that encodes advanced Macromolecule Transduction Domain (aMTD) peptide for Improvement of Cell-Permeability
attgtggtgattgcggtggcgccggcggtggcgccg

SEQ ID NO: 413 - DNA, 36 bp, Artificial Sequence
  The polynucleotide sequence that encodes advanced Macromolecule Transduction Domain (aMTD) peptide for Improvement of Cell-Permeability
attgtggtggcggcggtggtgccggcgctggcgccg

SEQ ID NO: 414 - DNA, 36 bp, Artificial Sequence
  The polynucleotide sequence that encodes advanced Macromolecule Transduction Domain (aMTD) peptide for Improvement of Cell-Permeability
attgtggcgctggtgccggcggtggcgattgcgccg

SEQ ID NO: 415 - DNA, 36 bp, Artificial Sequence
  The polynucleotide sequence that encodes advanced Macromolecule Transduction Domain (aMTD) peptide for Improvement of Cell-Permeability
gtggcggcgctgccggcggtggcgctggtggtgccg

SEQ ID NO: 416 - DNA, 36 bp, Artificial Sequence
  The polynucleotide sequence that encodes advanced Macromolecule Transduction Domain (aMTD) peptide for Improvement of Cell-Permeability
ctggtggcgattgcgccgctggcggtgctggcgccg

SEQ ID NO: 417 - DNA, 36 bp, Artificial Sequence
  The polynucleotide sequence that encodes advanced Macromolecule Transduction Domain (aMTD) peptide for Improvement of Cell-Permeability
gcggtggcgctggtgccggtgattgtggcggcgccg

SEQ ID NO: 418 - DNA, 36 bp, Artificial Sequence
  The polynucleotide sequence that encodes advanced Macromolecule Transduction Domain (aMTD) peptide for Improvement of Cell-Permeability
gcgattgcggtggcgattgcgccggtggcgctgccg

SEQ ID NO: 419 - DNA, 36 bp, Artificial Sequence
  The polynucleotide sequence that encodes advanced Macromolecule Transduction Domain (aMTD) peptide for Improvement of Cell-Permeability
gcgattgcgctggcggtgccggtgctggcgctgccg

SEQ ID NO: 420 - DNA, 36 bp, Artificial Sequence
  The polynucleotide sequence that encodes advanced Macromolecule Transduction Domain (aMTD) peptide for Improvement of Cell-Permeability
ctggtgctgattgcggcggcgccgattgcgctgccg

SEQ ID NO: 421 - DNA, 36 bp, Artificial Sequence
  The polynucleotide sequence that encodes advanced Macromolecule Transduction Domain (aMTD) peptide for Improvement of Cell-Permeability
ctggtggcgctggcggtgccggcggcggtgctgccg

SEQ ID NO: 422 - DNA, 36 bp, Artificial Sequence
  The polynucleotide sequence that encodes advanced Macromolecule Transduction Domain (aMTD) peptide for Improvement of Cell-Permeability
gcggtggcgctggcggtgccggcgctggtgctgccg

SEQ ID NO: 423 - DNA, 36 bp, Artificial Sequence
  The polynucleotide sequence that encodes advanced Macromolecule Transduction Domain (aMTD) peptide for Improvement of Cell-Permeability
ctggtggtgctggcggcggcgccgctggcggtgccg

SEQ ID NO: 424 - DNA, 36 bp, Artificial Sequence
  The polynucleotide sequence that encodes advanced Macromolecule Transduction Domain (aMTD) peptide for Improvement of Cell-Permeability
ctgattgtgctggcggcgccggcgctggcggcgccg

SEQ ID NO: 425 - DNA, 36 bp, Artificial Sequence
  The polynucleotide sequence that encodes advanced Macromolecule Transduction Domain (aMTD) peptide for Improvement of Cell-Permeability
gtgattgtgctggcggcgccggcgctggcggcgccg

SEQ ID NO: 426 - DNA, 36 bp, Artificial Sequence
  The polynucleotide sequence that encodes advanced Macromolecule Transduction Domain (aMTD) peptide for Improvement of Cell-Permeability
gcggtggtgctggcggtgccggcgctggcggtgccg

SEQ ID NO: 427 - DNA, 36 bp, Artificial Sequence
  The polynucleotide sequence that encodes advanced Macromolecule Transduction Domain (aMTD) peptide for Improvement of Cell-Permeability
ctgattattgtggcggcggcgccggcggtggcgccg

SEQ ID NO: 428 - DNA, 36 bp, Artificial Sequence
  The polynucleotide sequence that encodes advanced Macromolecule Transduction Domain (aMTD) peptide for Improvement of Cell-Permeability
attgtggcggtgattgtggcgccggcggtggcgccg

SEQ ID NO: 429 - DNA, 36 bp, Artificial Sequence
  The polynucleotide sequence that encodes advanced Macromolecule Transduction Domain (aMTD) peptide for Improvement of Cell-Permeability
ctggtggcgctggcggcgccgattattgcggtgccg

SEQ ID NO: 430 - DNA, 36 bp, Artificial Sequence
  The polynucleotide sequence that encodes advanced Macromolecule Transduction Domain (aMTD) peptide for Improvement of Cell-Permeability
attgcggcggtgctggcggcgccggcgctggtgccg

SEQ ID NO: 431 - DNA, 36 bp, Artificial Sequence
  The polynucleotide sequence that encodes advanced Macromolecule Transduction Domain (aMTD) peptide for Improvement of Cell-Permeability
attgcgctgctggcggcgccgattattgcggtgccg

SEQ ID NO: 432 - DNA, 36 bp, Artificial Sequence
  The polynucleotide sequence that encodes advanced Macromolecule Transduction Domain (aMTD) peptide for Improvement of Cell-Permeability
gcggcgctggcgctggtggcgccggtgattgtgccg

SEQ ID NO: 433 - DNA, 36 bp, Artificial Sequence
  The polynucleotide sequence that encodes advanced Macromolecule Transduction Domain (aMTD) peptide for Improvement of Cell-Permeability
attgcgctggtggcggcgccggtggcgctggtgccg

SEQ ID NO: 434 - DNA, 36 bp, Artificial Sequence
  The polynucleotide sequence that encodes advanced Macromolecule Transduction Domain (aMTD) peptide for Improvement of Cell-Permeability
attattgtggcggtggcgccggcggcgattgtgccg

SEQ ID NO: 435 - DNA, 36 bp, Artificial Sequence
  The polynucleotide sequence that encodes advanced Macromolecule Transduction Domain (aMTD) peptide for Improvement of Cell-Permeability
gcggtggcggcgattgtgccggtgattgtggcgccg

SEQ ID NO: 436 - DNA, 36 bp, Artificial Sequence
  The polynucleotide sequence that encodes advanced Macromolecule Transduction Domain (aMTD) peptide for Improvement of Cell-Permeability
gcggtgctggtgctggtggcgccggcggcggcgccg

SEQ ID NO: 437 - DNA, 36 bp, Artificial Sequence
  The polynucleotide sequence that encodes advanced Macromolecule Transduction Domain (aMTD) peptide for Improvement of Cell-Permeability
gtggtggcgctgctggcgccgctgattgcggcgccg

SEQ ID NO: 438 - DNA, 36 bp, Artificial Sequence
  The polynucleotide sequence that encodes advanced Macromolecule Transduction Domain (aMTD) peptide for Improvement of Cell-Permeability
gcggcggtggtgattgcgccgctgctggcggtgccg

SEQ ID NO: 439 - DNA, 36 bp, Artificial Sequence
  The polynucleotide sequence that encodes advanced Macromolecule Transduction Domain (aMTD) peptide for Improvement of Cell-Permeability
attgcggtggcggtggcggcgccgctgctggtgccg

SEQ ID NO: 440 - DNA, 36 bp, Artificial Sequence
  The polynucleotide sequence that encodes advanced Macromolecule Transduction Domain (aMTD) peptide for Improvement of Cell-Permeability
ctggtggcgattgtggtgctgccggcggtggcgccg

SEQ ID NO: 441 - DNA, 36 bp, Artificial Sequence
  The polynucleotide sequence that encodes advanced Macromolecule Transduction Domain (aMTD) peptide for Improvement of Cell-Permeability
gcggtggcgattgtggtgctgccggcggtggcgccg

SEQ ID NO: 442 - DNA, 36 bp, Artificial Sequence
  The polynucleotide sequence that encodes advanced Macromolecule Transduction Domain (aMTD) peptide for Improvement of Cell-Permeability
gcggtgattctgctggcgccgctgattgcggcgccg

SEQ ID NO: 443 - DNA, 36 bp, Artificial Sequence
  The polynucleotide sequence that encodes advanced Macromolecule Transduction Domain (aMTD) peptide for Improvement of Cell-Permeability
ctggtgattgcgctggcggcgccggtggcgctgccg

SEQ ID NO: 444 - DNA, 36 bp, Artificial Sequence
  The polynucleotide sequence that encodes advanced Macromolecule Transduction Domain (aMTD) peptide for Improvement of Cell-Permeability
gtgctggcggtggtgctgccggcggtggcgctgccg

SEQ ID NO: 445 - DNA, 36 bp, Artificial Sequence
  The polynucleotide sequence that encodes advanced Macromolecule Transduction Domain (aMTD) peptide for Improvement of Cell-Permeability
gtgctggcggtggcggcgccggcggtgctgctgccg

SEQ ID NO: 446 - DNA, 36 bp, Artificial Sequence
  The polynucleotide sequence that encodes advanced Macromolecule Transduction Domain (aMTD) peptide for Improvement of Cell-Permeability
gcggcggtggtgctgctgccgattattgcggcgccg

SEQ ID NO: 447 - DNA, 36 bp, Artificial Sequence
  The polynucleotide sequence that encodes advanced Macromolecule Transduction Domain (aMTD) peptide for Improvement of Cell-Permeability
gcgctgctggtgattgcgccggcgattgcggtgccg

SEQ ID NO: 448 - DNA, 36 bp, Artificial Sequence
  The polynucleotide sequence that encodes advanced Macromolecule Transduction Domain (aMTD) peptide for Improvement of Cell-Permeability
gcggtgctggtgattgcggtgccggcgattgcgccg

SEQ ID NO: 449 - DNA, 36 bp, Artificial Sequence
  The polynucleotide sequence that encodes advanced Macromolecule Transduction Domain (aMTD) peptide for Improvement of Cell-Permeability
gcgctgctggtggtgattgcgccgctggcggcgccg

SEQ ID NO: 450 - DNA, 36 bp, Artificial Sequence
  The polynucleotide sequence that encodes advanced Macromolecule Transduction Domain (aMTD) peptide for Improvement of Cell-Permeability
gtgctggtggcggcgattctgccggcggcgattccg

SEQ ID NO: 451 - DNA, 36 bp, Artificial Sequence
  The polynucleotide sequence that encodes advanced Macromolecule Transduction Domain (aMTD) peptide for Improvement of Cell-Permeability
gtgctggtggcggcggtgctgccgattgcggcgccg

SEQ ID NO: 452 - DNA, 36 bp, Artificial Sequence
  The polynucleotide sequence that encodes advanced Macromolecule Transduction Domain (aMTD) peptide for Improvement of Cell-Permeability
gtgctggcggcggcggtgctgccgctggtggtgccg

SEQ ID NO: 453 - DNA, 36 bp, Artificial Sequence
  The polynucleotide sequence that encodes advanced Macromolecule Transduction Domain (aMTD) peptide for Improvement of Cell-Permeability
gcgattgcgattgtggtgccggcggtggcggtgccg

SEQ ID NO: 454 - DNA, 36 bp, Artificial Sequence
  The polynucleotide sequence that encodes advanced Macromolecule Transduction Domain (aMTD) peptide for Improvement of Cell-Permeability
gtggcgattattgcggtgccggcggtggtggcgccg

SEQ ID NO: 455 - DNA, 36 bp, Artificial Sequence
  The polynucleotide sequence that encodes advanced Macromolecule Transduction Domain (aMTD) peptide for Improvement of Cell-Permeability
attgtggcgctggtggcgccggcggcggtggtgccg

SEQ ID NO: 456 - DNA, 36 bp, Artificial Sequence
  The polynucleotide sequence that encodes advanced Macromolecule Transduction Domain (aMTD) peptide for Improvement of Cell-Permeability
gcggcgattgtgctgctgccggcggtggtggtgccg

SEQ ID NO: 457 - DNA, 36 bp, Artificial Sequence
  The polynucleotide sequence that encodes advanced Macromolecule Transduction Domain (aMTD) peptide for Improvement of Cell-Permeability
gcggcgctgattgtggtgccggcggtggcggtgccg

SEQ ID NO: 458 - DNA, 36 bp, Artificial Sequence
  The polynucleotide sequence that encodes advanced Macromolecule Transduction Domain (aMTD) peptide for Improvement of Cell-Permeability
gcgattgcgctggtggtgccggcggtggcggtgccg

SEQ ID NO: 459 - DNA, 36 bp, Artificial Sequence
  The polynucleotide sequence that encodes advanced Macromolecule Transduction Domain (aMTD) peptide for Improvement of Cell-Permeability
ctggcgattgtgccggcggcgattgcggcgctgccg

SEQ ID NO: 460 - DNA, 36 bp, Artificial Sequence
  The polynucleotide sequence that encodes advanced Macromolecule Transduction Domain (aMTD) peptide for Improvement of Cell-Permeability
ctggtggcgattgcgccggcggtggcggtgctgccg

SEQ ID NO: 461 - DNA, 36 bp, Artificial Sequence
  The polynucleotide sequence that encodes advanced Macromolecule Transduction Domain (aMTD) peptide for Improvement of Cell-Permeability
gtgctggcggtggcgccggcggtggcggtgctgccg

SEQ ID NO: 462 - DNA, 36 bp, Artificial Sequence
  The polynucleotide sequence that encodes advanced Macromolecule Transduction Domain (aMTD) peptide for Improvement of Cell-Permeability
attctggcggtggtggcgattccggcggcggcgccg

SEQ ID NO: 463 - DNA, 36 bp, Artificial Sequence
  The polynucleotide sequence that encodes advanced Macromolecule Transduction Domain (aMTD) peptide for Improvement of Cell-Permeability
attctggtggcggcggcgccgattgcggcgctgccg

SEQ ID NO: 464 - DNA, 36 bp, Artificial Sequence
  The polynucleotide sequence that encodes advanced Macromolecule Transduction Domain (aMTD) peptide for Improvement of Cell-Permeability
attctggcggtggcggcgattccggcggcgctgccg

SEQ ID NO: 465 - DNA, 36 bp, Artificial Sequence
  The polynucleotide sequence that encodes advanced Macromolecule Transduction Domain (aMTD) peptide for Improvement of Cell-Permeability
gtgattgcgattccggcgattctggcggcggcgccg

SEQ ID NO: 466 - DNA, 36 bp, Artificial Sequence
  The polynucleotide sequence that encodes advanced Macromolecule Transduction Domain (aMTD) peptide for Improvement of Cell-Permeability
gcgattattattgtggtgccggcgattgcggcgccg

SEQ ID NO: 467 - DNA, 36 bp, Artificial Sequence
  The polynucleotide sequence that encodes advanced Macromolecule Transduction Domain (aMTD) peptide for Improvement of Cell-Permeability
gcgattctgattgtggtggcgccgattgcggcgccg

SEQ ID NO: 468 - DNA, 36 bp, Artificial Sequence
  The polynucleotide sequence that encodes advanced Macromolecule Transduction Domain (aMTD) peptide for Improvement of Cell-Permeability
gcggtgattgtgccggtggcgattattgcggcgccg

SEQ ID NO: 469 - DNA, 36 bp, Artificial Sequence
  The polynucleotide sequence that encodes advanced Macromolecule Transduction Domain (aMTD) peptide for Improvement of Cell-Permeability
gcggtggtgattgcgctgccggcggtggtggcgccg

SEQ ID NO: 470 - DNA, 36 bp, Artificial Sequence
  The polynucleotide sequence that encodes advanced Macromolecule Transduction Domain (aMTD) peptide for Improvement of Cell-Permeability
gcgctggtggcggtgattgcgccggtggtggcgccg

SEQ ID NO: 471 - DNA, 36 bp, Artificial Sequence
  The polynucleotide sequence that encodes advanced Macromolecule Transduction Domain (aMTD) peptide for Improvement of Cell-Permeability
gcgctggtggcggtgctgccggcggtggcggtgccg

SEQ ID NO: 472 - DNA, 36 bp, Artificial Sequence
  The polynucleotide sequence that encodes advanced Macromolecule Transduction Domain (aMTD) peptide for Improvement of Cell-Permeability
gcgctggtggcgccgctgctggcggtggcggtgccg

SEQ ID NO: 473 - DNA, 36 bp, Artificial Sequence
  The polynucleotide sequence that encodes advanced Macromolecule Transduction Domain (aMTD) peptide for Improvement of Cell-Permeability
gcggtgctggcggtggtggcgccggtggtggcgccg

SEQ ID NO: 474 - DNA, 36 bp, Artificial Sequence
  The polynucleotide sequence that encodes advanced Macromolecule Transduction Domain (aMTD) peptide for Improvement of Cell-Permeability
gcggtgattgcggtggcgccgctggtggtggcgccg

SEQ ID NO: 475 - DNA, 36 bp, Artificial Sequence
  The polynucleotide sequence that encodes advanced Macromolecule Transduction Domain (aMTD) peptide for Improvement of Cell-Permeability
gcggtgattgcgctggcgccggtggtggtggcgccg

SEQ ID NO: 476 - DNA, 36 bp, Artificial Sequence
  The polynucleotide sequence that encodes advanced Macromolecule Transduction Domain (aMTD) peptide for Improvement of Cell-Permeability
gtggcgattgcgctggcgccggtggtggtggcgccg

SEQ ID NO: 477 - DNA, 36 bp, Artificial Sequence
  The polynucleotide sequence that encodes advanced Macromolecule Transduction Domain (aMTD) peptide for Improvement of Cell-Permeability
gtggcgctggcgctggcgccggtggtggtggcgccg

SEQ ID NO: 478 - DNA, 36 bp, Artificial Sequence
  The polynucleotide sequence that encodes advanced Macromolecule Transduction Domain (aMTD) peptide for Improvement of Cell-Permeability
gtggcggcgctgctgccggcggtggtggtggcgccg

SEQ ID NO: 479 - DNA, 36 bp, Artificial Sequence
  The polynucleotide sequence that encodes advanced Macromolecule Transduction Domain (aMTD) peptide for Improvement of Cell-Permeability
gtggcgctggcgctgccggcggtggtggtggcgccg

SEQ ID NO: 480 - DNA, 36 bp, Artificial Sequence
  The polynucleotide sequence that encodes advanced Macromolecule Transduction Domain (aMTD) peptide for Improvement of Cell-Permeability
gtggcgctgctggcgccggcggtggtggtggcgccg

SEQ ID NO: 481 - DNA, 57 bp, Artificial Sequence
  cDNA Sequence of Histidine Tag
atgggcagcagccatcatcatcatcatcacagcagcggcctggtgccgcgcggcagc

SEQ ID NO: 482 - protein, 19 aa, Artificial Sequence
  Amino Acid Sequence of Histidine Tag
MGSSHHHHHHSSGLVPRGS

SEQ ID NO: 483 - DNA, 36 bp, Artificial Sequence; identical to SEQ ID NO: 255
  cDNA Sequences of aMTDs and Peptides (aMTD43)

SEQ ID NO: 484 - DNA, 36 bp, Artificial Sequence; identical to SEQ ID NO: 283
  cDNA Sequences of aMTDs and Peptides (aMTD165)

SEQ ID NO: 485 - protein, 12 aa, Artificial Sequence
  Amino Acid Sequences of aMTDs and Peptides (aMTD43)
LLAAPLVVAAVP

SEQ ID NO: 486 - protein, 12 aa, Artificial Sequence
  Amino Acid Sequences of aMTDs and Peptides (aMTD165)
ALAVPVALAIVP

SEQ ID NO: 487 - DNA, 252 bp, Artificial Sequence
  cDNA Sequence of human TFF1
atggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattt

SEQ ID NO: 488 - protein, 84 aa, Artificial Sequence
  Amino Acid Sequence of human TFF1
MATMENKVICALVLVSMLALGTLAEAQTETCTVAPRERQNCGFPGVTPSQCANKGCCFDDTVRGVPWCFYPNTIDVPPEEECEF

SEQ ID NO: 489 - DNA, 552 bp, Artificial Sequence
  cDNA Sequences of SDA
atggcaaatattaccgttttctataacgaagacttccagggtaagcaggtcgatctgccgcctggcaactatacccgcgcccagttggcggcgctgggcatcgagaataataccatcagctcggtgaaggtgccgcctggcgtgaaggctatcctgtaccagaacgatggtttcgccggcgaccagatcgaagtggtggccaatgccgaggagttgggcccgctgaataataacgtctccagcatccgcgtcatctccgtgcccgtgcagccgcgcatggcaaatattaccgttttctataacgaagacttccagggtaagcaggtcgatctgccgcctggcaactatacccgcgcccagttggcggcgctgggcatcgagaataataccatcagctcggtgaaggtgccgcctggcgtgaaggctatcctctaccagaacgatggtttcgccggcgaccagatcgaagtggtggccaatgccgaggagctgggtccgctgaataataacgtctccagcatccgcgtcatctccgtgccggtgcagccgagg

SEQ ID NO: 490 - protein, 184 aa, Artificial Sequence
  Amino Acid Sequences of SDA
MANITVFYNEDFQGKQVDLPPGNYTRAQLAALGIENNTISSVKVPPGVKAILYQNDGFAGDQIEVVANAEELGPLNNNVSSIRVISVPVQPRMANITVFYNEDFQGKQVDLPPGNYTRAQLAALGIENNTISSVKVPPGVKAILYQNDGFAGDQIEVVANAEELGPLNNNVSSIRVISVPVQPR

SEQ ID NO: 491 - DNA, 297 bp, Artificial Sequence
  cDNA Sequences of SDB
atggcagaacaaagcgacaaggatgtgaagtactacactctggaggagattcagaagcacaaagacagcaagagcacctgggtgatcctacatcataaggtgtacgatctgaccaagtttctcgaagagcatcctggtggggaagaagtcctgggcgagcaagctgggggtgatgctactgagaactttgaggacgtcgggcactctacggatgcacgagaactgtccaaaacatacatcatcggggagctccatccagatgacagatcaaagatagccaagccttcggaaaccctt

SEQ ID NO: 492 - protein, 99 aa, Artificial Sequence
  Amino Acid Sequences of SDB
MAEQSDKDVKYYTLEEIQKHKDSKSTWVILHHKVYDLTKFLEEHPGGEEVLGEQAGGDATENFEDVGHSTDARELSKTYIIGELHPDDRSKIAKPSETL

SEQ ID NO: 493 - DNA, 327 bp, Artificial Sequence
  cDNA Sequences of SDC
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcc

SEQ ID NO: 494 - protein, 109 aa, Artificial Sequence
  Amino Acid Sequences of SDC
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 495 - DNA, 624 bp, Artificial Sequence
  cDNA Sequences of SDD
atgaaaaagatttggctggcgctggctggtttagttttagcgtttagcgcatcggcggcgcagtatgaagatggtaaacagtacactaccctggaaaaaccggtagctggcgcgccgcaagtgctggagtttttctctttcttctgcccgcactgctatcagtttgaagaagttctgcatatttctgataatgtgaagaaaaaactgccggaaggcgtgaagatgactaaataccacgtcaacttcatgggtggtgacctgggcaaagatctgactcaggcatgggctgtggcgatggcgctgggcgtggaagacaaagtgactgttccgctgtttgaaggcgtacagaaaacccagaccattcgttctgcttctgatatccgcgatgtatttatcaacgcaggtattaaaggtgaagagtacgacgcggcgtggaacagcttcgtggtgaaatctctggtcgctcagcaggaaaaagctgcagctgacgtgcaattgcgtggcgttccggcgatgtttgttaacggtaaatatcagctgaatccgcagggtatggataccagcaatatggatgtttttgttcagcagtatgctgatacagtgaaatatctgtccgagaaaaaa

SEQ ID NO: 496 - protein, 208 aa, Artificial Sequence
  Amino Acid Sequences of SDD
MKKIWLALAGLVLAFSASAAQYEDGKQYTTLEKPVAGAPQVLEFFSFFCPHCYQFEEVLHISDNVKKKLPEGVKMTKYHVNFMGGDLGKDLTQAWAVAMALGVEDKVTVPLFEGVQKTQTIRSASDIRDVFINAGIKGEEYDAAWNSFVVKSLVAQQEKAAADVQLRGVPAMFVNGKYQLNPQGMDTSNMDVFVQQYADTVKYLSEKK

SEQ ID NO: 497 - DNA, 300 bp, Artificial Sequence
  cDNA Sequences of SDE
gggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcaccgcgaacagattggaggt

SEQ ID NO: 498 - protein, 100 aa, Artificial Sequence
  Amino Acid Sequences of SDE
GSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 499 - DNA, 891 bp, Artificial Sequence
  cDNA Sequences of SDF
ggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggt

SEQ ID NO: 500 - protein, 297 aa, Artificial Sequence
  Amino Acid Sequences of SDF
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG